SEQUENCE LISTING (71 sequences)

SEQ ID NO: 1 - protein, 9 aa, Artificial
  Synthetic sequence
FARKGALRQ

SEQ ID NO: 2 - protein, 13 aa, Artificial
  Synthetic sequence
RFARKGALRQKNV

SEQ ID NO: 3 - protein, 18 aa, Artificial
  Synthetic sequence
RFARKGALRQKNVHEVKN

SEQ ID NO: 4 - protein, 22 aa, Artificial
  Synthetic sequence
RFARKGALRQKNVHEVKNLKGA

SEQ ID NO: 5 - protein, 13 aa, Artificial
  Synthetic sequence
RFARKGALRQLAV

SEQ ID NO: 6 - protein, 13 aa, Artificial
  Synthetic sequence
RFARKGALAQKNV

SEQ ID NO: 7 - protein, 9 aa, Artificial
  Synthetic sequence
RFARKGALR

SEQ ID NO: 8 - protein, 13 aa, Artificial
  Synthetic sequence
YYXKRKMAFFEFF

SEQ ID NO: 9 - protein, 13 aa, Artificial
  Synthetic sequence
FKLKRKGAFKKFA

SEQ ID NO: 10 - protein, 13 aa, Artificial
  Synthetic sequence
ARRKRKGAFFYGG

SEQ ID NO: 11 - protein, 13 aa, Artificial
  Synthetic sequence
RRRRRKGAFRRKA

SEQ ID NO: 12 - protein, 14 aa, Artificial
  Synthetic sequence
RFARKGALRQKNVY

SEQ ID NO: 13 - protein, 12 aa, Artificial
  Synthetic sequence
DARKGALRQNKV

SEQ ID NO: 14 - protein, 16 aa, Artificial
  Synthetic sequence
ERMRPRKRQGAVRRRV

SEQ ID NO: 15 - protein, 17 aa, Artificial
  Synthetic sequence
GPRPLFCRKGALRQKVV

SEQ ID NO: 16 - protein, 11 aa, Artificial
  Synthetic sequence
QKRPAQRSKYL

SEQ ID NO: 17 - protein, 11 aa, Artificial
  Synthetic sequence
QKRPSQRAKYL

SEQ ID NO: 18 - protein, 12 aa, Artificial
  Synthetic sequence
GGPLRRTLAVRR

SEQ ID NO: 19 - protein, 12 aa, Artificial
  Synthetic sequence
GGPLSRRLAVRR

SEQ ID NO: 20 - protein, 12 aa, Artificial
  Synthetic sequence
GGPLSRTLAVRR

SEQ ID NO: 21 - protein, 12 aa, Artificial
  Synthetic sequence
GGPLSRRLAVAR

SEQ ID NO: 22 - protein, 12 aa, Artificial
  Synthetic sequence
GGPLRRTLAVAR

SEQ ID NO: 23 - protein, 8 aa, Artificial
  Synthetic sequence
VRKALRRL

SEQ ID NO: 24 - protein, 12 aa, Artificial
  Synthetic sequence
GGRLSRTLAVAR

SEQ ID NO: 25 - protein, 17 aa, Artificial
  Synthetic sequence
TRKRQPAMRRRVHQING

SEQ ID NO: 26 - protein, 12 aa, Artificial
  Synthetic sequence
RKRQRAMRRRVH

SEQ ID NO: 27 - protein, 16 aa, Artificial
  Synthetic sequence
ERMRPRKRQGAVRRRV

SEQ ID NO: 28 - protein, 13 aa, Artificial
  Synthetic sequence
FKLKRKGAFKKFA

SEQ ID NO: 29 - protein, 13 aa, Artificial
  Synthetic sequence
YYXKRKMAFFEFF

SEQ ID NO: 30 - protein, 13 aa, Artificial
  Synthetic sequence
ARRKRKGAFFYGG

SEQ ID NO: 31 - protein, 13 aa, Artificial
  Synthetic sequence
RRRRRKGAFRRKA

SEQ ID NO: 32 - protein, 20 aa, Artificial
  Synthetic sequence
AAAKIQAAWRGHMARKKIKS

SEQ ID NO: 33 - protein, 19 aa, Artificial
  Synthetic sequence
AAAKIQAAFRGHMARKKIK

SEQ ID NO: 34 - protein, 16 aa, Artificial
  Synthetic sequence
ERMRPRKRQGAVRRRV

SEQ ID NO: 35 - protein, 8 aa, Artificial
  Synthetic sequence
VRKALRRL

SEQ ID NO: 36 - protein, 22 aa, Artificial
  Synthetic sequence
KKKKKRFSFKKAFKLSGFSFKK

SEQ ID NO: 37 - protein, 17 aa, Artificial
  Synthetic sequence
GPRPLFCRKGALRQKVV

SEQ ID NO: 38 - protein, 17 aa, Artificial
  Synthetic sequence
ESTVRFARKGALRQKNV

SEQ ID NO: 39 - protein, 16 aa, Artificial
  Synthetic sequence
ERMRPRKRQGAVRRRV

SEQ ID NO: 40 - protein, 13 aa, Artificial
  Synthetic sequence
RFARLGALRQKNV

SEQ ID NO: 41 - protein, 13 aa, Artificial
  Synthetic sequence
YYXKRKMAFFEFF

SEQ ID NO: 42 - protein, 13 aa, Artificial
  Synthetic sequence
RRFKRQGAFFYFF

SEQ ID NO: 43 - protein, 13 aa, Artificial
  Synthetic sequence
FKLKRKGAFKKFA

SEQ ID NO: 44 - protein, 13 aa, Artificial
  Synthetic sequence
ARRKRKGSFFYGG

SEQ ID NO: 45 - protein, 13 aa, Artificial
  Synthetic sequence
FKLKRKGSFKKFA

SEQ ID NO: 46 - protein, 13 aa, Artificial
  Synthetic sequence
RRFKRQGSFFYFF

SEQ ID NO: 47 - protein, 13 aa, Artificial
  Synthetic sequence
YYXKRKMSFFEFF

SEQ ID NO: 48 - protein, 13 aa, Artificial
  Synthetic sequence
RRRRRKGSFRRKA

SEQ ID NO: 49 - protein, 16 aa, Artificial
  Synthetic sequence
ERMRPRKRQGSVRRRV

SEQ ID NO: 50 - protein, 12 aa, Artificial
  Synthetic sequence
MNRRGSIKQAKI

SEQ ID NO: 51 - protein, 22 aa, Artificial
  Synthetic sequence
MFAVRDRRQTVKKGVIKAVDAV

SEQ ID NO: 52 - protein, 14 aa, Artificial
  Synthetic sequence
FGESRASTFCGTPD

SEQ ID NO: 53 - protein, 10 aa, Artificial
  Synthetic sequence
KARLSYSDKN

SEQ ID NO: 54 - protein, 13 aa, Artificial
  Synthetic sequence
SAFAGFSFVNPKF

SEQ ID NO: 55 - protein, 22 aa, Artificial
  Synthetic sequence
KKKKKRFSFKKSFKLSGFSFKK

SEQ ID NO: 56 - DNA, 21 bp, Artificial
  Synthetic sequence
ggaccacaaauucaucgcgtt

SEQ ID NO: 57 - DNA, 21 bp, Artificial
  Synthetic sequence
cgcgaugaauuuguggucctt

SEQ ID NO: 58 - DNA, 21 bp, Artificial
  Synthetic sequence
auucaucgcgcgcuucuuctt

SEQ ID NO: 59 - DNA, 21 bp, Artificial
  Synthetic sequence
gaagaagcgcgcgaugaautt

SEQ ID NO: 60 - DNA, 21 bp, Artificial
  Synthetic sequence
acaaggcuuccagugccaatt

SEQ ID NO: 61 - DNA, 21 bp, Artificial
  Synthetic sequence
uuggcacuggaagccuugutt

SEQ ID NO: 62 - DNA, 21 bp, Artificial
  Synthetic sequence
ucccuauggauccaaacggtt

SEQ ID NO: 63 - DNA, 21 bp, Artificial
  Synthetic sequence
ccguuuggauccauagggatt

SEQ ID NO: 64 - DNA, 21 bp, Artificial
  Synthetic sequence
acuuauuccugaucccaagtt

SEQ ID NO: 65 - DNA, 21 bp, Artificial
  Synthetic sequence
cuugggaucaggaauaagutt

SEQ ID NO: 66 - DNA, 21 bp, Artificial
  Synthetic sequence
auccgcaguggaaugaguctt

SEQ ID NO: 67 - DNA, 21 bp, Artificial
  Synthetic sequence
gacucauuccacugcggautt

SEQ ID NO: 68 - DNA, 21 bp, Artificial
  Synthetic sequence
agaccgacgacugucuguatt

SEQ ID NO: 69 - DNA, 21 bp, Artificial
  Synthetic sequence
uacagacagucgucggucutt

SEQ ID NO: 70 - DNA, 21 bp, Artificial
  Synthetic sequence
ggaaccacaagcaguauuctt

SEQ ID NO: 71 - DNA, 21 bp, Artificial
  Synthetic sequence
gaauacugcuugugguucctt

(19) United States
(12) Patent Application Publication
(10) Pub. No.: US 2006/0258562 A1
(43) Pub. Date: Nov. 16, 2006

(54) Methods and pharmaceutical compositions for healing wounds

(75) Inventor: Tamar Tennenbaum, Jerusalem (IL)

Correspondence Address: BROWDY AND NEIMARK, P.L.L.C.;624 NINTH STREET, NW, SUITE 300, WASHINGTON, DC, 20001-5303, US

(73) Assignee: HealOr Ltd., Ness Ziona (IL)

(21) Appl. No.: 11/332,774

(22) Filed: Jan. 17, 2006

Related U.S. Application Data
(63) Continuation-in-part of application No. PCT/IL04/00640, filed on Jul. 15, 2004.
(63) Continuation-in-part of application No. 10/644,775, filed on Aug. 21, 2003.
Application No. 10/644,775 is a continuation-in-part of application No. 10/169,801, filed on Jul. 9, 2002.
Application No. 10/169,801 is a continuation-in-part of application No. 09/629,970, filed on Jul. 31, 2000, now abandoned.
(60) Provisional application No. 60/486,906, filed on Jul. 15, 2003.

(51) Int. Cl. A61K 38/10; A61K 38/28

(52) U.S. Cl. 514/3; 514/15; 514/14; 514/13

(57) ABSTRACT

Methods and pharmaceutical compositions for inducing or accelerating a healing process of a damaged skin or skin wound, comprise modulating expression and/or activity of at least two PKC isoforms in skin cells colonizing the damaged skin or skin wound area.

### CROSS-REFERENCE TO RELATED APPLICATIONS

The present application is a continuation-in-part application of PCT application No. PCT/IL2004/000640, filed Jul. 15, 2004, in which the US is designated, and claims the benefit of U.S. Provisional Patent Application No. 60/486,906, filed Jul. 15, 2003, and U.S. patent application Ser. No. 10/644,775, filed Aug. 21, 2003, which is a continuation-in-part application of U.S. patent application Ser. No. 10/169,801, filed Jul. 23, 2002, which claims the benefit of U.S. patent application Ser. No. 09/629,970, filed Jul. 31, 2000, now abandoned, the entire contents of each and all these applications being hereby incorporated by reference herein in their entirety as if fully disclosed herein.

### FIELD AND BACKGROUND OF THE INVENTION

The present invention relates to a method and a pharmaceutical composition for inducing and/or accelerating cell proliferation and/or cell migration and/or cell differentiation and thereby accelerating the healing process of wounds. More particularly, the present invention relates to the use of modulated expression and/or activation of serine/threonine protein kinases, also known as PKCs. PKC activation is initiated by translocation to either membrane, nucleus or cytoskeletal compartments. PKC inhibition is achieved through conformation compromise by binding of specific molecules to the pseudosubstrate region and/or the ATP binding site. Thus, specific stimuli can lead to differential responses via isoform specific PKC signaling regulated by their expression, localization and phosphorylation status in particular biological settings.

The primary goal in the treatment of wounds is to achieve wound closure. Open cutaneous wounds represent one major category of wounds and include surgical wounds, burn wounds, neuropathic ulcers, pressure ulcers, venous stasis ulcers and diabetic ulcers.

Open cutaneous wounds routinely heal by a process that comprises six major components: (i) inflammation; (ii) fibroblast proliferation; (iii) blood vessel proliferation; (iv) connective tissue synthesis; (v) epithelialization; and (vi) wound contraction. Wound healing is impaired when these components, either individually or as a whole, do not function properly. Numerous factors can affect wound healing, including malnutrition, infection, pharmacological agents (e.g., actinomycin and steroids), advanced age and diabetes [see Hunt and Goodson in Current Surgical Diagnosis & Treatment (Way; Appleton & Lange), pp. 86-98 (1988)].

With respect to diabetes, diabetes mellitus is characterized by impaired insulin signaling, elevated plasma glucose and a predisposition to develop chronic complications involving several distinctive tissues. Among all the chronic complications of diabetes mellitus, impaired wound healing leading to foot ulceration is among the least well studied. Yet skin ulceration in diabetic patients takes a staggering personal and financial cost (Knighton and Fiegel, 1993; Shaw and Boulton, 1997). Moreover, foot ulcers and the subsequent amputation of a lower extremity are the most common causes of hospitalization among diabetic patients (Shaw and Boulton, 1997; Coghlan et al., 1994; Grunfeld, 1992; Reiber et al., 1998). In diabetes, the physiological process of wound healing is impaired. The defect in tissue repair has been related to several factors including neuropathy, vascular disease and infection. However, other mechanisms whereby the diabetic state associated with abnormal insulin signaling impairs wound healing and alters the physiology of skin has not been elucidated.

Another issue associated with impaired wound healing relates to infections of post surgical wounds occurring in 25% of patients hospitalized in surgical wards.

Skin is a stratified squamous epithelium in which cells undergoing growth and differentiation are strictly compartmentalized. In the physiologic state, proliferation is confined to the basal cells that adhere to the basement membrane. Differentiation is a spatial process where basal cells lose their adhesion to the basement membrane, cease DNA synthesis and undergo a series of morphological and biochemical changes. The ultimate maturation step is the production of the cornified layer forming the protective barrier of the skin (Hennings et al., 1980; Yuspa et al., 1989). The earliest changes observed when basal cells commit to differentiate is associated with the ability of the basal cells to detach and migrate away from the basement membrane (Fuchs, 1990). Similar changes are associated with the wound healing process where cells both migrate into the wound area and proliferative capacity is enhanced. These processes are mandatory for the restructuring of the skin layers and induction of proper differentiation of the epidermal layers.

The analysis of mechanisms regulating growth and differentiation of epidermal cells has been greatly facilitated by the development of culture systems for mouse and human keratinocytes (Yuspa et al., 1989; Yuspa, 1994). In vitro, keratinocytes can be maintained as basal proliferating cells with a high growth rate. Furthermore, differentiation can be induced in vitro following the maturation pattern in the epidermis in vivo. The early events include loss of hemidesmosome components (Fuchs, 1990; Hennings and Holbrook, 1983) and a selective loss of the α6β4 integrin and cell attachment to matrix proteins. This suggests that changes in integrin expression are early events in keratinocyte differentiation. The early loss of hemidesmosomal contact leads to suprabasal migration of keratinocytes and is linked to induction of Keratin 1 (K1) in cultured keratinocytes and in skin (Hennings et al., 1980; Fuchs, 1990; Tennenbaum et al., 1996a). Further differentiation to the granular layer phenotype is associated with down regulation of both β1 and β4 integrin expression, loss of adhesion potential to all matrix proteins and is followed by cornified envelope formation and cell death. Differentiating cells ultimately sloughs from the culture dish as mature squames (Yuspa et al., 1989; Tennenbaum et al., 1996b). This program of differentiation in vitro closely follows the maturation pattern of epidermis in vivo.

Recent studies in keratinocytes biology highlights the contribution of Protein Kinase C pathways, which regulate skin proliferation, migration and differentiation. The protein kinase C (PKC) family of serine-threonine kinases plays an important regulatory role in a variety of biological phenomena (Nishizuka, 1988; Nishizuka, 1989). All family members share a structural backbone, which can be divided into two major domains: a regulatory domain at the N-terminus, and a catalytic domain at the C-terminus, which contains conserved regions (C1-C4) and regions that vary between isoforms (V1-V5). In addition, PKCs exhibit a pseudosubstrate domain in the regulatory region, closely resembling the substrate recognition motif, which blocks the recognition site and prevents activation. The PKC family is composed of at least 12 individual isoforms which belong to 3 distinct categories: (i) conventional isoforms (α, β1, β2, γ) activated by Ca²⁺, phorbol esters and diacylglycerol (DAG) liberated intracellularly by phospholipase C; (ii) novel isoforms (δ, ε, η, θ) which are also activated by phorbol esters and diacylglycerol but not by Ca²⁺; and (iii) atypical (ζ, λ, ι) members of the family, which require phosphatidylserine, but are independent of Ca²⁺ and do not require DAG or phorbol esters for activation.

An important feature of PKC activation involves the association of PKC isoforms with phospholipids to form a stable membrane complex. An additional mechanism for specific translocation of PKCs from the cytosol to particulate compartments is associated with receptors for activated kinases (RACKs); this by directing their activation state and defining the subcellular distribution of these isoforms. Following activation, PKC translocation participates in determining of the functional outcome such as activation of transcription.

PKC isoforms are expressed in a variety of tissues. While some PKC isoforms (PKCα, δ, and ζ) are widely expressed in all tissues, several isoforms are expressed in a tissue-specific manner. Five PKC isoforms—α, δ, ε, η and ζ—have been identified in the epidermal layer of the skin in vivo and in culture. In addition, other skin compartments such as dermal fibroblasts, cells of the immune system and adipocytes express PKC β, θ, λ and ι Recent studies have shown that the PKC signal transduction pathway is a major intracellular mediator of the differentiation response (Denning et al., 1995; Dlugosz et al., 1990). Furthermore, pharmacological activators of PKC are powerful inducers of keratinocyte differentiation in vivo and in vitro (Yuspa, 1994; Dlugosz and Yuspa, 1993), and PKC inhibitors prevent expression of differentiation markers (Denning et al., 1995).

While conceiving the present invention, it was hypothesized that PKC isoforms modulation by over-expression and/or activation and/or inhibition may be beneficial for accelerating wound healing processes. The limitations for investigating the role of distinct PKC isoforms in skin cells proliferation and/or migration and/or differentiation has been hampered as result of the difficulty in introducing foreign genes efficiently into primary cells, by conventional methods. The short life span, differentiation potential and the inability to isolate stable transformants do not allow efficient transduction of foreign genes into primary skin cells.

Prior art describes the potential use of insulin as a therapeutic agent for healing wounds. Thus, U.S. Pat. Nos. 5,591,709, 5,461,030 and 5,145,679 describe the topical application of insulin to a wound to promote wound healing. However, these patents describe the use of insulin in combination with glucose since the function of the insulin is to enhance glucose uptake and to thus promote wound healing.

U.S. patent application Ser. No. 09/748,466 and International Patent Application No. PCT/US98/21794 describe compositions containing insulin for topical application to skin for the purpose of improving skin health or treating shallow skin injuries. However, none of these patent applications teaches the use of insulin for treating chronic, Grade II or deep wounds.

International Patent Application No. PCT/US01/10245 describes the use of cyanoacrylate polymer sealant in combination with insulin or silver for wound healing. However, the use of insulin in combination with another biologically active agent capable of modulating the expression and/or activation of PKC is not taught nor suggested in this application.

International Patent Application No. PCT/US85/00695 describes topical application of insulin for treating diabetes. However, this patent application fails to teach the use of insulin for the purpose of treating diabetes non-related wounds.

International Patent Application No. PCT/US92/03086 describes therapeutic microemulsion formulations which may contain insulin. However the use of the formulated insulin for the purpose of wound healing is not taught in this disclosure.

U.S. Pat. Nos. 4,673,649 and 4,940,660 describe compositions for clonal growth of human keratinocytes and epidermal cells in vitro which include epidermal growth factor and insulin. Both of these patents teach the use of insulin for the development of cultured skin cells which may be used for grafting. However, the application of insulin on wounds in vivo is not taught by these patents.

None of the above cited prior art references teach or suggest the use of insulin alone or in combination with another agent for modulating the expression and/or activation of PKC, so as to accelerate the healing process of wounds.

There is a widely recognized need for, and it would be highly advantageous to have, new approaches for accelerating the processes associated with wound healing. In addition, there is a widely recognized need for, and it would be highly advantageous to have, an efficient method to insert recombinant genes into skin cells which will accelerate cell proliferation and/or differentiation processes and wound healing.

### SUMMARY OF THE INVENTION

While reducing the present invention to practice the present inventors uncovered that administering insulin alone to wounds may cause adverse side effects such as excessive angiogenesis, inflammation, epidermal cells hyperplasia and scarring (see Example 23 in the Examples section hereinbelow). The present inventors further uncovered that insulin-induced side effects can be effectively circumvented while substantially accelerating the wound healing process by combining insulin with one or more agents capable of modulating expression and/or activity of PKC in skin cells colonizing the wound area.

It has further been found by the present inventors that damaged skin and skin wounds can be treated efficiently by modulating the expression and/or activity of at least two PKC isoforms in the skin damaged or wound area.

The present invention provides, in one aspect, a method of inducing or accelerating a healing process of a damaged skin or skin wound, the method comprising modulating expression and/or activity of at least two PKC isoforms in skin cells colonizing the damaged skin or skin wound area, to thereby induce or accelerate the healing process of the damaged skin or skin wound.

Hence, according to one embodiment of the present invention there is provided a method of inducing or accelerating a healing process of a damaged skin or skin wound, the method comprising the step of administering to the damaged skin or skin wound area a therapeutically effective amount of at least two PKC isoform modulating agents, each agent capable of modulating the expression and/or activity of each of said at least two PKC isoforms.

According to another aspect of the present invention there is provided a pharmaceutical composition for topical application for inducing or accelerating a healing process of a damaged skin or skin wound, the pharmaceutical composition comprising a pharmaceutically acceptable carrier and, as an active ingredient, a therapeutically effective amount of at least two PKC isoform modulating agents, each of them capable of modulating the expression and/or activity of one PKC isoform in the damaged skin or skin wound area.

According to preferred embodiments the protein kinase C isoform is selected from the group consisting of PKC-α, PKC-β(including both PKC-β1 and PKC-β2) PKC-γ, PKC-δ, PKC-ε, PKC-η, PKC-ζ, PKC-θ, PKC-λ and PKC-ι.

According to still more preferred embodiments the protein kinase C isoform is selected from the group consisting of PKC-α, PKC-β, PKC-δ, PKC-ε, PKC-η and PKC-ζ.

According to another aspect of the present invention there is provided a method of inducing or accelerating a healing process of a damaged skin or skin wound, the method comprising the step of administering to the damaged skin or skin wound area a therapeutically effective amount of copolymer-1.

According to yet another aspect of the present invention there is provided a pharmaceutical composition for inducing or accelerating a healing process of a damaged skin or skin wound, the pharmaceutical composition comprising, as an active ingredient, a therapeutically effective amount of copolymer-1 and a pharmaceutically acceptable carrier being designed for topical application of the pharmaceutical composition.

According to features in preferred embodiments of the invention described below, the wound is selected from the group consisting of an ulcer, a diabetes related wound, a burn, a sun burn, an aging skin wound, a corneal ulceration wound, an inflammatory gastrointestinal tract disease wound, a bowel inflammatory disease wound, a Crohn's disease wound, an ulcerative colitis, a hemorrhoid, an epidermolysis bulosa wound, a skin blistering wound, a psoriasis wound, seborrheic dermatitis wound, an animal skin wound, a proud flesh wound, an animal diabetic wound, a retinopathy wound, an oral wound (mucositis), a vaginal mucositis wound, a gum disease wound, a laceration, a surgical incision wound and a post surgical adhesions wound.

According to still further features in the described preferred embodiments the ulcer is a diabetic ulcer, a pressure ulcer, a venous ulcer, a gastric ulcer and an HIV related ulcer.

According to still further features in the described preferred embodiments the pharmaceutical composition is selected from the group consisting of an aqueous solution, a gel, a cream, a paste, a lotion, a spray, a suspension, a powder, a dispersion, a salve and an ointment.

According to still further features in the described preferred embodiments the pharmaceutical composition includes a solid support.

The present invention successfully addresses the shortcomings of the presently known configurations by providing new therapeutics to treat damaged skin or skin wounds.

### BRIEF DESCRIPTION OF THE FIGURES

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 demonstrates effective over-expression of PKC isoforms utilizing recombinant adenovirus vectors: Left panel: 4-day old primary keratinocytes were infected for 1 hour utilizing β-gal adenovirus (Ad-β Gal) 48 hours following infection, cells were fixed and activation of β-galactosidase protein was quantified by the induction of blue color reaction in comparison to uninfected keratinocytes. Right panel: 4-day old primary keratinocytes were infected for 1 hour utilizing recombinant isoform (PKC-α, PKC-δ, PKC-η, PKC-ζ) specific PKC adenoviruses. 24 hours later, proteins of infected (Ad) and non infected control (C) cultures were extracted for Western blot analysis and samples were analyzed using isoform specific anti-PKC antibodies as described in the Examples section below.

FIG. 2 shows that PKC activation by bryostatin 1 induces translocation of over-expressed PKC isoforms. 4-day old primary keratinocytes were infected for 1 hour with isoform (PKC-α, PKC-δ, PKC-η, PKC-ζ) specific recombinant PKC adenoviruses. 24 hours following infection, cells were either untreated (C) or stimulated with bryostatin 1 (B) for 30 minutes, and fractionated. Protein samples were subjected to Western blotting and analyzed using isoform specific anti-PKC antibodies.

FIG. 3 shows that over-expressed PKC isoforms are active in their native form. 4-day old primary keratinocytes were infected for 1 hour with isoform (PKC-α, PKC-δ, PKC-η, PKC-ζ) specific recombinant PKC adenoviruses. 18 hours following infection, cell lysates from uninfected control cells (C) and PKC isoforms over-expressing cells (OE) were immunoprecipitated using isoform specific anti-PKC antibodies. Immunoprecipitates were subjected to PKC activity assay as described in the Examples section that follows.

FIG. 4 demonstrates that over-expression of specific PKC isoforms induces distinct morphologic changes in primary keratinocytes. Primary keratinocytes were either left untreated (C) or infected with recombinant PKC α, δ, η or ζ adenoviruses. 24 hours later, cultures were observed by bright field microscopy and photographed (×20).

FIG. 5 shows distinct localization of over-expressed PKC isoforms in infected primary keratinocytes. Primary keratinocytes were plated on laminin 5-coated glass slides. Cultures were either untreated or infected with different recombinant (PKC-α, PKC-δ, PKC-η, PKC-ζ) adenoviruses. 24 hours following infection, cells were fixed, washed and air-dried. Cultures were analyzed by immunofluorescence using isoform specific anti-PKC antibodies, followed by FITC conjugated secondary antibodies. Cells were scanned by confocal microscopy and representative fields were photographed.

FIG. 6 demonstrates that PKC isoforms specifically regulate α6β4 integrin expression. 5-day old primary mouse skin keratinocytes were untreated or infected with PKCα, PKCδ, PKCη or PKCζ recombinant adenoviruses. 48 hours post infection, membranal cell fractions were subjected to SDS-PAGE electrophoresis, transferred to nitrocellulose filters, immunoblotted with anti α6 and anti-β4 antibodies and analyzed by ECL.

FIG. 7 shows that over-expression of PKCη and PKCδ induces keratinocyte proliferation. 5-day old primary mouse skin keratinocytes were untreated or infected with PKCδ, PKCα, PKCη or PKCζ recombinant adenoviruses. 48 hours post infection cell proliferation was analyzed by ³H-thymidine incorporation for 1 hour as described in experimental procedures. Results are presented as cpm/dish, in comparison to the β-galactosidase infected keratinocytes. Values are presented as mean±standard deviation of triplicate determinations in 3 separate experiments.

FIG. 8 demonstrates the PKC isoforms over-expression effects on hemidesmosomal localization of the α6β4 integrin. Primary keratinocytes were plated on laminin 5 coated glass slides and keratinocyte cultures were maintained in low Ca²⁺ MEM for 48 hours. Following that period of time, cultures were left untreated (A), or infected with PKCα, PKCδ, PKCη or PKCζ recombinant adenoviruses (B-E, respectively). 24 hours post infection, keratinocytes were fixed with 4% paraformaldehyde followed by mild extraction with 0.2% Triton-X-100, washed in PBS and air dried as described in the experimental procedures. Cultures were subjected to immunofluorescence analysis utilizing isoform specific anti-α6 antibodies, followed by FITC conjugated secondary antibodies, as described in experimental procedures.

FIGS. 9A-9B show that over-expressed PKCδ and PKCζ induce keratinocyte detachment in vitro. (A) Primary keratinocytes were either untreated (C) or infected with recombinant PKC α, δ, η or ζ adenoviruses. Cell attachment was analyzed 24 and 48 hours following infection, by lifting the cells and replating them on matrix coated dishes. Cell counts are presented as protein concentration (mg/dish) of the attached cells. (B) Primary keratinocytes were either untreated (C) or infected with recombinant PKC α, δ, η or ζ adenoviruses. Cell detachment was analyzed 24 hours following infection, by collecting the detached floating cells in the culture medium. Cell counts are presented as protein concentration (mg/dish) of the detached cells.

FIG. 10 demonstrates that PKCη is expressed in actively proliferating keratinocytes. Primary keratinocytes were plated on laminin 5-coated glass slides. 48 hours following plating keratinocytes were incubated with BrdU solution for 1 hour followed by immunofluorescence analysis using anti-PKCη (red) and anti BrdU (green) antibodies as described in the Examples section that follows. Cells were scanned by confocal microscopy and representative fields were photographed.

FIG. 11 demonstrates that PKCη induces, while PKCη mutant reduces, keratinocyte proliferation. Primary skin keratinocytes were infected for 1 hour with recombinant PKCη or a dominant negative mutant of PKCη (DNPKCη or PKC DNη) adenoviruses. 48 hours post infection, cell proliferation was analyzed by 1-hour ³H-thymidine incorporation as described in the Examples section that follows. Results are presented as cpm/dish. Control-uninfected cells.

FIGS. 12A-12B demonstrate that PKCη and DNPKCη over-expressions specifically regulate PKC localization and cellular morphology. Primary skin keratinocytes were infected for 1 hour with recombinant PKCη or a dominant negative mutant of PKCη (PKC DNη) adenoviruses. 48 hours post infection, keratinocytes were fixed and subjected to (A) bright field photography (×20) and (B) immunofluorescence analysis utilizing PKCη specific antibodies followed by FITC conjugated secondary antibodies as described in experimental procedures. Control-uninfected cells.

FIGS. 13A-13B show that inhibition of PKCη expression induces keratinocyte differentiation in proliferating keratinocytes. Primary skin keratinocytes were either maintained proliferating in low Ca²⁺ medium or differentiated in 0.12 mM Ca²⁺ for 24 hours. Thereafter, keratinocytes were infected for 1 hour with recombinant PKCη or a dominant negative mutant of PKCη (PKC DNη) adenoviruses. 24 hours after infection, keratinocytes were either maintained in low Ca²⁺ medium or transferred to differentiating medium containing 0.12 mM Ca²⁺ for an additional 24 hours. 48 hours after infection, keratinocytes were extracted and subjected to SDS-PAGE gels. PKCη (A) and keratin 1 (B) expression was analyzed by Western blotting.

FIG. 14 demonstrates that topical in vivo expression of PKCη enhances the formation of granulation tissue and accelerates wound healing in mice incisional wounds. Whole skin 7 mm incisions were created on the back of nude mice. Topical application of control β-gal, PKCη and PKCα adenovirus suspension was applied at 1d and 4d following wounding. Whole skin wounds were fixed in 4% paraformaldehyde and skin sections were analyzed histologically by H&E staining and bright field microscopy. E-epidermis, D-dermis.

FIG. 15 demonstrates that insulin, but not IGF1, specifically induces translocation of PKCδ in proliferating keratinocytes. Primary keratinocytes were isolated and plated as described in the Examples section that follows. Proliferating keratinocytes were maintained for five days in low Ca²⁺ medium (0.05 mM) until they reached 80% confluency. Cells were stimulated with 10⁻⁷ M insulin (Ins) or 10⁻⁸ M IGF1 (IGF) for 15 minutes. Cells were lysed, as described, and 20 μg of membrane or cytosol extracts of stimulated and control un-stimulated (Cont) cells were subjected to SDS-PAGE and transfer. Blots were probed with specific polyclonal antibodies to each PKC isoform.

FIG. 16 shows that insulin but not IGF1 induces PKCδ activity. To determine PKCδ activity, 5-day keratinocyte cultures were stimulated with 10⁻⁷ M insulin (Ins) or 10⁻⁸ M IGF1 (IGF) for the designated times (1, 15 or 30 minutes). PKCδ was immunoprecipitated from membrane (blue bars, mem) and cytosol (purple bars, cyto) fractions using specific anti-PKCδ antibody. PKCη immunoprecipitates were analyzed for PKC activity utilizing an in vitro kinase assay as described in experimental procedures. Each bar represents the mean±SE of 3 determinations in 3 separate experiments. Values are expressed as pmol ATP/dish/min.

FIGS. 17A-17B show that insulin and IGF1 have an additive effect on keratinocyte proliferation. Proliferating keratinocytes were maintained for five days in low Ca²⁺ medium (0.05 mM) until they reached 80% confluence. (A) 5-day keratinocyte cultures were stimulated for 24 hours with insulin or IGF1 at the designated concentrations. (B) In parallel, keratinocytes were stimulated with 10⁻⁷ M insulin (Ins) and increasing doses of IGF1 (IGF). At each concentration the right column (striped bar) represents proliferation observed when both hormones were added together. The left bar demonstrates the separate effect of 10⁻⁷ M insulin (red bars) and increasing concentrations of IGF1 (gray bars). Thymidine incorporation was measured as described in experimental procedures. The results shown are representative of 6 experiments. Each bar represents the mean±SE of 3 determinations expressed as percent above control unstimulated keratinocytes.

FIGS. 18A-18B demonstrate the over-expression of recombinant PKC adenovirus constructs. Keratinocyte cultures were infected utilizing recombinant adenovirus constructs containing wild type PKCδ (WTPKCδ), wild type PKCα (WTPKCα), or a dominant negative PKCδ mutant (DNPKCδ). (A) Following infection, cells were cultured for 24 hours, harvested, and 20 μg of protein extracts were analyzed by Western blotting using specific anti PKCα or anti PKCδ antibodies. The blots presented are representative of 5 separate experiments. (B) 24 hours following infection, cells were harvested and PKCα or PKCδ immunoprecipitates were evaluated by in vitro kinase assay.

FIG. 19 shows the effects of PKC over-expression on insulin or IGF1-induced proliferation. Non-infected (light blue bars), or cells over-expressing WTPKCδ (dark blue bars) or DNPKCδ (slashed blue bars) were treated for 24 hours with 10⁻⁷ M insulin (Ins), 10⁻⁸ M IGF1 (IGF) or both (Ins+IGF). Thymidine incorporation was measured as described in experimental procedures. Each bar represents the mean±SE of 3 determinations in 3 experiments done on separate cultures. Values are expressed as percent of control, unstimulated cells from the same culture in each experiment.

FIG. 20 shows that inhibition of PKCδ activity specifically abrogates insulin induced keratinocyte proliferation. Primary keratinocytes were cultured as described in the Examples section that follows. Non-infected cells or keratinocytes infected with DNPKCδ were stimulated for 24 hours with the following growth factor concentrations: 10⁻⁷ M insulin (Ins), 10⁻⁸ M IGF1 (IGF), 10 ng/ml EGF, 10 ng/ml PDGF, 1 ng/ml KGF or 5 ng/ml ECGF. Thymidine incorporation was measured as described in the Examples section that follows. Each bar represents the mean±SE of 3 determinations in 3 experiments done on separate cultures. Values are expressed as percent of control, un-stimulated cells from the same culture in each experiment.

FIG. 21 shows that over-expression of PKCδ mediates specifically insulin induced keratinocyte proliferation. Primary keratinocytes were cultured as described under FIG. 1. Non-infected cells or keratinocytes infected with over-expressed WTPKCδ were stimulated for 24 hours with the following growth factor concentrations: 10⁻⁷ M insulin (Ins), 10⁻⁸ M IGF1 (IGF), 10 ng/ml EGF, 10 ng/ml PDGF, 1 ng/ml KGF or 5 ng/ml ECGF. Thymidine incorporation was measured as described in the Examples section that follows. Each bar represents the mean±SE of three determinations in three experiments done on separate cultures. Values are expressed as percent of control, unstimulated cells from the same culture in each experiment.

FIGS. 22A-22B substantiate the significance of PKCδ and PKCζ in the wound healing process of skin in vivo. Utilizing in vivo mouse model of newly developed isoform specific PKC null mice, PKCα, PKCδ and PKCζ null mice and their wild type littermates were subjected to a wound healing study. Mice were anesthetized and skins through punch biopsies of 4 mm in diameter were created on the mice back. After a week follow-up, mice skin was removed and skin wound healing was quantified by subjecting skin flaps to a wound strength test utilizing a bursting chamber technique. Values are expressed as bursting pressure that represents the maximal pressure within the chamber monitored until bursting occurs. Results represent determinations obtained in distinct groups of 12-20 mice. Experiments were repeated at least 3 times.

FIG. 23 identifies a specific interaction between STAT3 and PKCδ in primary skin keratinocytes. Primary keratinocytes were either untreated (upper panel) or infected for 1 hour with isoform specific, recombinant PKC α, δ, η or ζ adenoviruses (lower panel). Cells were extracted and immunoprecipitated (IP) with isoform specific PKC antibodies. The immunoprecipitates were subjected to Western blot analysis using anti-PKCs or anti-STAT3 antibodies.

FIG. 24 demonstrates the importance of PKCδ activation to insulin induced transcriptional activation of STAT3. Primary keratinocytes were plated on glass slides and maintained for 5 days in low Ca⁺⁺ medium (0.05 mmol/l) until they reached 80% confluency. Cells were untreated (Cont, upper panel) or pre-treated with 5 μM Rottlerin for 7 minutes (R, lower panel), followed by 10⁻⁷ M insulin for 5 minutes (Ins). Cells were fixed by methanol, washed and air-dried. Cultures were analyzed by immunofluorescence using antiphospho-Tyr-705-STAT3 antibody, followed by FITC conjugated secondary antibody. Cells were scanned by confocal microscopy.

FIG. 25 demonstrates that over-expression of DN PKCδ inhibits keratinocyte proliferation induced by over-expression of PKCδ and STAT3. Primary keratinocytes were infected for 1 hour with recombinant adenovirus constructs containing β-Gal (for control), PKC δ, WT STAT3, DN STAT3 or double-infected with DN PKCδ, followed by STAT3. 24 hours following infection, cell proliferation was analyzed by 1-hour ³H-thymidine incorporation. The results are presented as DPM/mg protein. Each bar represents the mean of three determinations in a plate from the same culture.

FIG. 26 demonstrates the importance of insulin concentrations and frequency of applications on wound healing in vivo. Wound incisions were performed on the back of 8-10 week old C57BL mice and were treated with PBS (control) or with different concentrations and frequencies of insulin applications (i.e., seven daily repeat applications vs. a single application). The mice were sacrificed seven days after wounding and the areas of treated wounds were measured. The results are presented as mm² wound area and each bar represents the mean of six replications±standard deviation (p<0.005).

FIG. 27 demonstrates histological effects of insulin concentrations and frequency of applications on wound healing in vivo. Wound incisions were performed on the back of 8-10 week old C57BL mice and were treated with different concentrations of insulin and frequencies of applications (i.e., seven daily repeat applications vs. a single application). Histological wound sections were performed seven days after wounding and were analyzed for epidermal and dermal closure (wound contraction). Epidermal closure was assessed by Keratin 14 (K14) antibody staining (left panel) and was considered positive if the wound was stained positive across the entire gap. The dermal closure was considered positive if both dermal wound sides could be observed under a light microscope in a single field at ×100 magnification (right panel). The results are presented as percent of wound closure over control and each bar represents the mean of six replications.

FIG. 28 demonstrates a synergistic effect of combining insulin and platelet-derived growth factor (PDGF-BB) on wound healing in vivo. Wound incisions were performed on the back of 8-10 week old C57BL mice and were treated with a single application of insulin, PDGF-BB, or with insulin and PDGF-BB combined. The treated mice were sacrificed seven days after wounding and biopsies were taken for histological analyses of epidermal and dermal closure (wound contraction). Epidermal closure was assessed by Keratin 14 (K14) antibody staining (left panel) and was considered positive if the wound was stained positive across the entire gap. The dermal closure was considered positive if both dermal wound sides could be observed under a light microscope in a single field at ×100 magnification (right panel). The results are presented as were summarized in a bar graph as percent of wound closure over control and each bar represents the mean of six replications.

FIGS. 29A-29D are photographs illustrating the morphological effect of combining insulin and a PKCα inhibitor on wound healing in vivo. Wound incisions were performed on the back of 8-10 week old C57BL mice and were either untreated (control) or treated with insulin (HO/01) combined with a PKCα inhibitor (HO/02). Skin biopsies were removed 7 days after wounding for morphological observations. A-B show control wounds while C-D show treated wounds.

FIG. 30 is a histo-micrograph illustrating the combined effect of insulin and a PKCα inhibitor on dermal closure (wound contraction). Wound incisions were performed on the back of 8-10 week old C57BL mice and were either untreated (control) or treated daily with insulin (HO/01) combined with a PKCα inhibitor (HO/02). The treated mice were sacrificed 7 days after wounding. Histological wound sections were performed and observed under a light microscope. The dermal closure was considered positive if both dermal wound sides could be observed in a single ×100 magnification field The opened wound area in the untreated control section (left panel) was too large to be contained in a single ×100 magnification field, while the treated wound section (right panel) shows a positive dermal closure. The yellow speckled lines mark the dermal edges.

FIG. 31 is a histo-micrograph illustrating the combined effect of insulin and a PKCα inhibitor on epidermal closure. Wound incisions were performed on the back of 8-10 week old C57BL mice and were either untreated or treated daily with insulin (HO/01) combined with a PKCα inhibitor (HO/02). The treated mice were sacrificed seven days after wounding. Histological wound sections were performed, stained with keratin 14 (indicative of basal keratinocytes) and observed under a light microscope. The opened wound area (arrow marked) in the untreated control section (left panel) was too large to be contained in a single ×100 magnification field, while the treated wound section (right panel) shows an epidermal closure through the entire wound gap.

FIG. 32 is a histo-micrograph illustrating the combined effect of insulin and a PKCα inhibitor on spatial differentiation of epidermal cells. Wounded mice (C57BL, 8-10 week old) were treated daily with topical applications of insulin (HO/01) combined with a PKCα inhibitor (HO/02). The treated mice were sacrificed seven days after wounding. Histological wound sections were performed and stained with keratin 1 (K1) antibody, which highlights the initial stage of spatial cell differentiation. The untreated control section (left panel) shows a vast undifferentiated wound area (marked by the arrow), while a massive epidermal reconstruction can be observed in the treated wound section (right panel).

FIG. 33 demonstrates the quantitative effect of insulin combined with a PKCα inhibitor on wound healing in vivo. Wounded mice (C57BL, 8-10 week old) were treated daily with topical applications of insulin (HO/01) combined with a PKCα inhibitor (HO/02). The treated mice were sacrificed seven days after wounding. Histological wound sections were performed and analyzed for dermal contraction, epidermal closure and spatial differentiation as described in FIGS. 30-32. The bar graph shows the incidence (percentage) of fully healed wounds as determined by histological analyses within each treatment group.

FIGS. 34A-34G are photographs illustrating the combined effect of inhibiting expression and/or activity of PKCα and modulating expression and/or activity of another PKC isoform in dermal cells, or administering a hormone to the dermal cells, on the closure of in vitro skin wounds. Cultured primary skin fibroblasts were infected with dominant negative (DN) kinase inactive PKCα. 24 hours later scratches were performed and the cultures were either left untreated (A), or infected with wild type (WT) PKCδ (B), PKCη (C), WT PKCζ (D) or WT PKCε (E). Alternatively, PKCα-inhibited cultures were treated with adipsin (2 μg/ml; F) or insulin (6.7×10⁻⁷ M; G). Photographs were taken 24 hours following treatment.

FIGS. 35A-35H are photographs illustrating the combined effect of inhibiting expression and/or activity of PKCα and modulating expression and/or activity of another PKC isoform in dermal cells, or administration of a growth factor, an adipokine or a PKC isoform RACK peptide to the dermal cells, on the closure of in vitro skin wounds. Cultured primary skin keratinocytes were infected with dominant negative (DN) kinase inactive PKCα. 24 hours later scratches were performed and the cultures and were either left untreated (A) or infected with wild type (WT) PKCε (D), WT PKCζ (E) or WT PKCη (F). Alternatively, PKCα-inhibited cultures were treated with IL-6 (1 μg per dish; B), KGF (1 μg per dish; C), PKCδ RACK (10⁻⁷ M; H) or TNFα (12 μg/ml; G). Photographs were taken 24 hours following treatment.

FIGS. 36A-36B are photographs illustrating the combined effect of inhibiting expression and/or activity of PKCζ in dermal cells and administering a growth factor to the dermal cells, on the closure of in vitro skin wounds. Cultured primary skin fibroblasts were infected with dominant negative (DN) kinase inactive form of PKCζ (DNζ). 24 hours later scratches were performed and the cultures were either left untreated (A) or treated with KGF (1 μg per dish; B). Photographs were taken 24 hours following treatment.

FIGS. 37A-37D are photographs illustrating the combined effect of inhibiting expression and/or activity of PKCζ in dermal cells and administering an adipokine to the dermal cells, on the closure of in vitro skin wounds. Cultured primary skin keratinocytes were infected with dominant negative (DN) kinase inactive form of PKCζ (DNζ). 24 hours later scratches were performed and the cultures were either left untreated (A) or treated with IL-6 (1 μg per dish; B), TNFα (12 μg/ml; C) or adiponectin (1 μg per dish; D). Photographs were taken 24 hours following treatment.

FIGS. 38A-38E are photographs illustrating the combined effect of inhibiting expression and/or activity of PKCβ in dermal cells and administering a growth factor, an adipokine, insulin or GW9662 to the dermal cells, on the closure of in vitro skin wounds. Cultured primary skin fibroblasts were infected with dominant negative (DN) kinase inactive form of PKCβ (DNβ). 24 hours later scratches were performed and the cultures were either left untreated (A) or treated with KFG (1 μg per dish; B), IL-6 (1 μg per dish; C), insulin (6.7×10⁻⁷ M; D) or GW9662 (1 μg per dish; E). Photographs were taken 24 hours following treatment.

FIGS. 39A-39E are photographs illustrating the combined effect of promoting expression and/or activity of PKCδ and modulating expression and/or activity of another PKC isoform in dermal cells, or administering an adipokine to the dermal cells, on the closure of in vitro skin wounds. Cultured primary skin keratinocytes were infected with wild type (WT) kinase form of PKCδ (DNδ). 24 hours later scratches were performed and the cultures were either left untreated (A) or infected with WT PKCζ (PKCζ; B), WT PKCε (PKCε; C) or DN PKCα (PKCα; D). Alternatively, PKCε-promoted cultures were treated with adipsin (2 μg/ml; E). Photographs were taken 48 hours following treatment.

FIGS. 40A-40F are photographs illustrating the effect of administering Copolymer-1, insulin, the PKCα inhibitor (HO/02), or combinations thereof, on the closure of in vitro skin wounds. Cultured primary skin keratinocytes were either left untreated (A), or treated with insulin only (6.7×10⁻⁷ M; B), Copolymer-1 only (55 μg/dish; C), a mixture of insulin and PKCα inhibitor (HO/02) (6.7×10⁻⁷ M and 10⁷M, respectively; D), a mixture of Copolymer-1 and insulin (55 μg/dish and 6.7×10⁻⁷ M, respectively; E) or a mixture of Copolymer-1, insulin and PKCα inhibitor (HO/02) (55 μg/dish, 6.7×10⁻⁷M and 10⁷M, respectively; F). Photographs were taken 48 hours following treatment.

FIGS. 41A-41D are photographs illustrating the effect of Copolymer-1, insulin, PKCα inhibitor (HO/02), or combinations thereof, on wound healing in vivo. Wounded mice were either left untreated (A) or treated daily for 4 days with topical applications of Copolymer-1 (55 μg/ml: B), a mixture of Copolymer-1 and insulin (55 μg/ml and 1 μM, respectively; C), or a mixture of Copolymer-1, insulin and PKCα inhibitor (HO/02) (55 μg/ml, 1 μM and 1 μM, respectively; D). Photographs were taken 4 days post wounding.

FIGS. 42A-42H are histo-micrographs illustrating the effect of thymus proximity to the wound gap on the wound healing process. A-B—show normal adult rodent thymus at ×200 magnification. C—a 7-day old wound magnified at ×40, thymus is observed in close proximity of the wound gap (in red square; magnified at ×200 in D). The wound is re-epithelized, granulation tissue is formed and dermal contraction is in progress. E-F—show a 9-day old wound of a STZ diabetes mouse magnified at ×40 (E) and ×200 (F), no thymus is observed in close proximity of the wound gap and no re-epithelization, tissue granulation, or dermal contraction is observed. G—shows a 9-day old wound of a STZ diabetic mouse magnified at ×40. The wound was treated with a mixture (HO/03/03) of insulin and PKCα inhibitor (HO/02). Thymus is observed in close proximity of the wound gap (in red square; magnified ×20 in H). The wound is re-epithelized, granulation tissue is formed and dermal contraction is in progress.

FIG. 43 is a photograph illustrating the effect of insulin combined with the PKCα inhibitor HO/02 on the healing of wounds and damaged skin. Longitudinal wound incisions were effected on the back of Large Whites & Landrace domestic pigs and treated daily for 15 days with either PBS (control) or a mixture (HO/03/03) of 1 μM insulin and 1 μM PKCα inhibitor HO/02. The wounds were photographed 30 days post wounding. The HO/03/03 treated wounds are completely healed with no scar formation and exhibit markedly improved skin aesthetics as compared with the buffer control.

FIG. 44 shows that Copolymer-1 inhibits PKCη activity in keratinocytes in vitro. For the PKC activity assay, plated primary keratinocytes were incubated with 2 concentrations of Copolymer-1 (Cop-1): 55 μg/kg and 5 μg/kg for either 10 minutes or 5 hours. Specific PKCη activity was measured with the use of the SignaTECT Protein Kinase C Assay System (Promega, Madison, Wis., USA) according to the manufacturer's instructions. Control panels are plates that did not receive Cop-1. Results are presented as cpm per μg protein.

FIG. 45 is a graph illustrating the effect of Copolymer-1 (COP-1), insulin, PKCα inhibitor (HO/02), or combinations of COP-1 and insulin or COP-1 and HO/02 on epidermal closure of acute wounds. The graph summarizes two separate experiments (n=54). The skin sections were removed 7-days post wounding. Paraffin embedded sections were subjected to immunohistochemical keratin-14 staining. The results are summarized as percent of healed wounds from total wounds in the specific group.

FIG. 46 shows histological illustration of healing progression, 7 days post wounding, after a combined treatment with Cop-1 and insulin. C57BL/6J mouse histological section stained for H&E (Hematoxylin and Eosin). Epidermis and hair follicles are indicated by the purple stain; dermis is indicated by the pink stain. Black box indicates edges of epidermis. Red lines and arrows indicate wound edges. Microscope magnification ×100.

FIG. 47 shows the effect of the HO/03/03 formulation (mixture of 1 μM insulin and 1 μM PKCα pseudosubstrate N-myristoylated peptide of SEQ ID NO:1) on acute wound healing in dogs. 4 separate post surgical wounds of female dogs were daily treated with either PBS (control) or with formulation HO/03/03 (1 μg in PBS per 1 cm²). HO/03/03 was applied on pad and padded topically to the wound and each treatment was performed for 20 minutes. Photo-documentation represent wounds 5 days post surgery. Black bar represent 1 cm.

FIG. 48 shows the effect of the HO/03/03 formulation on chronic wound healing in a horse. Two “proud flesh” chronic wounds, on separate limbs of a horse that had a history of recurring wounds for the past 5 years, were treated daily with either PBS (control) or with formulation HO/03/03 (1 μg in PBS per 1 cm²). Wounds presented at the upper panel failed to heal for the past 4 months and exhibit common features of ‘proud flesh’ wounds: overgrowth of granulation tissue with very high density of blood vessels. HO/03/03 was applied on pad gauze and bandaged to the wound using saran wrap. Each treatment was performed for 30 minutes. Following treatment bandage was removed and wounds remained undressed. Results were photo-documented on day 30 of treatment. Black bar represent 1 cm.

FIG. 49 shows the effect of the HO/03/03 formulation on healing of dog burns. A 30 kg female dog suffering burns from unknown origin was treated with antibiotics and irrigation of wound for a period of over 40 days. However, the wound failed to close and was subjected to recurring infections, to a stage that the dog developed immunity to the antibiotics and veterinarian suggested putting the dog to sleep. Treatment with HO/03/03 was applied daily on pad and padded topically to the wound. Each treatment was performed for 20 minutes. Wound was left un-bandaged post treatments.

### DESCRIPTION OF THE PREFERRED EMBODIMENTS

The present invention relates to methods and pharmaceutical compositions designed for modulating the expression and/or activaty of at least two serine/threonine protein kinases, also known as PKCs, in skin cells colonizing the damaged skin or skin wound area, for inducing and/or accelerating cell proliferation and/or migration and/or cell differentiation, and thereby accelerate the healing process of a damaged skin or skin wound.

The principles and operation of the methods and pharmaceutical compositions according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or exemplified in the Examples section. The invention is capable of other embodiments or of being practiced or carried out in various ways. Also, it is to be understood that the phraseology and terminology employed herein is for the purpose of description and should not be regarded as limiting.

Adult skin consists of several layers including: a keratinized stratified epidermis, an underlying thick layer of collagen-rich dermal connective tissue providing support and nourishment and a subcutaneous adipose tissue. Skin serves as the protective barrier against exogenous pathogens and environmental stress. Therefore any injury or break in the skin must be rapidly and efficiently mended. As described in the Background section hereinabove, the first stage of the repair is achieved by formation of the clot that plugs the initial wound. Thereafter, inflammatory cells, fibroblasts and capillaries invade the wound bed to form the granulation tissue. The following stages involve re-epithelization of the wound where basal keratinocytes have to lose their hemidesmosomal complexes; keratinocytes migrate upon the granulation tissue to cover the wound. Following keratinocyte migration, keratinocytes enter a proliferative boost, which allows replacement of cells lost during the injury. Concomitantly to keratinocyte migration and proliferation, these cells undergo differentiation processes forming the new stratified epidermis. (Weinstein, 1998; Singer and Clark, 1999; Whitby and Ferguson, 1991; Kiritsy et al., 1993). Several growth factors have been shown to participate in this process including EGF family of growth factors, KGF, PDGF and TGFβ1 (Whitby and Ferguson, 1991; Kiritsy et al., 1993; Andresen et al., 1997). Among these growth factors both EGF and KGF are thought to be intimately involved in the regulation of proliferation and migration of epidermal keratinocytes (Werner et al., 1994; Threadgill et al., 1995). Fundamental to the understanding of wound healing biology is knowledge of the signals that trigger the cells in the wound to migrate, proliferate, and lay down new matrix in the wound gap.

To facilitate understanding of the invention set forth in the disclosure that follows, a number of terms are defined below.

The term “wound” refers broadly to injuries to the skin and subcutaneous tissue initiated in any one of a variety of ways (e.g., pressure sores from extended bed rest, wounds induced by trauma, cuts, ulcers, burns and the like) and with varying characteristics. Wounds are typically classified into one of four grades depending on the depth of the wound: (i) Grade I: wounds limited to the epithelium; (ii) Grade II: wounds extending into the dermis; (iii) Grade III: wounds extending into the subcutaneous tissue; and (iv) Grade IV (or full-thickness wounds): wounds wherein bones are exposed (e.g., a bony pressure point such as the greater trochanter or the sacrum).

The term “partial thickness wound” refers to wounds that encompass Grades I-III; examples of partial thickness wounds include burn wounds, pressure sores, venous stasis ulcers, and diabetic ulcers.

The term “deep wound” is meant to include both Grade III and Grade IV wounds.

The term “healing” in respect to a wound refers to a process to repair a wound as by scar formation.

The phrase “inducing or accelerating a healing process of a skin wound” refers to either the induction of the formation of granulation tissue of wound contraction and/or the induction of epithelialization (i.e., the generation of new cells in the epithelium). Wound healing is conveniently measured by decreasing wound area.

The present invention contemplates treating all wound types, including deep wounds and chronic wounds.

The term “chronic wound” refers to a wound that exhibits impaired healing parameters interfering with the physiological sequence of events. These wounds tend to prolong and/or halt healing time course, subjecting the wounds to further complications such as recurrent infections and necrosis.

The phrase “skin wound” as used herein refers to any type of epithelial wound including, but not limited to, an ulcer such as a diabetic ulcer, a pressure ulcer, a venous ulcer, a gastric ulcer and an HIV-related ulcer, a diabetes-related wound, a burn, a sun burn, an aging skin wound, a corneal ulceration wound, an inflammatory gastrointestinal tract disease wound, a bowel inflammatory disease wound, a Crohn's disease wound, an ulcerative colitis, a hemorrhoid, an epidermolysis bulosa wound, a skin blistering wound, a psoriasis wound, an animal skin wound, a proud flesh wound, an animal diabetic wound, a retinopathy wound, an oral wound (mucositis), a vaginal mucositis wound, a gum disease wound, a laceration, a surgical incision wound and a post surgical adhesions wound.

The phrase “skin damage” as used herein refers to any type of skin damage or condition such as, for example, wrinkles (e.g., ultraviolet irradiation-induced wrinkles), skin lines, crevices, bumps, large pores (e.g., associated with adnexal structures such as sweat gland ducts, sebaceous glands, or hair follicles), or unevenness or roughness, loss of skin elasticity (loss and/or inactivation of functional skin elastin), sagging (including puffiness in the eye area and jowls), loss of skin firmness, loss of skin tightness, loss of skin recoil from deformation, discoloration (including undereye circles), blotching, sallowness, hyperpigmented skin regions such as age spots and freckles, keratoses, abnormal differentiation, hyperkeratinization, elastosis, collagen breakdown, and other histological changes in the stratum corneum, dermis, epidermis, the skin vascular system (e.g., telangiectasia or spider vessels), and underlying tissues, especially those proximate to the skin.

The term “PKC isoform” as used herein encompasses all PKC isoforms including PKC-α, PKC-β, PKC-δ, PKC-ε, PKC-η, PKC-ζ, PKC-γ, PKC-θ, PKC-λ and PKC-ι. The term “PKC-β” is used to denote both PKCβ1 and PKCβ2, but sometimes in the invention the isoform PKCβ2 is preferred.

The phrase “modulating expression and/or activity of a PKC isoform” relates to an increased or reduced expression and/or activity of a PKC isoform. Increase of the expression leads to increased production of the PKC isoform. The term “activator” is used herein to describe a molecule that enhances expression and/or activity of a PKC isoform. The term “inhibitor” is used herein to describe a molecule that inhibits expression and/or activity of a PKC isoform. Among others, the phosphoryl transfer region, the pseudosubstrate domain, the phorbolester binding sequences, and the phosphorylation sites may be targets for modulation of isoenzyme-specific PKC activity (Hofmann, 1997). It should be understood that many modulators are not specific and sometimes the modulator may be both inhibitor and activator.

The “pseudosubstrate region” or autoinhibitory domain of a PKC isoform is defined as a consensus sequence of substrates for the kinase with no phosphorylatable residue. The pseudosubstrate domain is based in the regulatory region, closely resembling the substrate recognition motif, which blocks the recognition site and prevents phosphorylation. Thus, inhibitory peptides are obtained by replacing a phosphorylatable residue of serine (S) or tyrosine (T) by alanine (A). PKCδ is the only PKC isoform known to have additional binding site enabling the isoform's activation on the C2 domain, the conserved domain 2 of PKCδ (Benes et al., 2005, incorporated herein by reference in its entirety as if fully disclosed herein).

The term “adipokine” as used herein relates to a group of cytokines (cell-to-cell signalling proteins) secreted by adipose tissue. Examples of adipokines that can be used according to the invention include adipsin, adiponectin, apelin, visfatin, resistin, leptin, lipoprotein lipase, plasminogen activator inhibitor-1 (PAI-1), IL-4, IL-6, TNF-α, IL-1β, angiotensin I-IV (angiotensin IV is an active angiotensin II fragment) and cycloanalogues thereof, angiotensinogen, 1-butyrylglycerol, matrix metalloproteinase 2, matrix metalloproteinase 9, and vascular endothelial growth factor (VEGF).

PKC is a major signaling pathway, which mediates keratinocyte proliferation, migration and differentiation. PKC isoforms α, β, δ, ε, η and ζ are expressed in the skin (Reynolds et al., 1995; Yuspa, 1994; Denning et al., 1995). In previous application Ser. No. 09/629,970 and Ser. No. 10/169,801, the inventors hypothesized that PKC modulated expression and/or activity may induce cell proliferation and/or cell migration and/or cell differentiation and thereby accelerate the healing process of wounds. While reducing the invention of said previous applications to practice this theory has been approved by numerous experiments showing that PKC modulated expression and/or activity indeed induces cell proliferation and cell differentiation and accelerates the healing process of wounds. As further delineated in said previous applications in great detail, various distinct approaches were undertaken to modulate expression and/or activity of PKC to thereby accelerate the healing process of wounds. Based on the experimental findings, other approaches have been devised. A striking and novel phenomenon was discovered while reducing the invention in said previous applications to practice, namely, that insulin serves as a modulator of expression and/or activity of PKC. As such, insulin was described as a therapeutic agent for modulating the expression and/or activity of PKCδ so as to accelerate the healing process of wounds.

The characteristics of distinct PKC isoforms and their specific effects on cell proliferation and/or differentiation are of great importance to the biology of skin wound healing. Utilizing PKC adenovirus constructs enabled to identify the specific roles of a variety of PKC isoforms in the wound healing process in vitro and in vivo. All isoforms were able to specifically affect different aspects of keratinocyte growth and differentiation. Two isoforms, PKCδ and PKCζ, could specifically regulate integrin regulation (see Example 6 below), adherence to the basement membrane (see Example 9 below) and hemidesmosome formation (see Example 8 below). Two isoforms, PKCδ and PKCη, were found to regulate the proliferation potential of epidermal keratinocytes (see Examples 7 and 11 below). In addition, a dominant negative isoform of PKCη (DNPKCη) was able to specifically induce differentiation in actively proliferating keratinocytes (see Example 12 below). Finally, the importance of distinct PKC isoforms to the wound healing process in skin was also verified in an in vivo system. Utilizing PKC null mice where expression of distinct PKC isoforms was abolished it is shown herein that PKCδ and PKCζ, which were found to be required for both adhesion and motility processes in skin keratinocytes, are also important in the in vivo wound healing process in an animal model (see Example 19). Full thickness skin biopsies in PKC null skin suggested that both PKCδ and PKCζ, but not PKCα, are essential for proper healing of the wound. Furthermore, Example 22 below shows that a PKCα inhibitor effectively promoted wound healing in vivo thus indicating that the PKCα isoform may be antagonistic to wound healing.

PKCη has a unique tissue distribution. It is predominantly expressed in epithelial tissues (Osada et al., 1990; Chida et al., 1994). In situ hybridization studies as well as immunohistochemical studies have demonstrated that PKCη is highly expressed in the differentiating and differentiative layers (Osada et al., 1990). The results presented herein suggest the role of PKCη as a functional regulator of both proliferation and differentiation of skin depending on the cellular physiology. When keratinocytes are maintained in a proliferative state under low Ca²⁺ conditions, PKCη induced the proliferation rate five to seven times above control keratinocytes. However, when cells were induced to differentiate by elevating the Ca²⁺ concentration, differentiation was induced in a faster and higher rate in comparison to control cells (see Example 12). This could explain the ability of PKCη to dramatically induce wound healing and formation of granulation tissue as both proliferative capacity and formation of differentiation layers were achieved. Interestingly, the wound healing results in vivo and the expression of PKCη in embryonic tissue, which normally does not express PKCη at high levels in adulthood, would suggest a possible role for PKCη in the proliferation and tissue organization of other tissues as well. This includes neuronal as well as dermal and muscle tissue, which were efficiently healed in the granulation tissue of the wound. Furthermore, the ability to specifically regulate differentiation of keratinocytes and induce normal differentiation in actively proliferating cells by utilizing a dominant negative mutant, allows specifically manipulating differentiation and controlling hyperproliferative disorders involved in wound healing.

It is exemplified herein that the healing ability of PKCη is exerted in vivo on wounds that were produced on the backs of nude mice. Example 14 below shows that administration of PKCη expressing construct to the wound resulted in a granulation tissue formation, four days after topical infection.

Overall, the results presented herein demonstrate that modulating expression and/or activity of distinct PKC isoforms is an effective tool to affect wound healing. Accordingly, as shown by the inventors in previous application Ser. No. 10/169,801 and Ser. No. 10/644,775, wound healing may be promoted by enhancing the expression and/or activity of isoforms PKCδ, PKCη, PKC-ε and PKCζ, or by inhibiting the expression and/or activity of isoform PKCα, PKCβ or PKCη. Furthermore, it has been discovered by the inventors according to the present invention that the healing process of skin wounds can be efficiently induced or accelerated by modulating expression and/or activity of at least two PKC isoforms.

Thus, according to one aspect of the present invention, there is provided a method of inducing or accelerating a healing process of a damaged skin or skin wound, the method comprising modulating expression and/or activity of at least two PKC isoforms in skin cells colonizing the damaged skin or skin wound area, to thereby induce or accelerate the healing process of the damaged skin or skin wound.

In one embodiment of the invention, the method comprises the step of administering to the damaged skin or skin wound area therapeutically effective amounts of at least two PKC isoform modulating agents, each agent being capable of modulating the production and/or activity of each of said at least two PKC isoforms.

The invention further provides a pharmaceutical composition comprising a pharmaceutically acceptable carrier and a therapeutically effective amount of at least two agents, each of them capable of modulating the production and/or activity of one PKC isoform in the damaged skin or skin wound area.

The PKC isoform according to the invention includes PKC-α, PKC-β, PKC-δ, PKC-ε, PKC-η, PKC-ζ, PKC-γ, PKC-θ, PKC-λ and PKC-ι. In preferred embodiments, the PKC isoform is selected from the group consisting of PKC-α, PKC-β, PKC-δ, PKC-ε, PKC-η and PKC-ζ.

In one embodiment, at least one of said at least two PKC isoform modulating agents used in the methods or compositions of the invention is a PKC isoform inhibitor, preferably a PKC-α, PKC-β, PKC-η or PKC-ζ inhibitor, more preferably a PKC-α inhibitor or a PKC-η inhibitor.

Examples of PKC-α inhibitors that can be used according to the present invention include, without being limited to, a PKC-α pseudosubstrate inhibitor such as the PKC-α pseudosubstrate inhibiting peptides of SEQ IDS NO: 1-7, and a peptide binding to the substrate region such as the peptides of SEQ ID NO: 8 to NO. 24. The peptides of SEQ ID NO:1 to NO: 24 may be N-acylated, preferably by an acyl group derived from a C12-C20 fatty acid, more preferably C14 acyl (myristoyl). In one most preferred embodiment, the PKC-α inhibitor is the N-myristoylated PKC-α pseudosubstrate peptide of SEQ ID NO: 1 (herein sometimes designated HO/02).

Examples of PKC-η inhibitors that can be used according to the present invention include, without being limited to, a PKC-η pseudosubstrate inhibitor such as the N-myristoylated PKC-η pseudosubstrate inhibiting peptide of SEQ ID NO: 25, a PKC-η inhibitor that binds to the substrate region such as the peptides of SEQ ID NO: 26 and NO:27, and Copolymer 1, the active ingredient of the drug glatiramer acetate (Copaxone®, Teva Pharmaceutical Industries, Israel), clinically used for treating multiple sclerosis. Copolymer-1 was discovered in accordance with the present invention to be a specific and very effective PKCη inhibitor (see Example 29 hereinafter).

Examples of PKC-β inhibitors that can be used according to the present invention include, without being limited to, a PKC-β inhibitor that binds to the substrate region such as the peptides of SEQ ID NO: 28 to NO:38.

Examples of PKC-ζ inhibitors that can be used according to the present invention include, without being limited to, a PKC-ζ. inhibitor that binds to the substrate region such as the peptides of SEQ ID NO: 39 to NO:43.

According to another embodiment, at least one of said at least two PKC isoform modulating agents used in the method or composition of the invention is a PKC isoform activator, preferably a PKCδ activator, a PKCε activator and a PKCζ activator.

The PKC isoform activators for use in the methods and compositions of the present invention include, without being limited to, a peptide binding to a PKC isoform substrate region, a peptide acting on a PKC isoform phosphorylation site, insulin, a growth factor, bryostatin, a PKC isoform RACK peptide or a MARCKS (myristoylated alanine-rich C kinase substrate)-derived peptide.

In one embodiment, the PKC isoform is PKCδ and the PKCδ activator may be, without being limited to, insulin, a peptide binding to the PKCδ substrate region such as those of SEQ ID NO: 44 to NO: 51; a peptide acting on the PKC-δ phosphorylation site such as those of SEQ ID NO: 52 to NO: 54; a PKCδ RACK peptide, peptides corresponding to the C2 domain (disclosed in Benes et al., 2005). In a most preferred embodiment of the invention, the PKCδ activator is insulin.

In another embodiment, the PKC isoform is PKCζ and the PKCζ. activator is a PKCε RACK peptide.

In another embodiment, the PKC isoform is PKCζ and the PKCζ. activator is the PKC-ζ MARCKS-derived peptide of SEQ ID NO:55.

In certain embodiments of the invention, a growth factor is a PKC isoform activator without ascertaining which isoform is directly activated. In other embodiments, a growth factor can be used as a PKC isoform modulating agent without identification of the type of modulation and of the isoform affected by such modulation. Examples of growth factors useful in the methods and compositions of the invention include, without being limited to, platelet-derived growth factor (PDGF, preferably PDGF-BB), keratinocyte growth factor (KGF), epidermal growth factor (EGF), transforming growth factor-β (TGF-β), endothelial cell growth factor (ECGF) and insulin-like growth factor 1 (IGF1).

In other embodiments of the invention, some agents can be used as PKC isoform modulating agents without identification of the type of modulation and of the isoform affected by such modulation. Examples of such agents useful in the methods and compositions of the invention include, without being limited to, adipokines such as adipsin, adiponectin, apelin, visfatin, resistin, leptin, lipoprotein lipase, plasminogen activator inhibitor-1 (PAI-1), angiotensinogen, angiotensin I-IV and cycloanalogues thereof, 1-butyrylglycerol, matrix metalloproteinase 2, matrix metalloproteinase 9, vascular endothelial growth factor (VEGF), interleukin-6 (IL-6), IL-4, tumor necrosis factor alpha (TNF-α); a growth factor such as PDGF, KGF, EGF, TGF-β, ECGF or IGF1; Copolymer-1 or a peroxisome proliferator-activated receptor-γ (PPAR-γ) antagonist such as GW9662 (2-chloro-5-nitrobenzanilide).

Several combinations of PKC isoform modulating agents are possible and are encompassed by the present invention.

In one most preferred embodiment of the present invention, the method comprises inhibiting expression and/or activity of PKCα and activating expression and/or activity of PKCδ in skin cells colonizing the damaged skin or skin wound area. Any PKCα inhibitor and any PKCδ activator can be used according to the invention, but the most preferred formulation (herein designated sometimes HO/03/03) comprises the N-myristoylated PKCα pseudosubstrate peptide of SEQ ID NO: 1 as the PKCα inhibitor and insulin as the PKCδ activator (see Examples 22-26). In another preferred embodiment, the formulation contains the N-myristoylated PKCα pseudosubstrate peptide of SEQ ID NO: 1 as the PKCα inhibitor and the PKCδ RACK peptide as the PKCδ activator (Example 27).

The insulin for use in the present invention may be recombinant or from a natural source such as human insulin or a non-human mammal insulin that is suitable for human use such as porcine insulin.

In another preferred embodiment according to the invention, the method comprises inhibiting expression and/or activity of PKCη and activating expression and/or activity of PKCδ in skin cells colonizing the damaged skin or skin wound area. In preferred embodiments, the PKCη inhibitor is Copolymer-1 or the N-myristoylated PKCη pseudosubstrate peptide of SEQ ID NO: 25 and the PKCδ activator is insulin.

According to yet another preferred embodiment of the present invention, the method comprises inhibiting expression and/or activity of both PKCα and PKCη in skin cells colonizing the damaged skin or skin wound area. In preferred embodiments, the PKCα inhibitor is the N-myristoylated PKCα pseudosubstrate peptide of SEQ ID NO: 1 and the PKCη inhibitor is Copolymer-1 or the N-myristoylated PKCη pseudosubstrate peptide of SEQ ID NO: 25.

In yet a further preferred embodiment of the invention, the method comprises inhibiting expression and/or activity of PKCβ and activating expression and/or activity of PKCδ in skin cells colonizing the damaged skin or skin wound area. In preferred embodiments, the PKC-β inhibitor is a pseudosubstrate peptide of SEQ ID NO: 28 to 38 and the PKCδ activator is insulin. Also effective was a combination of PKCβ inhibition and an adipokine such as IL-6.

According to another preferred embodiment, the invention relates to the modulation of the expression and/or activity of three different PKC isoforms. In one preferred embodiment, the PKC isoforms are PKCα, δ, and η and the agents are the N-myristoylated peptide of SEQ ID NO: 1 (HO/02; PKCα inhibitor), insulin (PKCδ activator) and Copolymer-1 (PKCη inhibitor) (Example 31).

As will be described in detail hereinafter, there are alternative ways of modulating PKC expression and/or activity, some of them involving genetic manipulation with adenovirus constructs capable of modulating expression and/or activity of specific PKC isoforms. All these alternative methods are encompassed by the present invention. In preferred embodiments of the invention, inhibition of expression and/or activity of a PKC isoform is carried out by down-regulating expression and/or activity of the isoform, preferably using a dominant-negative (DN) PKC adenovirus construct and activation of expression and/or activity of a PKC isoform is carried out by upregulating expression and/or activity of the isoform, preferably using a wild type (WT) PKC adenovirus construct.

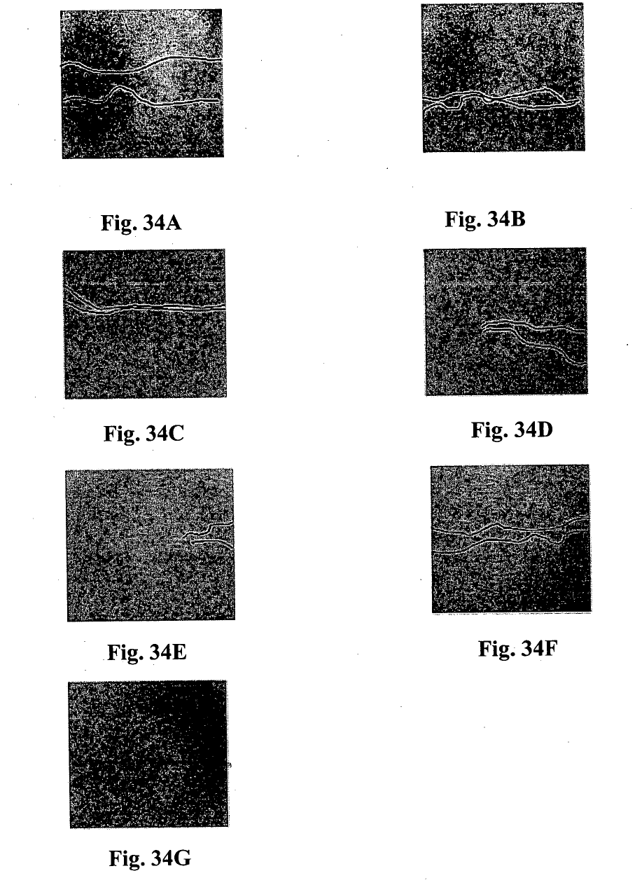

Thus, as shown in Example 28 and FIGS. 34-39, using DNPKC α, β and ζ adenovirus constructs and WTPKCδ, ε, η and ζ adenovirus constructs and several agents for modulation of at least two PKC isoforms expression and/or activity, the following results were obtained: (i) inhibition of PKCα expression and/or activity combined with administration of an adipokine such as adipsin, IL-6, TNFα, or a growth factor such as KGF or a PKCδ activator such as insulin or a PKCδ RACK peptide; (ii) inhibition of PKCα combined with inhibition of PKCη, inhibition of PKCε, activation of PKCδ, or activation of PKCζ; (iii) inhibition of PKCζ combined with administration of a growth factor such as KFG or an adipokine such as IL-6, TNFα or adiponectin; (iv) inhibition of PKCβ combined with activation of PKCδ, preferably by administration of insulin, or by modulation with a growth factor such as KGF, an adipokine such as IL-6, or a PPAR-γ antagonist such as GW9662; (v) inhibition of PKCα combined with the stimulation of PKCη, PKCε or PKCζ in the cells; (vi) activation of PKCδ activity and/or expression combined with activating PKCε, activating PKCζ, or inhibiting PKCα in the cells, or by administration of an adipokine such as adipsin.

In another aspect, the present invention provides a pharmaceutical composition for topical application for inducing or accelerating a healing process of a damaged skin or skin wound, the pharmaceutical composition comprising a pharmaceutically acceptable carrier and, as an active ingredient, a therapeutically effective amount of at least two PKC isoform modulating agents, each of them capable of modulating the expression and/or activity of one PKC isoform in the damaged skin or skin wound area. All the embodiments described above for the methods of the invention are preferred embodiments also for the pharmaceutical compositions of the invention.

The pharmaceutical compositions of the invention are both for human use and for veterinary use in the treatment of animal wounds.

As shown previously by the inventors, insulin can be directly administered to the wound. As described in Examples 21 and 22 hereinbelow, a topical application of insulin on wounds at a concentration ranging from 0.1-10 μM effectively promoted epidermal and dermal closure and subsequently wound healing. Yet, surprisingly and unexpectedly, the application of insulin combined with PDGF-BB growth factor or with a PKCα inhibitor, in accordance with the present invention, resulted in a substantial and synergistic improvement of the wound healing process over the insulin alone.

Thus, according to the present invention, a method of inducing or accelerating a healing process of a skin wound or damage comprises administering to the damaged skin or skin wound area a therapeutically effective amount of insulin (acting as PKCδ activator) and at least one additional agent acting in synergy with the insulin, so as to induce or accelerate the healing process of the skin wound or damage. Preferably, the agent is a PKCα inhibitor, most preferably the N-myristoylated peptide of SEQ ID NO:1. Further preferably, the agent is a growth factor such as PDGF, EGF, TGFβ, KGF, ECGF or IGF1, more preferably PDGF-BB.

The direct administration of insulin, either alone or combined with another agent, may be effected by a single or by repeat applications. While reducing the present invention to practice, the inventors surprisingly discovered that a treatment with a single application of insulin at a concentration of 1 μM was substantially more effective in healing wounds than with seven repeat daily applications of insulin at a similar concentration (see Example 20 below).

Thus, according to another aspect of the present invention, there is provided a method of inducing or accelerating a healing process of a skin wound or damage by administering to the skin wound a single dose-unit of a therapeutically effective amount of insulin. Preferably the single dose-unit comprises 0.001 to 5 μM, preferably 0.01 to 0.5 μM of insulin in, for example, an aqueous solution, gel, cream, paste, lotion, spray, suspension, powder, dispersion, salve or ointment formulation in an amount sufficient to cover a 1 cm area of the skin wound, e.g., 0.01-0.5 ml.

The timing of administering insulin onto wounds may be critical, as illustrated in Example 20 in the Examples section that follows. For example, a single application of insulin to a 4 day-old wound resulted in effective wound healing. Thus, according to another aspect of the present invention, there is provided a method of inducing or accelerating a healing process of an old skin wound by administering to the wound a single dose of a therapeutically effective amount of insulin.

The phrase “old skin wound” used herein refers to a skin wound that is at least one day old, at least two days old, at least three days old, preferably, at least four days old. Preferably, the wound should be treated at a critical stage in order to make the treatment effective for healing the wound

A pharmaceutical composition for inducing or accelerating a healing process of a skin wound or damage, according to another aspect of the present invention, includes, as an active ingredient, a therapeutically effective amount of insulin, at least one additional agent acting in synergy with the insulin, and a pharmaceutically acceptable carrier designed for topical application of the pharmaceutical composition. Preferably, the agent is a PKCα inhibitor or a growth factor such as PDGF, EGF, TGFβ, KGF, ECGF or IGF1. The pharmaceutically acceptable carrier can be adapted to provide a composition in the form of a gel, a cream, a paste, a lotion, a spray, a suspension, a powder, a dispersion, a salve and an ointment, as is further detailed hereinunder. Solid supports can also be used for prolonged release of insulin into the wound. It will be appreciated that the insulin can be native or preferably recombinant, of a human or any other suitable source.

According to another aspect of the present invention, a pharmaceutical composition for inducing or accelerating a healing process of a skin wound or damage, may include a single dose-unit of insulin selected capable of inducing or accelerating a healing process of the skin wound or damage, and a pharmaceutically acceptable carrier being designed for topical application of the pharmaceutical composition. Preferably, the single dose-unit of insulin is ranging from 0.001 to 5 μM, preferably 0.01 to 0.5 μM, in a 0.01-0.5 ml formulation dose-unit.

While reducing the present invention to practice, the present inventors surprisingly and unexpectedly uncovered that Copolymer-1 is capable of substantially promoting wound healing in vitro and in vivo (see Example 28 in the Examples section which follows). While copolymer-1 has been previously known as an immunomodulating agent used for treating multiple sclerosis and central nerve system disorders (U.S. Pat. Nos. 6,620,847, 6,362,161, 6,342,476, 6,054,430, 6,046,898, 5,981,589 and 5,800,808; U.S. application Ser. Nos. 10/615,865, 10/666,857 and 10/014,477), the prior art does not describe or suggest the use of Copolymer-1 for accelerating the process of wound healing.

Copolymer-1 is the active ingredient of the drug Copaxone® (Teva Pharmaceutical Industries Ltd., Petach Tikva, Israel) which is used clinically for treating multiple sclerosis. Copolymer-1 is a synthetic polypeptide analog of myelin basic protein (MBP), which is a natural component of the myelin sheath. Chemically, Copolymer-1 is a random copolymer of the 4 amino acids L-glutamic acid, L-alanine, L-lysine and L-tyrosine. In the form of its acetate salt it is known as glatiramer acetate and has an average molecular weight of 4,700-11,000 daltons. Copolymer-1 molecules of higher molecular weight are also known (MW 15,000-18,000) and can be used according to the invention.

Thus, according to another aspect of the present invention there is provided a method of inducing or accelerating a healing process of a damaged skin or skin wound, wherein the method is effected by administering to the damaged skin or skin wound area a therapeutically effective amount of copolymer-1, preferably at a concentration ranging between 1 to 500 μg/ml. A pharmaceutical composition for use in the method according to this aspect of the present invention therefore includes, as an active ingredient, a therapeutically effective amount of Copolymer-1 and a pharmaceutically acceptable carrier.

As described hereinbefore, Copolymer-1 was found to be a potent inhibitor of PKCη and can be used according to the invention with other PKC modulators, for example, with insulin (PKCδ activator), with the N-myristoylated peptide of SEQ ID NO:1 (PKCα inhibitor), or both.

Skin is not considered to be a classic insulin responsive tissue. Therefore, the effects of insulin in skin are mostly attributed to its ability to activate the closely related IGFR. It was shown that in keratinocytes, both insulin and IGF1 can stimulate both receptors and activate similar downstream effectors (Wertheimer et al., 2000). However, the present invention demonstrates that whereas both factors induce keratinocyte proliferation in a dose-dependent manner, each exerts its effects through distinct signaling pathways. The initial indication for differential regulation of keratinocyte proliferation by insulin and IGF1 was confirmed by the finding that these hormones had an additive effect on keratinocyte proliferation when added together, at maximal proliferation-inducing concentration of each hormone (see Example 15). In order to identify the divergence point in insulin and IGF1 signaling pathway in regulation of keratinocyte proliferation, elements known to both regulate keratinocyte proliferation and to act as downstream effectors of insulin signaling were examined. These studies revealed that insulin signaling is specifically mediated by PKCδ in keratinocyte proliferation (see Example 17). PKCδ is a unique isoform among the PKC family of proteins involved specifically in growth and maturation of various cell types (Gschwendt, 1999). However, while PKCδ was shown to be specifically regulated by stimulation of several growth factors including EGF, PDGF and neurotransmitters, its physiological effects were shown to participate in growth factor inhibition of cell growth including apoptosis, differentiation, and cell cycle retardation or arrest (Bajou et al., 1998; Alessenko et al., 1992; Soltoff and Toker, 1995; Mischak et al., 1993a; Sun et al., 1992; Mischak et al., 1993b). Recently it was shown that within 12-24 hours after elevation of Ca²⁺, a selective loss of the α6β4 integrin complex is linked to induction of the K1 in cultured mouse keratinocytes (Tennenbaum et al., 1996a). The loss of α6β4 protein expression is a consequence of transcriptional and post-translational events including enhanced processing of the α6 and β4 chains. In preliminary studies a link was established between the activation of PKC and the processing and regulation of the α6β4 integrin. These results are in agreement with previous results on the role of PKCδ as well as PKCζ in loss of α6β4 expression and hemidesmosome formation inducing keratinocyte detachment. However, the present invention identifies another role for PKCδ, as a target for insulin-induced keratinocyte proliferation. The examples below show that only insulin stimulation, but not a variety of growth factors, including, but not limited to, EGF, KGF, PDGF, ECGF and IGF1, can translocate and activate PKCδ, but not any of the other PKC isoforms expressed in skin. The importance of PKCδ to insulin stimulation was further confirmed when the mitogenic stimulation by EGF, KGF, PDGF, ECGF and IGF1 were not abrogated by the dominant negative mutant of PKCδ and insulin appeared to be the primary activator of this PKC isoform in the regulation of keratinocyte proliferation (see Example 17). However, when keratinocytes were infected with wild type (WT) PKCδ, keratinocytes mitogenic stimulation by EGF and KGF was enhanced. This suggests that PKCδ activation is also essential for the proliferative stimulation of other growth factors by upstream signaling pathways. Moreover, down stream elements were characterized which mediate in insulin induced PKCδ activation and keratinocyte proliferation and the involvement of STAT3, a transcriptional activator in this process, was identified. STAT (Signal Transducers and Activators of Transcription) proteins are a family of transcription factors recruited by a variety of cytokines and growth factors. Among the seven known STAT family members STAT3 is unique. Targeted disruption of STAT3 but not other STAT family members leads to early embryonic lethality. Specifically, when STAT3 was conditionally ablated in skin, skin remodeling was severely disrupted. Upon activation, STAT proteins form homo or heterodimers, translocate to the nucleus and bind to DNA response elements of target genes to induce transcription. It was found that in keratinocytes, PKCδ but not other PKC isoforms expressed in skin (PKCs α, ζ, η and ε) is constitutively associated with STAT3 (see Example 18). Furthermore, insulin regulates phosphorylation, activation and nuclear translocation of STAT3 via specific activation of PKCδ. Inhibition of PKCδ activity by a pharmacological inhibitor, rottlerin or by over-expressing a dominant negative PKCδ mutant abrogated insulin induced STAT3 activation and nuclear translocation. Finally, over-expression of a dominant negative PKCδ mutant inhibited keratinocyte proliferation induced by over-expression of STAT3 (see Example 18). These results suggest a role for insulin induced PKCδ activity in transcriptional activation by STAT3 in skin keratinocyte proliferation. As STAT3 is important for skin remodeling and is a down stream effector recruited by a variety of cytokines and growth factors, overall these results suggest PKCδ activation as a primary downstream element mediating the proliferation of keratinocytes. Specifically, PKCδ could be the primary candidate for the pathogenesis of defective wound healing as it appears in diabetic patients. The link between PKCδ and wound healing has also been corroborated in vivo. Utilizing a newly constructed PKCδ null mouse it is shown herein that the lack of PKCδ delays wound healing in mice skin (see Example 19). The link between PKCδ and insulin signaling has also been established in several other systems. For example, it was recently shown that in muscle cultures, PKCδ mediates insulin-induced glucose transport (Braiman et al., 1999a; Braiman et al., 1999b). Similarly, in cells over-expressing the insulin receptor, insulin stimulation was shown to be associated with activation of PKCδ (Bandyopadhyay et al., 1999; Formisano et al., 1998; Wang et al., 1999). However, whereas in these studies insulin mediated PKCδ activation has been linked to the metabolic effects of insulin, this is the first report linking PKCδ to insulin mediated cell proliferation. An identified dual role for PKCδ in regulation of both keratinocytes proliferation and the control of the early differentiation stages where cells lose their adherence to the underlying basement membrane was shown. This would suggest insulin induced PKCδ as a primary candidate of regulation of the physiological balance between proliferation and differentiation in skin.

Previous studies on the effects of distinct PKC isoforms in skin have been hampered as a result of the difficulty in introducing foreign genes efficiently into primary cells by conventional methods due to the short life span, differentiation potential and the inability to isolate stable transformants. To overcome these obstacles, viral vectors are being used to introduce genes of interest. Viral vectors are developed by modification of the viral genome in the form of replicative defective viruses. The most widely used viral vectors are the retroviruses and adenoviruses, which are used for experimental as well as gene therapy purposes (Kuroki et al., 1999). Specifically, the high efficiency of adenovirus infection in non replicating cells, the high titer of virus and the high expression of the transduced protein makes this system highly advantageous to primary cultures compared to retroviral vectors. As adenoviruses do not integrate into the host genome and the stable viral titers can be rendered replication deficient, these viral constructs are associated with minimal risk for malignancies in human as well as animal models (Rosenfeld et al., 1991). To date, in skin, adenovirus constructs have also been used successfully with high efficiency of infection with ex vivo and in vivo approaches (Setoguchi et al., 1994; Greenhalgh et al., 1994). An adenovirus vector, which was developed by I. Saito and his associates (Miyake et al., 1996) was used in the present study. The cosmid cassette (pAxCAwt) has nearly a full length adenovirus 5 genome but lacks E1A, E1B and E3 regions, rendering the virus replication defective. It contains a composite CAG promoter, consisting of the cytomegalovirus immediate-early enhancer, chicken β-actin promoter, and a rabbit β-globin polyadenylation signal, which strongly induces expression of inserted DNAs (Kuroki et al., 1999; Miyake et al., 1996). A gene of interest is inserted into the cosmid cassette, which is then co-transfected into human embryonic kidney 293 cells together with adenovirus DNA terminal protein complex (TPC). In 293 cells that express E1A and E1B regions, recombination occurs between the cosmid cassette and adenovirus DNA-TPC, yielding the desired recombinant virus at an efficiency one hundred fold that of conventional methods. Such high efficiency is mainly due to the use of the adenovirus DNA-TPC instead of proteinased DNA. Furthermore, the presence of longer homologous regions increases the efficiency of the homologous recombination. Regeneration of replication competent viruses is avoided due to the presence of multiple EcoT221 sites. It should be noted in this respect that keratinocytes were infected with distinct PKC recombinant adenoviruses and demonstrated 24 hours later effective over-expression of PKC isoforms (see Example 1).

Thus, another way by which modulating a PKC isoform expression and/or activation is effected according to the present invention is by inducing over-expression of a PKC isoform in the skin wound cells. This can be achieved by transforming the cells with a cis-acting element sequence integrated, by way of homologous recombination, upstream to an endogenous protein kinase C of the cells and thereby causing the cells to produce natural protein kinase C. A “cis-acting element” is used herein to describe a genetic region that serves as an attachment site for DNA-binding proteins (e.g., enhancers, operators and promoters), thereby affecting the activity of one or moregenes on the same chromosome.

Still alternatively, this can be achieved by transforming the cells with a recombinant protein kinase C gene, such as, but not limited to, PKC-β1 gene (Accession Nos. X06318, NM002738), PKC-β2 gene (Accession No. X07109), PKC-γ gene (Accession No. L28035), PKC-θ gene (Accession No. L07032), PKC-λ gene (Accession No. D28577), PKC-ι gene (Accession No. L18964), PKC-α gene (Accession No. X52479), PKC-δ gene (Accession Nos. L07860, L07861), PKC-ε gene (Accession No. X72974), PKC-η gene (Accession No. Z15108) and PKC-ζ gene (Accession Nos. Z15108, X72973, NM002744), and thereby causing the cells to produce recombinant protein kinase C.

A pharmaceutical composition for inducing or accelerating a healing process of a skin wound according to this aspect of the present invention therefore includes, as an active ingredient, a nucleic acid construct designed for transforming cells of the skin wound to produce a protein kinase C, and a pharmaceutically acceptable carrier designed for topical application of the pharmaceutical composition.

Still another way by which modulating PKC expression and/or activation is effected according to the present invention is by using a PKC activator, such as, but not limited to Ca²⁺, insulin or bryostatin 1, a peptide binding to the PKC isoform substrate region, a peptide acting on the PKC isoform phosphorylation site, a growth factor, a PKC isoform RACK peptide or a MARCKS (myristoylated alanine-rich C kinase substrate)-derived peptide.

as one of the at least two PKC modulating agents so as to induce or accelerate the healing process of the skin wound.

Still yet another way by which modulating PKC expression and/or activation is effected according to the present invention by downregulating expression and/or activity of at least one of the at least two PKC isoforms.

Downregulating activity of PKC isoform may be effected by a PKC isoform pseudosubstrate inhibitor such as, for example PKCα, PKCζ, PKC or PKCη pseudosubstrate inhibitors (CalbioChem, California USA), or another PKC isoform inhibitor such as, for example, the PKCβ inhibitor peptide LY379196 (Eli Lilly, USA) and PKCδ inhibitor rottlerin (CalbioChem, California USA).

Alternatively, downregulating activity of a PKC isoform may be effected by a dominant negative (DN) PKC adenovirus construct such as described in the Examples section hereinbelow.

Downregulating expression of a PKC isoform may be effected by a small interfering RNA (siRNA) molecule. RNA interference is a two step process. The first step, which is termed as the initiation step, input dsRNA is digested into 21-23 nucleotide (nt) small interfering RNAs (siRNA), probably by the action of Dicer, a member of the RNase III family of dsRNA-specific ribonucleases, which processes (cleaves) dsRNA (introduced directly or via a transgene or a virus) in an ATP-dependent manner. Successive cleavage events degrade the RNA to 19-21 bp duplexes (siRNA), each with 2-nucleotide 3′ overhangs [Hutvagner and Zamore Curr. Opin. Genetics and Development 12:225-232 (2002); and Bernstein Nature 409:363-366 (2001)].

In the effector step, the siRNA duplexes bind to a nuclease complex to from the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore Curr. Opin. Genetics and Development 12:225-232 (2002); Hammond et al., (2001) Nat. Rev. Gen. 2:110-119 (2001); and Sharp Genes. Dev. 15:485-90 (2001)]. Although the mechanism of cleavage is still to be elucidated, research indicates that each RISC contains a single siRNA and an RNase [Hutvagner and Zamore Curr. Opin. Genetics and Development 12:225-232 (2002)].

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC [Hammond et al., Nat. Rev. Gen. 2:110-119 (2001), Sharp Genes. Dev. 15:485-90 (2001); Hutvagner and Zamore Curr. Opin. Genetics and Development 12:225-232 (2002)]. For more information on RNAi see the following reviews Tuschl ChemBiochem. 2:239-245 (2001); Cullen Nat. Immunol. 3:597-599 (2002); and Brantl Biochem. Biophys. Act. 1575:15-25 (2002).

Synthesis of RNAi molecules suitable for use with the present invention can be effected as follows. First, the PKC isoform mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene. A suitable siRNA according to the present invention can be, for example, an siRNA capable of inhibiting PKCα expression such as any of the nucleic acid sequences set forth in SEQ ID NOs: 56-71.

Another agent capable of downregulating a PKC isoform is a DNAzyme molecule capable of specifically cleaving an mRNA transcript or DNA sequence of the PKC isoform. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262). A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al., DNAzymes of similar design directed against the human Urokinase receptor were recently observed to inhibit Urokinase receptor expression, and successfully inhibit colon cancer cell metastasis in vivo (Itoh et al., 20002, Abstract 409, Ann Meeting Am Soc Gen Ther www.asgt.org). In another application, DNAzymes complementary to bcr-abl oncogenes were successful in inhibiting the oncogenes expression in leukemia cells, and lessening relapse rates in autologous bone marrow transplant in cases of CML and ALL.

Downregulation of a PKC isoform can also be effected by using an antisense polynucleotide capable of specifically hybridizing with an mRNA transcript encoding the PKC isoform.

Design of antisense molecules which can be used to efficiently down-regulate a PKC isoform must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al., Blood 91: 852-62 (1998); Rajur et al., Bioconjug Chem 8: 935-40 (1997); Lavigne et al., Biochem Biophys Res Commun 237: 566-71 (1997) and Aoki et al., (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al., Biotechnol Bioeng 65: 1-9 (1999)].

Such algorithms have been successfully used to implement an antisense approach in cells. For example, the algorithm developed by Walton et al., enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16: 1374-1375 (1998)].

Several clinical trials have demonstrated safety, feasibility and activity of antisense oligonucleotides. For example, antisense oligonucleotides suitable for the treatment of cancer have been successfully used [Holmund et al., Curr Opin Mol Ther 1:372-85 (1999)], while treatment of hematological malignancies via antisense oligonucleotides targeting c-myb gene, p53 and Bcl-2 had entered clinical trials and had been shown to be tolerated by patients [Gerwitz Curr Opin Mol Ther 1:297-306 (1999)].

More recently, antisense-mediated suppression of human heparanase gene expression has been reported to inhibit pleural dissemination of human cancer cells in a mouse model [Uno et al., Cancer Res 61:7855-60 (2001)].

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Another agent capable of downregulating a PKC isoform is a ribozyme molecule capable of specifically cleaving an mRNA transcript encoding a PKC isoform. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. Angiozyme was the first chemically synthesized ribozyme to be studied in human clinical trials. Angiozyme specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. Heptazyme, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

The therapeutically/pharmaceutically active ingredients of the present invention can be administered to a wound per se, or in a pharmaceutical composition mixed with suitable carriers and/or excipients. Pharmaceutical compositions suitable for use in context of the present invention include those compositions in which the active ingredients are contained in an amount effective to achieve an intended therapeutic effect.

As used herein a “pharmaceutical composition” refers to a preparation of one or more of the active ingredients described herein, or physiologically acceptable salts or prodrugs thereof, with other chemical components such as traditional drugs, physiologically suitable carriers and excipients. The purpose of a pharmaceutical composition is to facilitate administration of a compound or cell to an organism. Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Hereinafter, the phrases “physiologically suitable carrier” and “pharmaceutically acceptable carrier” are interchangeably used and refer to a carrier or a diluent that does not cause significant irritation to an organism and does not abrogate the biological activity and properties of the administered conjugate.

Herein the term “excipient” refers to an inert substance added to a pharmaceutical composition to further facilitate processes and administration of the active ingredients. Examples, without limitation, of excipients include calcium carbonate, calcium phosphate, various sugars and types of starch, cellulose derivatives, gelatin, vegetable oils and polyethylene glycols.

Techniques for formulation and administration of active ingredients may be found in “Remington's Pharmaceutical Sciences,” Mack Publishing Co., Easton, Pa., latest edition, which is incorporated herein by reference.

While various routes for the administration of active ingredients are possible, and were previously described, for the purpose of the present invention, the topical route is preferred, and is assisted by a topical carrier. The topical carrier is one, which is generally suited for topical active ingredients administration and includes any such materials known in the art. The topical carrier is selected so as to provide the composition in the desired form, e.g., as a liquid or non-liquid carrier, lotion, cream, paste, gel, powder, ointment, solvent, liquid diluent, drops and the like, and may be comprised of a material of either naturally occurring or synthetic origin. It is essential, clearly, that the selected carrier does not adversely affect the active agent or other components of the topical formulation, and which is stable with respect to all components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like. Preferred formulations herein are colorless, odorless ointments, liquids, lotions, creams and gels.

Ointments are semisolid preparations, which are typically based on petrolatum or other petroleum derivatives. The specific ointment base to be used, as will be appreciated by those skilled in the art, is one that will provide for optimum active ingredients delivery, and, preferably, will provide for other desired characteristics as well, e.g., emolliency or the like. As with other carriers or vehicles, an ointment base should be inert, stable, nonirritating and nonsensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight; again, reference may be made to Remington: The Science and Practice of Pharmacy for further information.

Lotions are preparations to be applied to the skin surface without friction, and are typically liquid or semi liquid preparations, in which solid particles, including the active agent, are present in a water or alcohol base. Lotions are usually suspensions of solids, and may comprise a liquid oily emulsion of the oil-in-water type. Lotions are preferred formulations herein for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like.

Creams containing the selected active ingredients are, as known in the art, viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington, supra, is generally a nonionic, anionic, cationic or amphoteric surfactant.

Gel formulations are preferred for application to the scalp. As will be appreciated by those working in the field of topical active ingredients formulation, gels are semisolid, suspension-type systems. Single-phase gels contain organic macromolecules distributed substantially uniformly throughout the carrier liquid, which is typically aqueous, but also, preferably, contains an alcohol and, optionally, an oil.

Carriers for nucleic acids include, but are not limited to, liposomes including targeted liposomes, nucleic acid complexing agents, viral coats and the like. However, transformation with naked nucleic acids may also be used.

Various additives, known to those skilled in the art, may be included in the topical formulations of the invention. For example, solvents may be used to solubilize certain active ingredients substances. Other optional additives include skin permeation enhancers, opacifiers, anti-oxidants, gelling agents, thickening agents, stabilizers, and the like.

As has already been mentioned hereinabove, topical preparations for the treatment of wounds according to the present invention may contain other pharmaceutically active agents or ingredients, those traditionally used for the treatment of such wounds. These include immunosuppressants, such as cyclosporine, antimetabolites, such as methotrexate, corticosteroids, vitamin D and vitamin D analogs, vitamin A or its analogs such as etretinate, tar, coal tar, anti pruritic and keratoplastic agents, such as cade oil, keratolytic agents, such as salicylic acid, emollients, lubricants, antiseptics and disinfectants, such as the germicide dithranol (also known as anthralin), photosensitizers such as psoralen and methoxsalen and UV irradiation. Other agents may also be added, such as antimicrobial agents, antifungal agents, antibiotics and anti-inflammatory agents. Treatment by oxygenation (high oxygen pressure) may also be co-employed.

The topical compositions of the present invention may also be delivered to the skin using conventional dermal-type patches or articles, wherein the active ingredients composition is contained within a laminated structure that serves as a drug delivery device to be affixed to the skin. In such a structure, the active ingredients composition is contained in a layer, or “reservoir”, underlying an upper backing layer. The laminated structure may contain a single reservoir, or it may contain multiple reservoirs. In one embodiment, the reservoir comprises a polymeric matrix of a pharmaceutically acceptable contact adhesive material that serves to affix the system to the skin during active ingredients delivery. Examples of suitable skin contact adhesive materials include, but are not limited to, polyethylenes, polysiloxanes, polyisobutylenes, polyacrylates, polyurethanes, and the like. The particular polymeric adhesive selected will depend on the particular active ingredients, vehicle, etc., i.e., the adhesive must be compatible with all components of the active ingredients-containing composition. Alternatively, the active ingredients-containing reservoir and skin contact adhesive are present as separate and distinct layers, with the adhesive underlying the reservoir which, in this case, may be either a polymeric matrix as described above, or it may be a liquid or hydrogel reservoir, or may take some other form.

The backing layer in these laminates, which serves as the upper surface of the device, functions as the primary structural element of the laminated structure and provides the device with much of its flexibility. The material selected for the backing material should be selected so that it is substantially impermeable to the active ingredients and to any other components of the active ingredients-containing composition, thus preventing loss of any components through the upper surface of the device. The backing layer may be either occlusive or nonocclusive, depending on whether it is desired that the skin become hydrated during active ingredients delivery. The backing is preferably made of a sheet or film of a preferably flexible elastomeric material. Examples of polymers that are suitable for the backing layer include polyethylene, polypropylene, and polyesters.

During storage and prior to use, the laminated structure includes a release liner. Immediately prior to use, this layer is removed from the device to expose the basal surface thereof, either the active ingredients reservoir or a separate contact adhesive layer, so that the system may be affixed to the skin. The release liner should be made from an active ingredients/vehicle impermeable material.

Such devices may be fabricated using conventional techniques, known in the art, for example by casting a fluid admixture of adhesive, active ingredients and vehicle onto the backing layer, followed by lamination of the release liner. Similarly, the adhesive mixture may be cast onto the release liner, followed by lamination of the backing layer. Alternatively, the active ingredients reservoir may be prepared in the absence of active ingredients or excipient, and then loaded by “soaking” in an active ingredients/vehicle mixture.

As with the topical formulations of the invention, the active ingredients composition contained within the active ingredients reservoirs of these laminated systems may contain a number of components. In some cases, the active ingredients may be delivered “neat,” i.e., in the absence of additional liquid. In most cases, however, the active ingredients will be dissolved, dispersed or suspended in a suitable pharmaceutically acceptable vehicle, typically a solvent or gel. Other components, which may be present, include preservatives, stabilizers, surfactants, and the like.

The pharmaceutical compositions herein described may also comprise suitable solid or gel phase carriers or excipients. Examples of such carriers or excipients include, but are not limited to, calcium carbonate, calcium phosphate, various sugars, starches, cellulose derivatives, gelatin and polymers such as polyethylene glycols.

Dosing is dependent on the type, the severity and manifestation of the affliction and on the responsiveness of the subject to the active ingredients, as well as the dosage form employed, the potency of the particular conjugate and the route of administration utilized. Persons of ordinary skill in the art can easily determine optimum dosages, dosing methodologies and repetition rates. The exact formulation, route of administration and dosage can be chosen by the individual physician in view of the patient's condition (see e.g., Fingl, et al., 1975, in “The Pharmacological Basis of Therapeutics”, Ch. 1 p. 1).

Thus, depending on the severity and responsiveness of the condition to be treated, dosing can be a single or repetitive administration, with course of treatment lasting from several days to several weeks or until cure is effected or diminution of the skin lesion is achieved.

In some aspects the present invention utilizes in vivo and ex vivo (cellular) gene therapy techniques, which involve cell transformation and gene knock-in type transformation. Gene therapy as used herein refers to the transfer of genetic material (e.g. DNA or RNA) of interest into a host to treat or prevent a genetic or acquired disease or condition or phenotype. The genetic material of interest encodes a product (e.g., a protein, polypeptide, peptide, functional RNA, antisense RNA) whose production in vivo is desired. For example, the genetic material of interest can encode a hormone, receptor, enzyme, polypeptide or peptide of therapeutic value. For review see, in general, the text “Gene Therapy” (Advanced in Pharmacology 40, Academic Press, 1997).

Two basic approaches to gene therapy have evolved (1) ex vivo; and (ii) in vivo gene therapy. In ex vivo gene therapy cells are removed from a patient or are derived from another source, and while being cultured are treated in vitro. Generally, a functional replacement gene is introduced into the cell via an appropriate gene delivery vehicle/method (transfection, transduction, homologous recombination, etc.) and an expression system as needed and then the modified cells are expanded in culture and returned to the host/patient. These genetically reimplanted cells have been shown to express the transfected genetic material in situ.

In in vivo gene therapy, target cells are not removed from the subject rather the genetic material to be transferred is introduced into the cells of the recipient organism in situ that is within the recipient. In an alternative embodiment, if the host gene is defective, the gene is repaired in situ (Culver, 1998. (Abstract) Antisense DNA & RNA based therapeutics, February 1998, Coronado, Calif.). These genetically altered cells have been shown to express the transfected genetic material in situ.

The gene expression vehicle is capable of delivery/transfer of heterologous nucleic acid into a host cell. The expression vehicle may include elements to control targeting, expression and transcription of the nucleic acid in a cell selective manner as is known in the art. It should be noted that often the 5'UTR and/or 3'UTR of the gene might be replaced by the 5'UTR and/or 3'UTR of the expression vehicle. Therefore, as used herein the expression vehicle may, as needed, not include the 5'UTR and/or 3'UTR of the actual gene to be transferred and only include the specific amino acid coding region.

The expression vehicle can include a promoter for controlling transcription of the heterologous material and can be either a constitutive or inducible promoter to allow selective transcription. Enhancers that may be required to obtain necessary transcription levels can optionally be included. Enhancers are generally any nontranslated DNA sequence, which works contiguously with the coding sequence (in cis) to change the basal transcription level dictated by the promoter. The expression vehicle can also include a selection gene as described herein below.

Vectors can be introduced into cells or tissues by any one of a variety of known methods within the art. Such methods can be found generally described in Sambrook et al., Molecular Cloning: A Laboratory Manual, Cold Springs Harbor Laboratory, New York 1989, 1992), in Ausubel et al., Current Protocols in Molecular Biology, John Wiley and Sons, Baltimore, Md. 1989), Chang et al., Somatic Gene Therapy, CRC Press, Ann Arbor, Mich. 1995), Vega et al., Gene Targeting, CRC Press, Ann Arbor Mich. (995), Vectors: A Survey of Molecular Cloning Vectors and Their Uses, Butterworths, Boston Mass. 1988) and Gilboa et al., (Biotechniques 4 (6): 504-512, 1986) and include, for example, stable or transient transfection, lipofection, electroporation and infection with recombinant viral vectors. In addition, see U.S. Pat. No. 4,866,042 for vectors involving the central nervous system and also U.S. Pat. Nos. 5,464,764 and 5,487,992 for positive-negative selection methods.

Introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

A specific example of DNA viral vector introducing and expressing recombination sequences is the adenovirus-derived vector Adenop53TK. This vector expresses a herpes virus thymidine kinase (TK) gene for either positive or negative selection and an expression cassette for desired recombinant sequences. This vector can be used to infect cells that have an adenovirus receptor, which includes most tissues of epithelial origin as well as others. This vector as well as others that exhibit similar desired functions can be used to treat a mixed population of cells and can include, for example, in vitro or ex vivo culture of cells, a tissue or a human subject.

Features that limit expression to particular cell types can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retroviruses, and is the process by which a single infected cell produces many progeny virions that bud off and infect neighboring cells. The result is that a large area becomes rapidly infected, most of which was not initially infected by the original viral particles. This is in contrast to vertical-types of infections, in which the infectious agent spreads only through daughter progeny. Viral vectors can also be produced that are unable to spread laterally. This characteristic can be useful if the desired purpose is to introduce a specified gene into only a localized number of targeted cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. The vector to be used in the methods and compositions of the invention will depend on desired cell type to be targeted and will be known to those skilled in the art.

Retroviral vectors can be constructed to function either as infectious particles or to undergo only a single initial round of infection. In the former case, the genome of the virus is modified so that it maintains all the necessary genes, regulatory sequences and packaging signals to synthesize new viral proteins and RNA. Once these molecules are synthesized, the host cell packages the RNA into new viral particles which are capable of undergoing further rounds of infection. The vector's genome is also engineered to encode and express the desired recombinant gene. In the case of non-infectious viral vectors, the vector genome is usually mutated to destroy the viral packaging signal that is required to encapsulate the RNA into viral particles. Without such a signal, any particles that are formed will not contain a genome and therefore cannot proceed through subsequent rounds of infection. The specific type of vector will depend upon the intended application. The actual vectors are also known and readily available within the art or can be constructed by one skilled in the art using well-known methodology.

The recombinant vector can be administered in several ways. If viral vectors are used, for example, the procedure can take advantage of their target specificity and consequently, do not have to be administered locally at the diseased site. However, local administration can provide a quicker and more effective treatment.

Procedures for in vivo and ex vivo cell transformation including homologous recombination employed in knock-in procedures are set forth in, for example, U.S. Pat. Nos. 5,487,992, 5,464,764, 5,387,742, 5,360,735, 5,347,075, 5,298,422, 5,288,846, 5,221,778, 5,175,385, 5,175,384, 5,175,383, 4,736,866 as well as Burke and Olson, Methods in Enzymology, 194:251-270 1991); Capecchi, Science 244:1288-1292 1989); Davies et al., Nucleic Acids Research, 20 (11) 2693-2698 1992); Dickinson et al., Human Molecular Genetics, 2(8): 1299-1302 1993); Duff and Lincoln, “Insertion of a pathogenic mutation into a yeast artificial chromosome containing the human APP gene and expression in ES cells”, Research Advances in Alzheimer's Disease and Related Disorders, 1995; Huxley et al., Genomics, 9:742-750 1991); Jakobovits et al., Nature, 362:255-261 1993); Lamb et al., Nature Genetics, 5: 22-29 1993); Pearson and Choi, Proc. Natl. Acad. Sci. USA 1993). 90:10578-82; Rothstein, Methods in Enzymology, 194:281-301 1991); Schedl et al., Nature, 362: 258-261 1993); Strauss et al., Science, 259:1904-1907 1993). Further, Patent Applications WO 94/23049, WO93/14200, WO 94/06908, WO 94/28123 also provide information.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above description illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A Laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al., (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al., (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Material and Experimental Methods

(i) Materials.

Tissue culture media and serum were purchased from Biological Industries (Beit HaEmek, Israel). Enhanced Chemical Luminescence (ECL) was performed with a kit purchased from BioRad (Israel). Monoclonal anti p-tyr antibody was purchased from Upstate Biotechnology Inc. (Lake Placid, N.Y., USA). Polyclonal and monoclonal antibodies to PKC isoforms were purchased from Santa Cruz (California, USA) and Transduction Laboratories (Lexington, Ky.). The α6 rat antimouse mAb (GoH3) was purchased from Pharmingen (San Diego, Calif.). The antibody 6844 for the α6A cytoplasmic domain was a gift from Dr. V. Quaranta (Scripps Research Institute, La Jolla, Calif.). The rat mAb directed against the extracellular domain of mouse β4 (346-11A) was a gift from Dr. S. J. Kennel (Oak Ridge National Laboratory, Oak Ridge, Tenn.). Rat mAB to phosphotyrosine was purchased from Sigma (St. Louis, Mo.) and rabbit anti phosphoserine was purchased from Zymed (San Francisco, Calif.). Horseradish peroxidase-anti-rabbit and anti-mouse IgG were obtained from Bio-Rad (Israel). Leupeptin, aprotinin, PMSF, DTT, Na-orthovanadate, and pepstatin were purchased from Sigma Chemicals (St. Louis, Mo.). Insulin (humulinR-recombinant human insulin) was purchased from Eli Lilly France SA (Fergersheim, France). IGF1 was a gift from Cytolab (Israel). Keratin 14 antibody was purchased from Babco-Convance (Richmond, Calif.) BDGF-BB was purchased from R&D Systems (Minneapolis) and PKCα pseudosubstrate myristoylated peptide was purchased from CalbioChem (San Diego, Calif.).

(ii) Isolation and Culture of Murine Keratinocytes.

Primary keratinocytes were isolated from newborn skin as previously described (Dlugosz et al., 1995). Keratinocytes were cultured in Eagle's Minimal Essential Medium (EMEM) containing 8% Chelex (Chelex-100, BioRad)-treated fetal bovine serum. To maintain a proliferative basal cell phenotype, the final Ca²⁺ concentration was adjusted to 0.05 mM. Experiments were performed 5-7 days after plating.

(iii) Preparation of Cell Extracts and Western Blot Analysis.

For crude membrane fractions, whole cell lysates were prepared by scraping cells into PBS containing 10 μg/ml aprotinin, 10 μg/ml leupeptin, 2 μg/ml pepstatin, 1 mM PMSF, 10 mM EDTA, 200 μM NaVO₄ and 10 mM NaF. After homogenization and 4 freeze/thaw cycles, lysates were spun down at 4° C. for 20 minutes in a microcentrifuge at maximal speed. The supernatant containing the soluble cytosol protein fraction was transferred to another tube. The pellet was resuspended in 250 μl PBS containing 1% Triton X-100 with protease and phosphatase inhibitors, incubated for 30 minutes at 4° C. and spun down in a microcentrifuge at maximal speed at 4° C. The supernatant contains the membrane fraction. Protein concentrations were measured using a modified Lowery assay (Bio-Rad DC Protein Assay Kit). Western blot analysis of cellular protein fractions was carried out as described (Tennenbaum et al., 1996).

(iv) Preparation of Cell Lysates for Immunoprecipitation.

Culture dishes containing keratinocytes were washed with Ca²⁺/Mg²⁺-free PBS. Cells were mechanically detached in RIPA buffer (50 mM Tris-HCl pH 7.4; 150 mM NaCl; 1 mM EDTA; 10 mM NaF; 1% Triton ×100; 0.1% SDS, 1% Na deoxycholate) containing a cocktail of protease and phosphatase inhibitors (20 μg/ml leupeptin; 10 μg/ml aprotinin; 0.1 mM PMSF; 1 mM DTT; 200 μM orthovanadate; 2 μg/ml pepstatin). The preparation was centrifuged in a microcentrifuge at maximal speed for 20 minutes at 4° C. The supernatant was used for immunoprecipitation.

(v) Immunoprecipitation.

The lysate was precleared by mixing 300 μg of cell lysate with 25 μl of Protein A/G Sepharose (Santa Cruz, Calif., USA), and the suspension was rotated continuously for 30 minutes at 4° C. The preparation was then centrifuged at maximal speed at 4° C. for 10 minutes, and 30 μl of A/G Sepharose was added to the supernatant along with specific polyclonal or monoclonal antibodies to the individual antigens (dilution 1:100). The samples were rotated overnight at 4° C. The suspension was then centrifuged at maximal speed for 10 minutes at 4° C., and the pellet was washed with RIPA buffer. The suspension was again centrifuged at 15,000×g (4° C. for 10 minutes) and washed four times in TBST. Sample buffer (0.5 M Tris.HCl pH 6.8; 10% SDS; 10% glycerol; 4% 2-beta-mercaptoethanol; 0.05% bromophenol blue) was added and the samples were boiled for 5 minutes and then subjected to SDS-PAGE.

(vi) Attachment Assays.

Twenty four-well petri plates (Greiner) were coated (250 μl/well) with 20 μg/ml of matrix proteins in PBS for 1 hour at 37° C. Following incubation, plates were washed and incubated with 0.1% BSA for 30 minutes at room temperature to block nonspecific binding. Keratinocytes cultures were trypsinized briefly with 0.25% trypsin and following detachment, cells were resuspended and keratinocytes (1×10⁶) added to the coated wells and incubated for 1 hour at 37° C. Nonadherent cells were removed, the wells were rinsed twice with PBS and the remaining cells were extracted in 1 M NaOH. Cell count was determined by protein concentrations using a modified Lowery assay (Bio-Rad DC Protein Assay Kit). Results were calculated by percentage relative to untreated controls.

(vii) Immunofluorescence.

Primary keratinocytes were plated on laminin 5 coated glass slides. Two days old keratinocytes were infected with PKC adenovirus for one hour, washed twice with PBS and maintained in culture in low Ca²⁺ MEM. 24 hours post infection; cells were fixed in 4% paraformaldehyde for 30 minutes followed by permeabilization with 0.2% Triton for 5 minutes. For analysis, control and PKC infected keratinocytes were rinsed with PBS and incubated overnight at 4° C. with PKC antibodies (Santa Cruz) diluted in 1% BSA in PBS. After incubation, slides were washed twice for 10 minutes with PBS and incubated with biotinylated secondary anti rabbit antibody for 20 minutes, washed twice in PBS and incubated with Streptavidin-FITC for 20 minutes. For analysis of α6β4 staining, glass slides were treated with 0.2% Triton X-100 for 5 minutes on ice followed by 5 minutes fixation in methanol. The slides were incubated with anti α6 or anti β4 antibodies overnight followed by incubation with biotinylated secondary anti rat antibody, respectively, for 20 minutes, washed twice in PBS and incubated with Streptavidin-FITC for 20 minutes. Following two washes in PBS, slides were mounted with glycerol buffer containing 1% of p-phenylenediamine (Sigma) and fluorescence examined by laser scanning confocal imaging microscopy (MRC1024, Bio-Rad, UK).

(viii) Adenovirus Constructs.

The recombinant adenovirus vectors were constructed as previously described (Ohba et al., 1998). The dominant negative mutants of mouse PKCs were generated by substitution of the lysine residue at the ATP binding site with alanine. The mutant cDNA was cut from SRD expression vector with EcoR I and ligated into the pAxCA1w cosmid cassette to construct the Ax vector. The dominant negative activity of the genes was demonstrated by the abrogation of its autophosphorylation activity.

(ix) Transduction of Keratinocytes with PKC Isoform Genes.

The culture medium was aspirated and keratinocyte cultures were infected with the viral supernatant containing PKC recombinant adenoviruses for one hour. The cultures were then washed twice with MEM and re-fed. Ten hours post-infection cells were transferred to serum-free low Ca²⁺-containing MEM for 24 hours. Keratinocytes from control and insulin-treated or IGF1-treated cultures were used for proliferation assays, ⁸⁶Rb uptake, or extracted and fractionated into cytosol and membrane fractions for immunoprecipitation, immunofluorescence and Western blotting as described.

(x) PKC Activity.

Specific PKC activity was determined in freshly prepared immunoprecipitates from keratinocyte cultures following appropriate treatments. These lysates were prepared in RIPA buffer without NaF. Activity was measured with the use of the SignaTECT Protein Kinase C Assay System (Promega, Madison, Wis., USA) according to the manufacturer's instructions. PKCα pseudosubstrate was used as the substrate in these studies.

(xi) Cell Proliferation.

Cell proliferation was measured by [³H]thymidine incorporation in 24-well plates. Cells were pulsed with [³H]thymidine (1 μCi/ml) overnight. After incubation, cells were washed five times with PBS and 5% TCA was added into each well for 30 minutes. The solution was removed and cells were solubilized in 1% Triton X-100. The labeled thymidine incorporated into cells was counted in a ³H-window of a Tricarb liquid scintillation counter.

(xii) Na⁺/K⁺ Pump Activity.

Na⁺/K⁺ pump activity was determined by the measurements of ouabain-sensitive uptake of ⁸⁶Rb by whole cells in 1 ml of K⁺-free PBS containing 2 mM RbCl and 2.5 μCi of ⁸⁶Rb. Rb uptake was terminated after 15 minutes by aspiration of the medium, after which the cells were rinsed rapidly four times in cold 4° C. K⁺-free PBS and solubilized in 1% Triton X-100. The cells from the dish were added to 3 ml H₂O in a scintillation vial. Samples were counted in a ³H-window of a Tricarb liquid scintillation counter. Rb-uptake specifically related to Na⁺/K⁺ pump activity was determined by subtraction of the cpm accumulated in the presence of 10⁻⁴ M ouabain from the uptake determined in the absence of the inhibitor.

(xiii) PKC Immunokinase Assay.

Purified and standardized PKC isozymes were kindly supplied by Dr. P. Blumberg (NCI, NIH, U.S.) and Dr. Marcello G. Kazanietz (University of Pennsylvania, School of Medicine). Primary keratinocytes were harvested in 500 μl 1% Triton Lysis Buffer (1% Triton-X 100, 10 μg/ml aprotinin and leupeptin, 2 μg/ml pepstatin, 1 mM PMSF, 1 mM EDTA, 200 μM Na₂VO₄, 10 mM NaF in 1×PBS). Lysates were incubated at 4° C. for 30 minutes, and spun at 16,000×g for 30 minutes at 4° C. Supernatants were transferred to a fresh tube. Immunoprecipitation of cell lysates was carried out overnight at 4° C. with 5 μg/sample anti-α6/GoH3 (PharMingen) and 30 μl/sample of protein A/G-Plus agarose slurry (Santa Cruz). Beads were washed once with RIPA buffer and twice with 50 mM Tris/HCl pH 7.5. 35 μl of reaction buffer (1 mM CaCl₂, 20 mM MgCl₂, 50 mM Tris.HCl pH 7.5) was added to each assay. To each assay, 5.5 μl/assay of a suspension of phospholipid vesicles containing either DMSO or 10 mM TPA was added to the slurry together with a standardized amount of specific PKC isozyme. The reaction was initiated by adding 10 μl/assay 125 mM ATP (1.25 μCi/assay [γ-32P] ATP, Amersham) and allowed to continue for 10 minutes at 30° C. The beads were then washed twice with RIPA buffer. 30 μl/sample protein loading dye (3×Laemmli, 5% SDS) was added and the samples were boiled for 5 minutes in a water bath. Proteins were separated by SDS-PAGE on a 8.5% gel, transferred onto Protran membranes (Schleicher & Schuell) and visualized by autoradiography. Phosphorylation of histones and phosphorylation of PKC substrate peptide were used as controls for PKC activity.

(xiv) In Vivo Wound Healing Assessment.

Our method and assessment of wound healing in vivo includes the infliction of full thickness skin incisions (20 mm) on the upper back of anesthetized mice and rats. The wounds are covered with a 22×6 mm surgical dressing. Following incision, wounds are treated once daily by application of tested compounds such as: insulin, PKCα pseudosubstrate inhibitor, Copolymer-1, growth factors, PKC modulating agents and various specific combinations. Treatments, 0.2-0.5 ml, are applied directly to the surgical dressing. Morphological evaluation is performed during the experiment with final assessment of parameters at the termination day. The animals are euthanized and biopsies are collected at critical time points: 1-30 days post wounding. The tissues are fixed in 4% paraformaldehide and paraffin blocks are prepared. The evaluation of wound healing is performed utilizing histological and immunohistochemical parameters. Epidermal closure is assessed by keratin 14 (K14) immunostaining, epidermal migration and differentiation by keratin 6 (K6) and keratin 1 (K1) immunostaining, respectively. We have also followed granulation tissue formation by proliferating cell nuclear antigen (PCNA) and collagen fiber distribution (Masson Tri-chrom). In addition, dermal contraction and remodeling are assessed by collagen fibers and hair follicles formation. Inflammation is assessed utilizing hematoxylin-eosin (H&E) and myeloperoxidase staining. Late stages of healing are assessed according to scar tissue formation and hypodermis regeneration utilizing H&E histostaining.

### Example 1

### Effective Over-Expression of PKC Isoforms Utilizing Recombinant Adenovirus Vectors

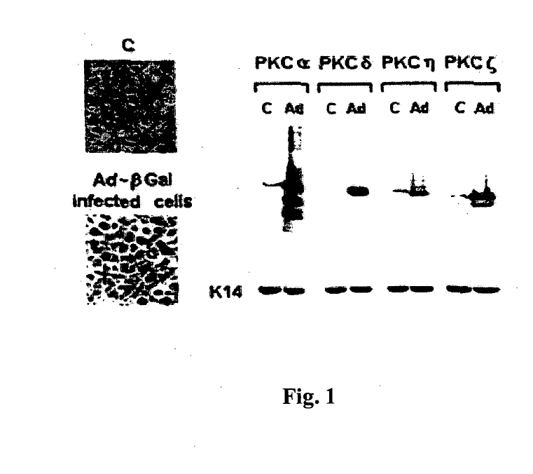

By utilizing a recombinant β-galactosidase adenovirus a high infection rate was achieved with more then 90% of the cultured keratinocyte population expressing the recombinant protein. The recombinant β-galactosidase adenovirus infection did not affect cell viability or cell growth. Furthermore, β-galactosidase expression was sustained for up to two weeks of culture and was used as a control infection in following experiments. The efficiency of recombinant PKC adenovirus constructs to induce protein expression and be activated properly in mouse keratinocyte cultures was examined. As seen by Western blotting in FIG. 1, 24 hours following a 1 hour infection with recombinant PKC adenovirus constructs, a dramatic increase in specific PKC protein expression was observed five to ten fold above the endogenous expression levels of the specific isoforms. Recombinant protein could be detected in infected keratinocyte cultures as early as 6 hours following infection and peak expression was obtained by 24 hours. Protein expression was sustained throughout the culture period (up to fourteen days).

### Example 2

### Over-Expressed PKC Isoforms are Activated by PKC Activators

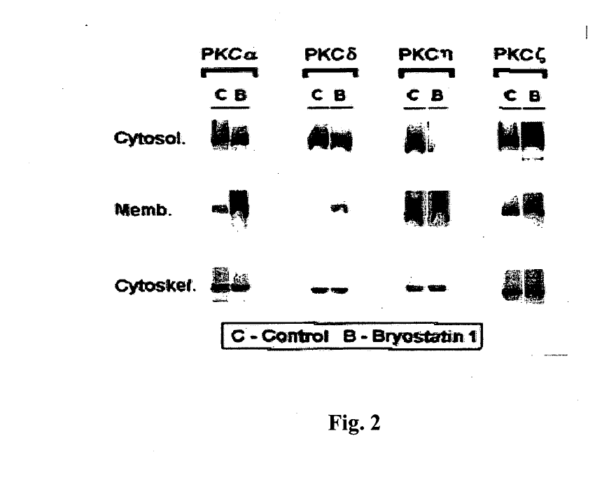

Recombinant proteins of the PKC isoforms responded typically to PKC activators. As seen in FIG. 2, treatment with bryostatin 1 (10 nM) induced translocation of PKCα and δ proteins to the membrane fraction, with a lesser effect on PKCη and ζ isoforms, similarly to results obtained with the endogenous isoforms and as expected from their cofactor requirements.

### Example 3

### Over-Expressed PKC Isoforms are Active in their Native Form

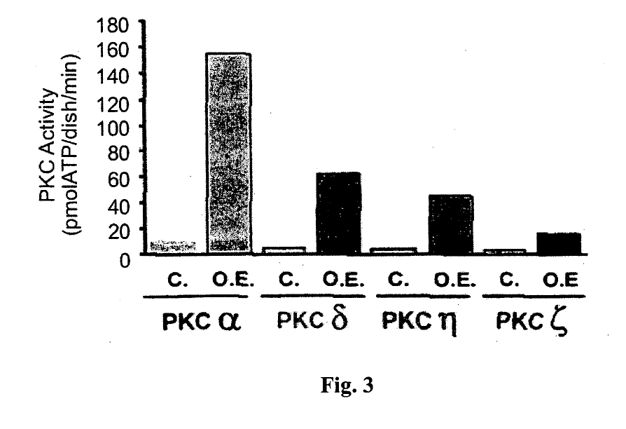

As early as 18 hours following infection, PKC kinase assays revealed that immunoprecipitates of distinct PKC isoforms were enzymatically active without further need of stimulation by PKC activators (FIG. 3).

### Example 4

### Over-Expression of Specific PKC Isoforms Induces Distinct Morphological Changes in Primary Keratinocytes

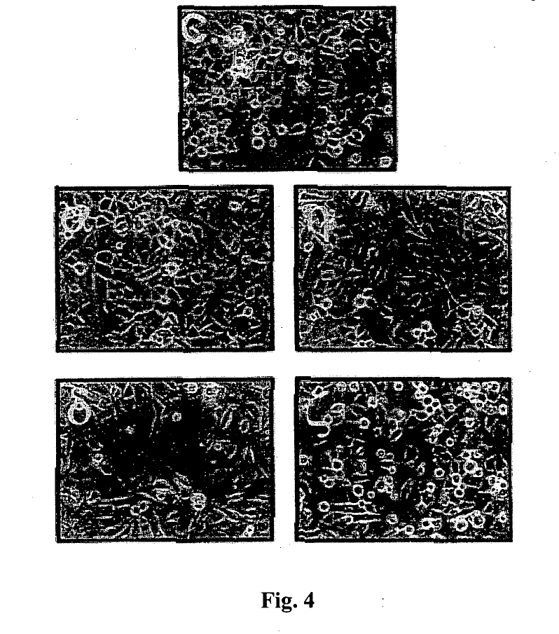

Each of the PKC adenovirus constructs employed induced a specific morphological change in primary keratinocytes (FIG. 4). Uninfected primary mouse keratinocyte cultures and β-galactosidase infected cells presented a cubidal morphology typical to the proliferative basal cell characteristics in culture. Regardless of isoform specificity all PKC over-expressing keratinocytes showed morphological changes typical to PKC activation including cell elongation and the appearance of neuronal like projections. However, each one of the PKC isoforms had a characteristic effect on keratinocyte morphology. PKCα infection induced stratification of keratinocytes, with a typical flattened morphology. In contrast, PKCη appeared as condensed clones of cells, presenting morphological characteristics of basal cells proliferating at prompt rate (FIG. 4). Two of the isoforms appeared to effect cell matrix as well as cell-cell associations. 18-48 hours following PKCδ infection, cells appeared elongated and extended with neuronal like projections. This was followed by gradual cell loss off the culture dish which occurred progressively in the course of the culture period. Over-expressing PKCζ keratinocytes appeared as rounded keratinocyte clusters, which were attached loosely to the culture dish and were gradually lost several days following infection.

### Example 5

### Distinct Localization of Over-Expressed PKC Isoforms in Infected Primary Keratinocytes

The distinct morphological changes were associated with distinct cellular localization as characterized by immunofluorescence analysis. In proliferating keratinocytes, PKCα, PKCδ and PKCζ were expressed in the cytoplasm as well as in the plasma membrane. Similarly to endogenous protein expression, PKCη isoform was localized to the keratinocytes' perinuclear region (FIG. 5). A dynamic change in distribution was associated with PKCδ and PKCζ, where succeeding cell detachment PKC isoform expression was predominantly localized to the cell membrane (FIG. 5).

### Example 6

### Regulation of α6β4 Expression by PKC Isoforms

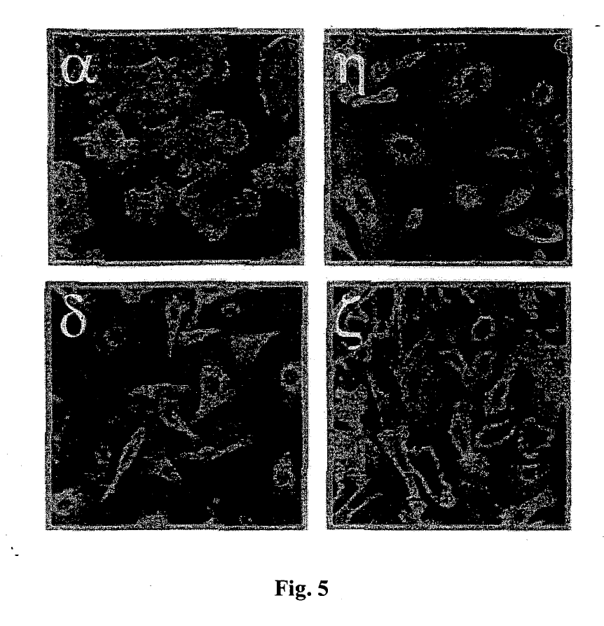

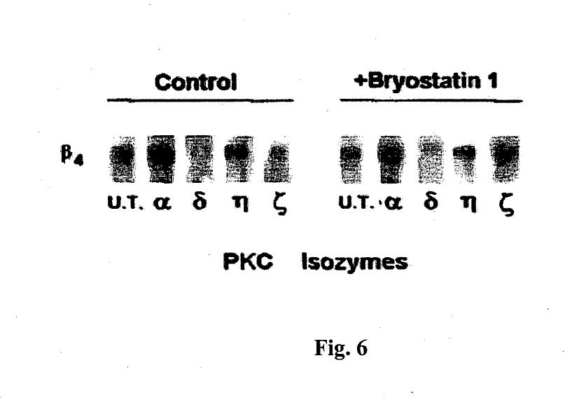

The ability of specific PKC isoforms to regulate proteins which are characteristic of the basal phenotype of the proliferative basal layer was examined. As down regulation of α6β4 integrin is one of the early events taking place during keratinocyte differentiation, the ability of the various PKC isoforms to regulate expression of the α6β4 integrin, an integrin which is specifically localized to the hemidesmosomes of the basal layer, was assessed. As can be seen in the immunoblot presented in FIG. 6, only PKCδ and PKCζ isoforms were able to down regulate α6β4 expression, in comparison to α6β4 integrin subunits levels in control keratinocytes. At the same time, α3 or β1 integrin subunits levels were not reduced. In contrast, consistently, over-expression of PKCα isoform resulted in increased α6β4 level two to three fold above control expression (FIG. 6). Over-expression of PKCη did not effect α6β4 protein expression. Several characteristics are associated with commitment of cells to differentiation and which follow the down regulation of the α6β4 protein including decrease in the proliferation rate, new keratin synthesis, cellular detachment and loss of attachment to basement membrane components. No changes in keratin expression were observed by over-expression of the different PKC isoforms. This included expression of K5 and K14, which are characteristic of the basal proliferating keratinocytes and K1 and K10, which are characteristic of the early stages of spinous differentiation. In addition, when proliferation rate was analyzed by ³H-thymidine incorporation there was no correlation between the loss of α6β4 expression and proliferation potential.

### Example 7

### Over-Expressed PKCη and PKCδ Induce Keratinocytes Proliferation in Vitro

Over-expression of PKCη and PKCδ significantly induced keratinocyte proliferation five and two fold above control levels, respectively (FIG. 7). PKCζ and PKCα did not affect cell proliferation.

### Example 8

### Over-Expressed PKC δ and ζ Induce Keratinocytes Detachment in Vitro

The adhesion properties of PKCδ and PKCζ over-expressing keratinocytes was studied. In comparison to control keratinocytes no change in adhesion potential to specific matrix proteins including laminin 1, laminin 5, fibronectin and collagen, was observed (data not presented). However, in cells over-expressing PKCδ and PKCζ isoforms, loss of cell contact with the culture dish was associated with gradual keratinocyte detachment from the culture dish (FIG. 4).

### Example 9

### PKC Isoforms Over-Expression Effects on Hemidesmosomal Localization of α6β4 Integrin

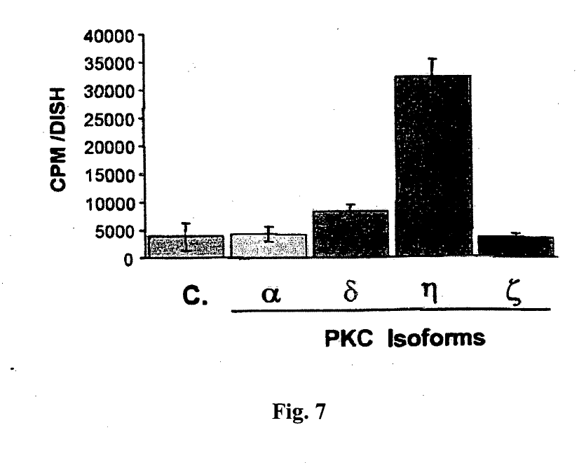

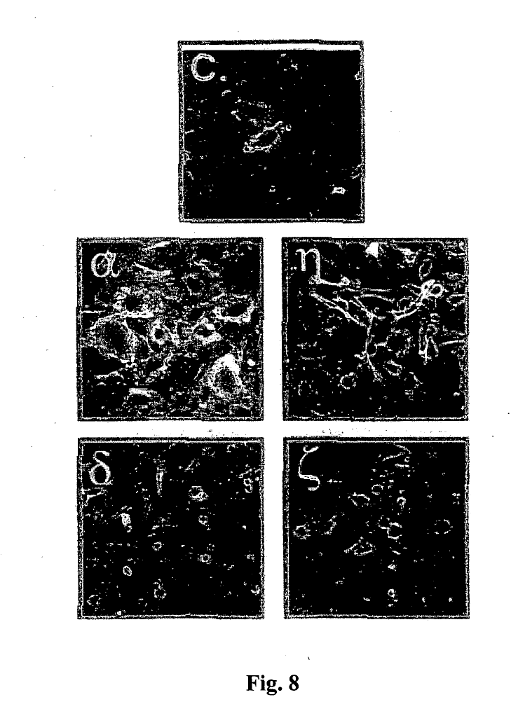

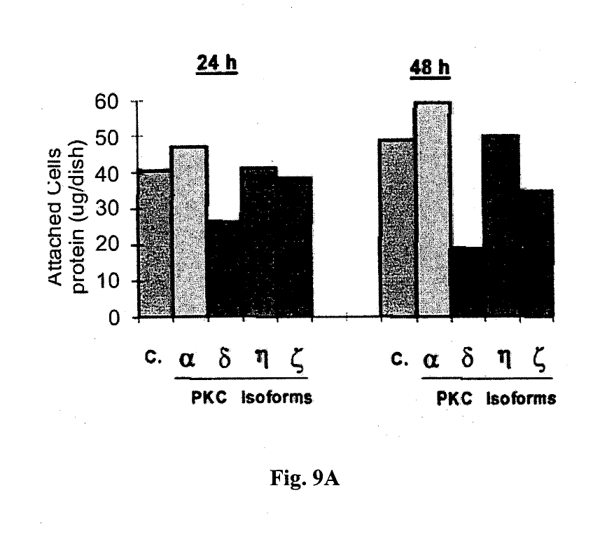

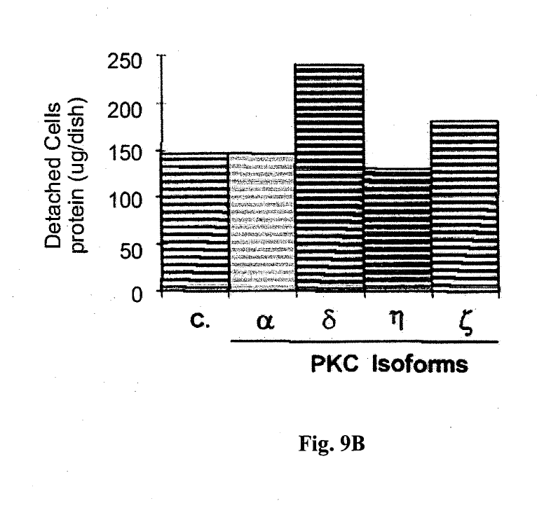

As α6β4 expression is essential for the formation of the hemidesmosomal adhesion complex, the association of α6β4 down regulation and cell detachment with α6β4 localization to the hemidesmosome was examined. FIG. 8 presents immunofluorescent analysis of α6β4 association with the hemidesmosomal complexes. As seen in FIG. 8, in comparison to control infected keratinocytes, up regulation of α6β4 integrin expression in over-expressing PKCα keratinocytes (FIG. 6) is associated with increased integration of α6β4 to the hemidesmosomal complexes. Cells over-expressing PKCη also induced association of α6β4 integrin with the hemidesmosomal complexes, although less than observed in over-expressing PKCα cells. As expected, the significant down regulation of α6β4 integrin in PKCδ and PKCζ over-expressing keratinocytes was found to be associated with decreased integration of α6β4 with the cells' hemidesmosomal complexes (FIG. 8). These results suggest that α6β4 integrin plays an important role in cell-matrix association and keratinocytes anchoring to the underlying basement membrane. Furthermore, PKCδ and PKCζ mediated α6β4 down regulation, initiate keratinocyte cell detachment in a pathway distinct from the keratinocyte differentiation processes. Finally, in order to link PKC mediated α6β4 down regulation, decrease hemidesmosomal α6β4 integration and specific morphological changes to keratinocyte detachment, the changes in the amount of attached and detached cells over-expressing the different PKC isoforms during the culture period were followed. In FIG. 9, attached cells were counted in cultures 24 and 48 hours following PKC adenoviral infection. As can be clearly observed, both PKCδ and PKCζ induced cell loss in vitro. In parallel, the loss of cells in culture was correlated with the increase in cells floating in the overlaying medium. These results indicate that PKCδ and PKCζ are important for control of the detachment step associated with the early stages of cell differentiation and migration.

### Example 10

### PKCη Differentially Regulates Keratinocyte Proliferation and Differentiation Under Physiological Settings

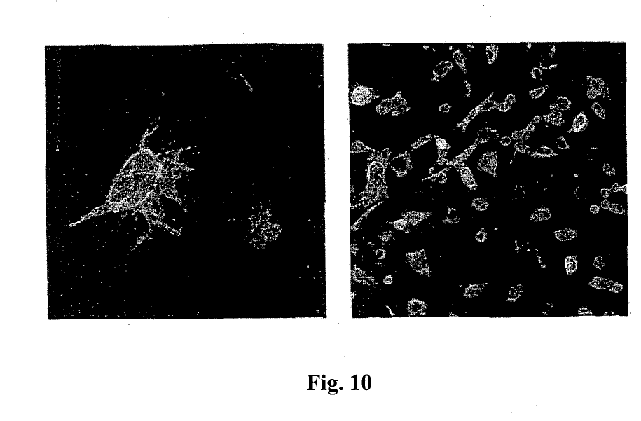

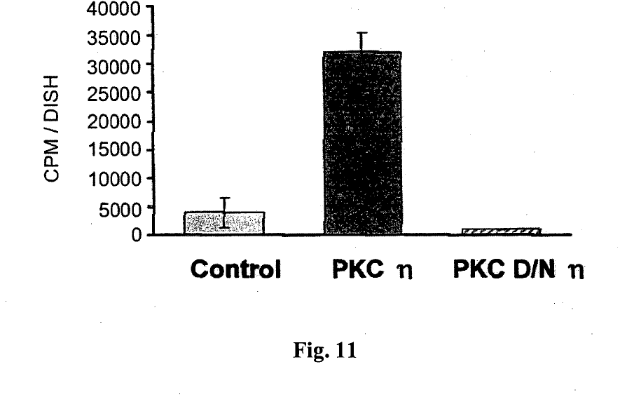

As clearly shown in FIG. 7, cells over-expressing PKCη isoform proliferate at an accelerated rate, five to seven times above control uninfected cells, and consistently higher then keratinocyte cultures over-expressing other PKC isoforms. However, the induction of proliferation was dependent on the differentiation state of the keratinocytes as determined by regulating the Ca²⁺ concentrations in the medium. In proliferating keratinocytes maintained under low Ca²⁺ concentrations (0.05 mM) endogenous PKCη was localized to the perinuclear region of majority of the proliferating cells (FIG. 10). Under these conditions, PKCη over-expression induced a dramatic increase in keratinocyte proliferation (FIG. 11). However, when keratinocytes were differentiated by elevating the Ca²⁺ concentrations to 0.12 mM, over-expression of PKCη did not induce proliferation but further stimulated keratinocyte differentiation. These results suggest that over-expressed PKCη induces proliferation only in physiologically proliferating cells but does not interfere with cellular differentiation. Divergence in regulation of PKCη expression was also seen in vivo. PKCη expression in actively proliferating skin as well as neuronal cells of the embryo was identified while in the mature adult brain no PKCη was observed and in the epidermis PKCη was localized to the granular layer in skin.

### Example 11

### PKCη and DNPKCη Over-Expression Specifically Regulates PKC Localization and Cellular Morphology

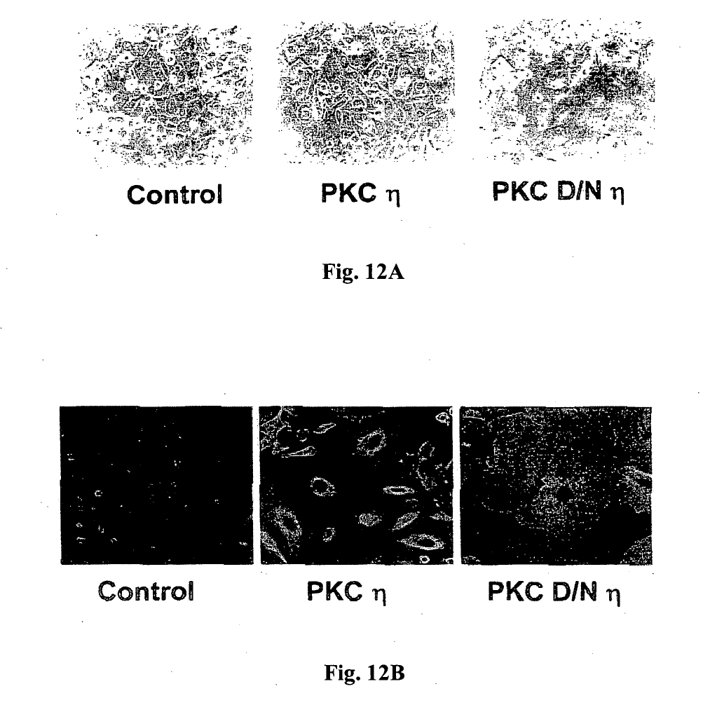

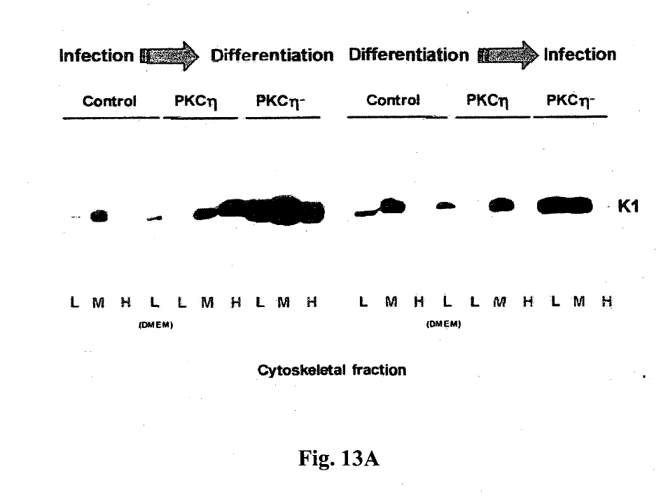

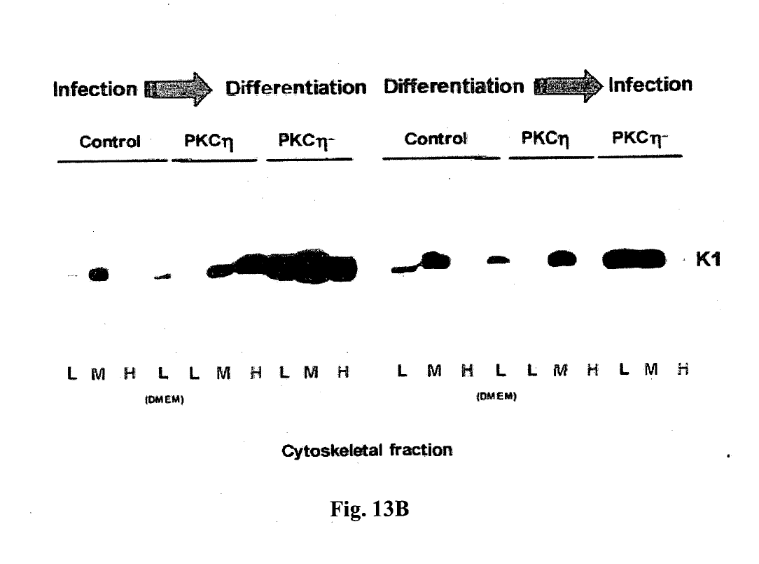

To further corroborate the results which support a positive role for PKCη in both states of proliferation or differentiation in keratinocytes, the effects of a kinase inactive dominant negative (DN) adenovirus PKCη construct were analyzed by studying the effect of infection in proliferating and differentiating keratinocytes. As seen in FIG. 12, adenoviral infection of both PKCη and DNPKCη were efficient in both the proliferation and differentiation states. As predicted, in proliferating keratinocytes DNPKCη induced keratinocyte differentiation with a dramatic change in cell morphology including flattening of the cells, loss of cell-cell boundaries similarly to the morphological changes associated with Ca²⁺ induced differentiation (FIGS. 12A-12B). Furthermore, these changes were associated with shut off of keratinocyte proliferation (FIG. 11) and a dramatic induction of differentiation markers including keratin 1, keratin 10, loricrin and filaggrin, which were elevated to similar levels presented in normal skin in vivo (FIGS. 13A-13B). At the same time, upon initiation of the differentiation program, over-expression of DNPKCη did not abrogate Ca²⁺ induced differentiation. These results suggest that PKCη and DNPKCη can be used for differentially regulating keratinocyte proliferation and differentiation under physiological settings.

### Example 12

### In Vivo Experiments—Effect of PKCη in Wound Healing

In order to test the ability of PKCη to differentially regulate cell proliferation and differentiation in vivo, the ability of PKCη to induce healing of full incisional wounds created on the back of nude mice was assessed. The ability of the keratinocytes to express the exogenous recombinant protein was verified by utilizing a control β-gal adenovirus. As can be seen in FIG. 14, two weeks after infection, β-gal expression is maintained in vitro keratinocytes as well as in vivo skin. Interestingly, when the wound healing process was examined in mice after local infection with control, PKCα and PKCη adenovirus constructs, only PKCη induced the formation of granulation tissue as early as four days following topical infection. This included also the organized formation of muscle, fat and dermal layers. At the same time, in control and PKCα infected skins, condensed granulation tissue was not noticed and no closure of the wound was observed (FIG. 14). Therefore, PKCη can be considered as a primary candidate in regulating proliferation and differentiation of skin in the induction of wound healing processes.

### Example 13

### Insulin Specifically Induces Translocation of PKCδ in Proliferating Keratinocytes

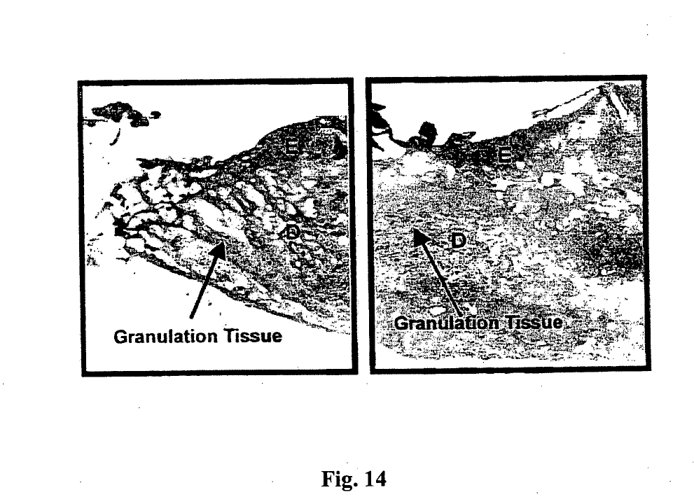

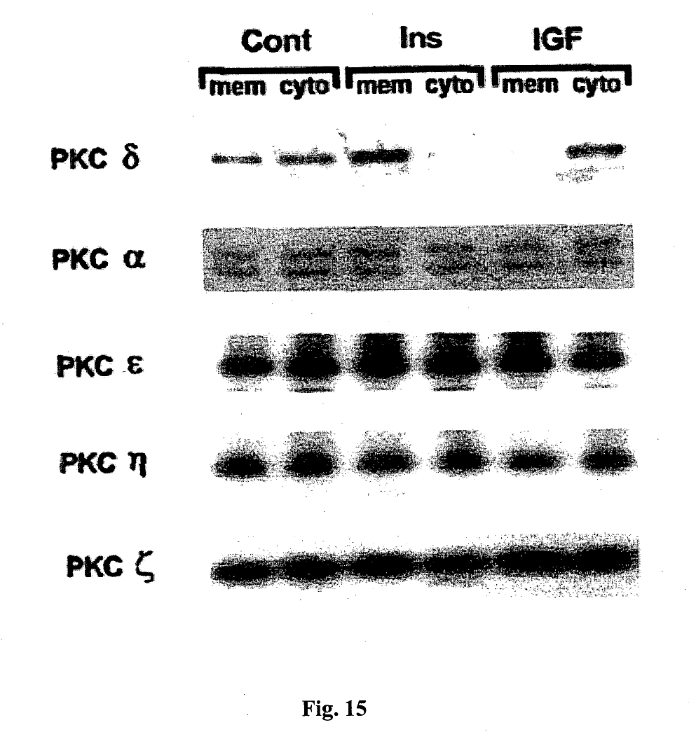

Two PKC isoforms expressed in skin were found to affect keratinocyte proliferation: PKCη and PKCδ. In order to try and identify the endogenous factors, which activate specific PKC isoforms regulating skin proliferation, the ability of several growth factors which are known to promote keratinocyte proliferation including: EGF, KGF, insulin, PDGF and IGF1 to activate specific PKC isoforms in a growth dependent manner, was assessed. PKC isoforms α, δ, ε, η and ζ are expressed in the skin. As activation of PKC isoforms is associated with their translocation to membrane fractions, the effects of these growth factors on the translocation of the various PKC isoforms from cytosol to the membrane were examined. As seen in FIG. 15, as early as 5 minutes following stimulation, insulin specifically induced translocation of PKCδ from the cytoplasm to the membrane fractions. Membrane expression of PKCδ was maintained for several hours following insulin stimulation. In contrast, IGF1 reduced PKCδ expression in the membrane and increased its relative level of expression in the cytoplasm fraction. No other growth factor significantly affected PKCδ translocation and localization. No change in distribution of the other PKC isoforms was seen following stimulation by any of the growth factors including IGF 1 and insulin.

### Example 14

### Insulin Specifically Induces Activation of PKCδ in Proliferating Keratinocytes

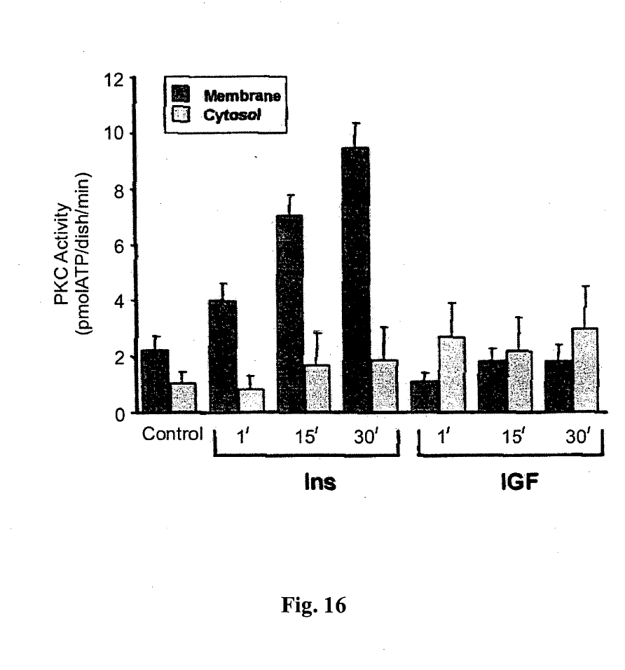

In order to determine whether the translocation of PKCδ is sufficient for activation, kinase activity of PKC immunoprecipitates from the cytoplasm and membrane fractions of insulin and IGF1 treated keratinocytes was measured. As shown in FIG. 16, insulin but not IGF1 increased activity of PKCδ in the membrane fraction. No elevation in PKCα activity was observed in the cytoplasm fraction. The insulin-induced activation was specific for PKCδ and no activation of PKCs α, ε, η or ζ was observed for up to 30 minutes following insulin stimulation. Altogether, these results suggest selective stimulation by insulin, but not by IGF1, of PKCδ activation.

### Example 15

### Insulin and IGF1 Have an Additive Effect on Keratinocyte Proliferation

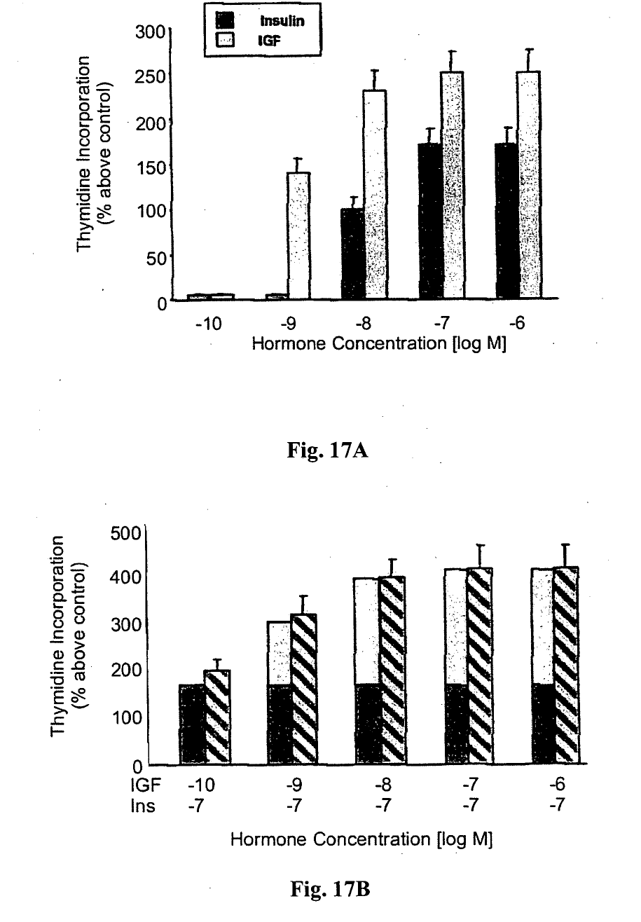

In order to analyze if the specific activation of PKCδ signifies specific insulin-induced mitogenic pathway in keratinocytes, the mitogenic effects of both insulin and IGF1 were examined by studying their ability to induce keratinocyte proliferation as measured by thymidine incorporation. As shown in FIG. 17A, both insulin and IGF1 stimulated thymidine incorporation in a dose dependent manner with maximal induction achieved at 10⁻⁷ and 10⁻⁸ M, respectively. At each concentration, the maximal stimulation by IGF1 was greater than that by insulin. Interestingly, at all concentrations, when both hormones were given together, the mitogenic effects were additive (FIG. 17B). These results suggest that insulin regulates keratinocyte proliferation through a distinct pathway independent of IGF 1 induced keratinocyte proliferation.

### Example 16

### The Association Between Insulin-Induced PKCδ Activation and Insulin-Induced Keratinocyte Proliferation

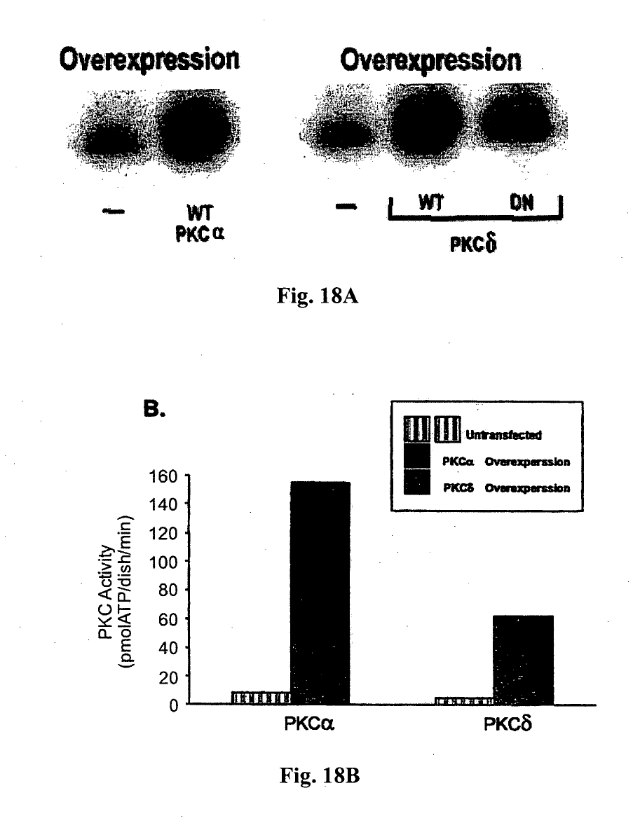

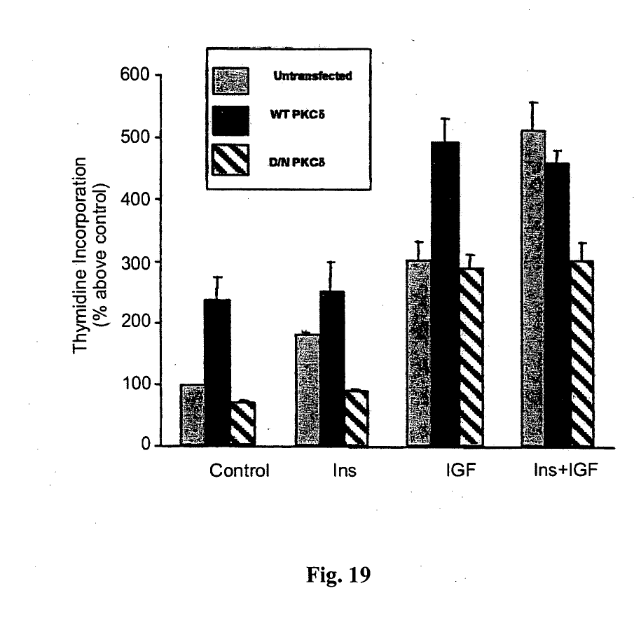

In order to directly study the association between insulin-induced PKCδ activation and insulin-induced keratinocyte proliferation, recombinant PKC adenovirus constructs were used to over-express both wild type PKCδ (WTPKCδ) as well as a kinase-inactive dominant negative mutant of PKC, which abrogates the endogenous PKCδ activity (DNPKCδ). The effects of over-expression of WTPKCδ and DNPKCδ on insulin-induced keratinocyte proliferation were examined. Both constructs, as well as a PKCα construct, were efficiently expressed in keratinocytes (FIG. 18A). Furthermore, infection with PKCδ and PKCα induced isoform-specific PKC activity several fold above control levels (FIG. 18B). As expected, over-expression of DNPKCδ did not induce PKC activity. As can be seen in FIG. 19A, insulin treatment of untransfected cells or over-expression of WTPKCδ without insulin treatment, increased thymidine incorporation to approximately identical levels, two to three fold over untreated cells, or cells transduced with PKCα. Moreover, addition of insulin to cells already over-expressing WTPKCδ did not cause any additional increase in thymidine incorporation. IGF1 increased thymidine uptake similarly in both non-infected cells and in cells over-expressing WTPKCδ and PKCα (FIG. 19A). The direct involvement of PKCδ in insulin induced proliferation was further proven by abrogating PKCδ activity. As seen in FIG. 19B, basal thymidine incorporation in cells over-expressing the dominant negative PKCδ was slightly, but significantly, lower than that in non-infected cells. Over-expression of DNPKCδ completely eliminated insulin-induced proliferation but did not affect IGF1-induced proliferation. Moreover, the additive effects of insulin and IGF1 was reduced to that of IGF1 alone.

### Example 17

### Specificity of PKCδ Activation to the Insulin-Mediated Pathway

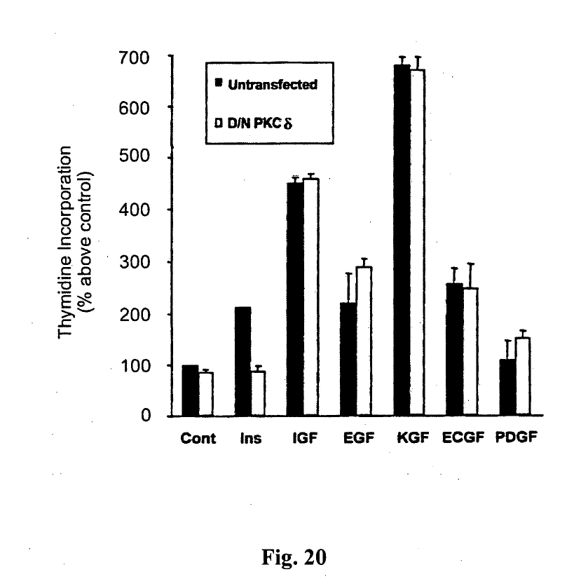

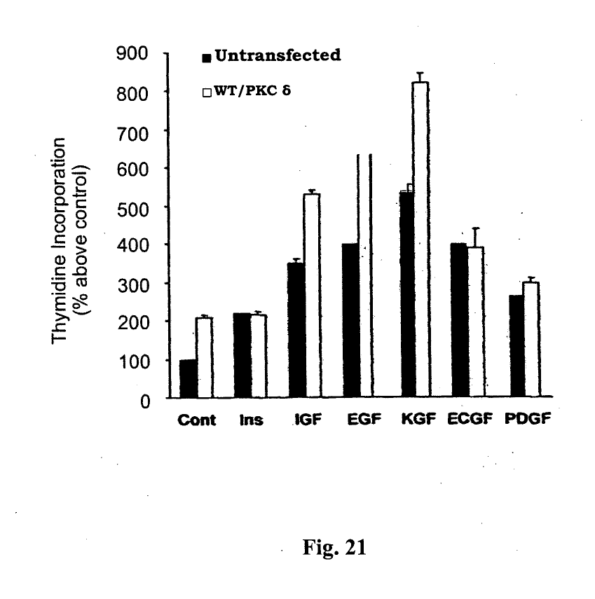

The specificity of PKCδ activation to the insulin-mediated pathway was analyzed by investigating the effects of PKCδ and DNPKCδ on the mitogenic response to a variety of growth factors including: IGF1, EGF, KGF, ECGF and PDGF. As seen in FIG. 20, the over-expression of DNPKCδ selectively eliminated the proliferative effects induced by insulin but did not block those of any of the other growth factors tested. However, the over-expression of PKCδ mimicked insulin induced proliferation and did not affect IGF1 induced proliferation. The proliferation induced by stimulation with EGF and KGF was increased (FIG. 21). These data indicate that PKCδ activation by insulin, mediates proliferation of keratinocytes through a pathway involving PKCδ and that this pathway is upstream of EGF and KGF signaling, two major growth factors known to regulate keratinocyte proliferation. Overall, insulin was found to be a specific regulator of PKCδ activity, which could be a specific candidate in regulating keratinocyte proliferation induced by insulin, EGF and KGF.

### Example 18

### Insulin-Induced PKCδ Activity and Keratinocyte Proliferation is Mediated by STAT3 Transcriptional Activation

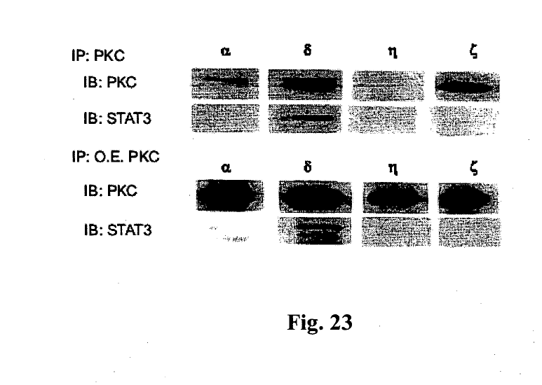

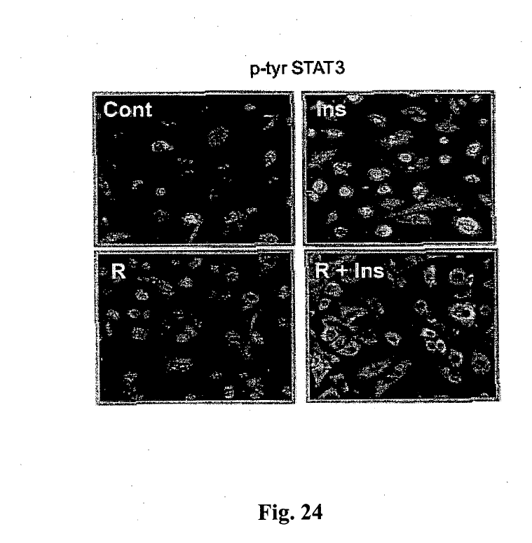

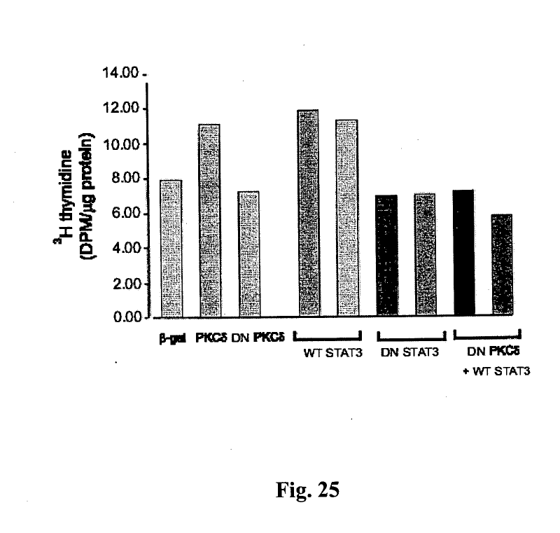

The role of PKCδ in insulin signaling was further characterized and found to involve induction of transcriptional activation mediated by STAT3. As seen in FIG. 23, in primary keratinocytes, PKCδ was shown to specifically associate with STAT3. Following insulin stimulation, PKCδ is activated and in turn phosphorylates and activates STAT3 (FIG. 24). Moreover, abrogating PKCδ activity by a pharmacological inhibitor (rottlerin) inhibits activation as well as nuclear translocation of STAT3. Furthermore, as seen in FIG. 25, overexpression of STAT3 induces a similar proliferation as that induced by insulin and by overexpression of PKCδ and abrogation of PKCδ activity by overexpression of a dominant negative PKCδ mutant abolishes the ability of STAT3 to induce keratinocyte proliferation. Overall these results suggest that insulin and PKCδ play a role in transcriptional activation associated with keratinoycte proliferation.

### Example 19

### PKCδ and PKCζ are Essential to the Wound Healing Process In Vivo

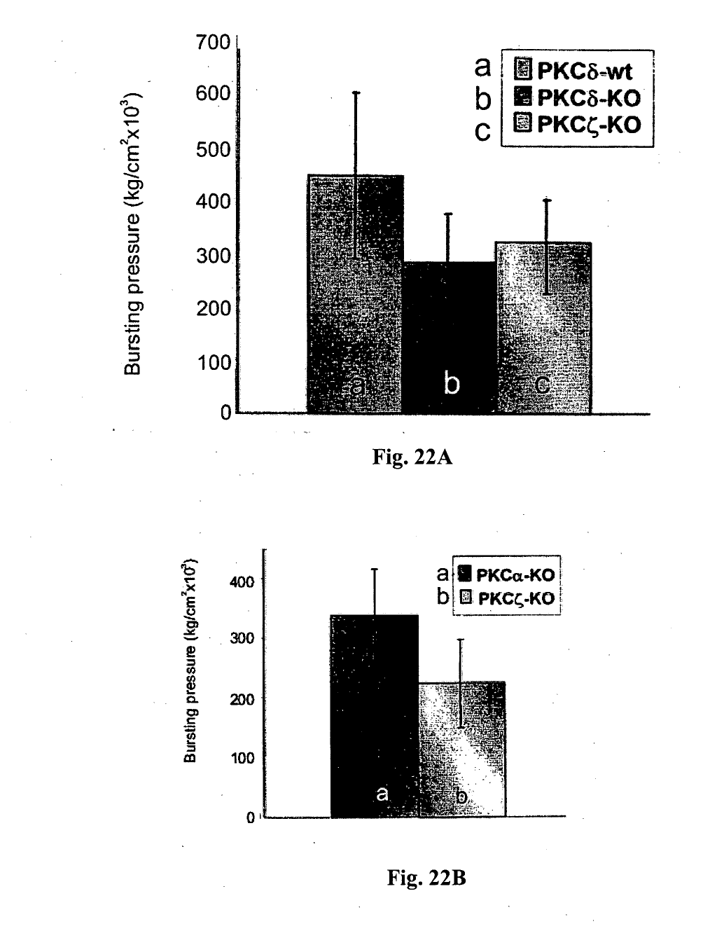

The importance of PKC isoforms in the wound healing process in vivo was established utilizing isoform specific PKC null mice. As seen in FIGS. 22A-22B, when full thickness wounds were created on the back of PKCδ, PKCζ, PKCα null mice (knock-out, KO) and their wild type littermates, delayed wound healing was observed in PKCδ and PKCζ but not PKCα null mice. This data indicates that even in the absence of diabetic background, specific PKC isoforms are essential for the wound healing process in skin.

### Example 20

### Single vs. Multiple Applications of Insulin for Wound Healing In Vivo

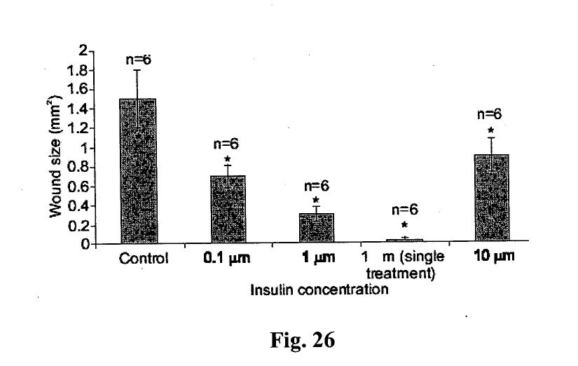

Wounds were effected on the back of 8-10 week old C57BL mice by incision and were treated as follows: (i) insulin 0.1 μM applied daily for 7 days; (ii) insulin 1 μM applied daily for 7 days (iii) insulin 10 μM applied daily for 7 days; (iv) insulin 1 μM applied once 4 days after wounding; and (v) vehicle (PBS) control applied daily for 7 days. All mice were sacrificed seven days after wounding and their open wound areas were measured. As seen in FIG. 26, a daily treatment of insulin at 1 μM concentration was significantly more effective than daily treatments of insulin at a lower (0.1 μM) or a higher (10 μM) concentration. Surprisingly, the treatment of a single application of insulin at 1 μM concentration was substantially more effective than the treatment of seven repeat daily applications of insulin at the same concentration.

Since the observed wounds were covered with a scar tissue it was difficult to correctly assess the actual closure of the wound and the formation of reconstructed epidermis. Therefore the effects of insulin on epidermal and dermal closure of wounds tissue were determined by histological parameters. Epidermal closure of wounds was determined by staining wound sections with Keratin 14 antibody (K14, Babco-Convance, Richmond, Calif., USA) which highlighted the formation of basal cells at the wound gap. Dermal closure of wounds was considered positive if at both wound sides the dermis could be observed in a single field observed under a light microscope at ×100 magnification.

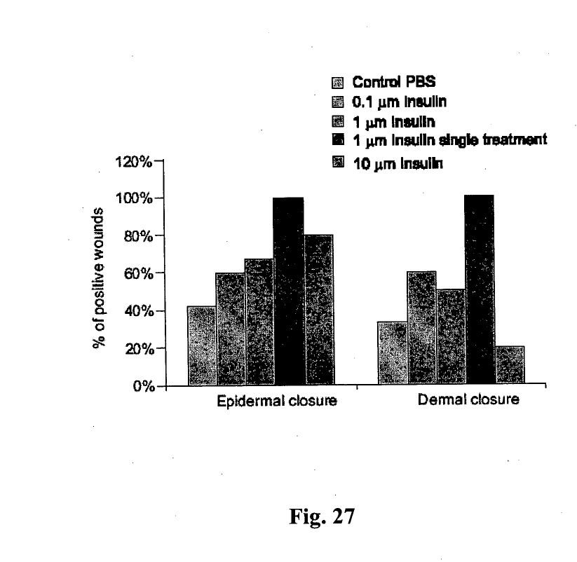

As seen in FIG. 27, all insulin treatments effectively promoted epidermal and dermal closure. Similarly to the results shown in FIG. 26, a daily treatment of insulin at 1 μM concentration was significantly more effective than a daily treatment of insulin at 0.1 μM, or 10 μM concentrations. In addition, a single application of insulin at 1 μM concentration was substantially more effective than of seven repeat daily applications of insulin at the same concentration.

Hence, these results clearly substantiate the therapeutic efficacy of insulin on wound healing in vivo as determined by morphological as well as histological parameters. The results surprisingly show that determining the optimal number and/or frequency of applications of insulin is a critical step for treating wounds properly.

### Example 21

### Combining Insulin and Platelet-Derived Growth Factor (PDGF-BB) for Wound Healing In Vivo

Wounds were perfomed on the back of 8-10 week old C57BL mice by incision and were treated with a single application 4 days after wounding as follows: (i) vehicle (PBS) control; (ii) insulin 1 μM (iii) PDGF-BB 10 μM (R&D Systems, Minneapolis, USA); and (iv) insulin 1 μM+PDGF-BB 10 μM. Seven days post wounding, all mice were sacrificed and wounds were histologically analyzed for epidermal and dermal closure such as described in Example 20 above.

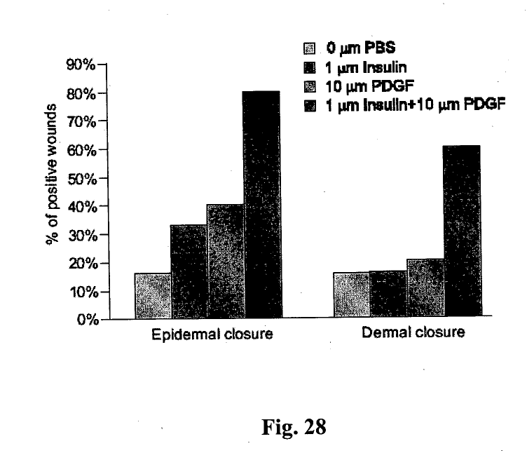

As seen in FIG. 28, a treatment with either insulin or PDGF-BB alone was partially effective on epidermal closure (30% and 40%, respectively) and on dermal closure (10% and 20%, respectively). However, the treatment of insulin and PDGF-BB combined exhibited a synergistic effect on epidermal closure (ca. 80%) as well as dermal closure (ca. 60%). Thus, the results show that combination of insulin and PDGF-BB affect wound healing in a synergistic manner. The results further indicate the potential of combining insulin with other growth factors or transforming factor such as EGF, TGFβ, KGF for therapeutic treatment of wounds.

### Example 22

### Combining Insulin and PKCα Inhibitor for Wound Healing In Vivo

Wounds were performed on the back of 8-10 week old C57BL mice by incision and were treated daily for 7 days with either vehicle (PBS) control or with 0.67 μM insulin (herein referred to as HO/01; Humulin, Eli Lilly, USA) combined with the PKCα inhibitor (PKCα pseudosubstrate N-myristoylated peptide of SEQ ID NO:1, herein referred to as HO/02; CalbioChem, San Diego, Calif., USA). Seven days after wounding, all mice were sacrificed and treated wounds were analyzed for wound closure, epidermal closure, dermal closure, and spatial differentiation of epidermal cells. Wound closure was determined by measuring the open wound area. Dermal closure of wounds was considered positive if at both wound sides the dermis could be observed in a single field observed under a light microscope at ×100 magnification. Epidermal closure of wounds was determined by staining wound sections with K14 antibody, which highlighted the formation of basal cells at the wound gap. Spatial differentiation of epidermal cells was determined by staining wound sections with K1 antibody, which highlighted newly formed epidermal cells.

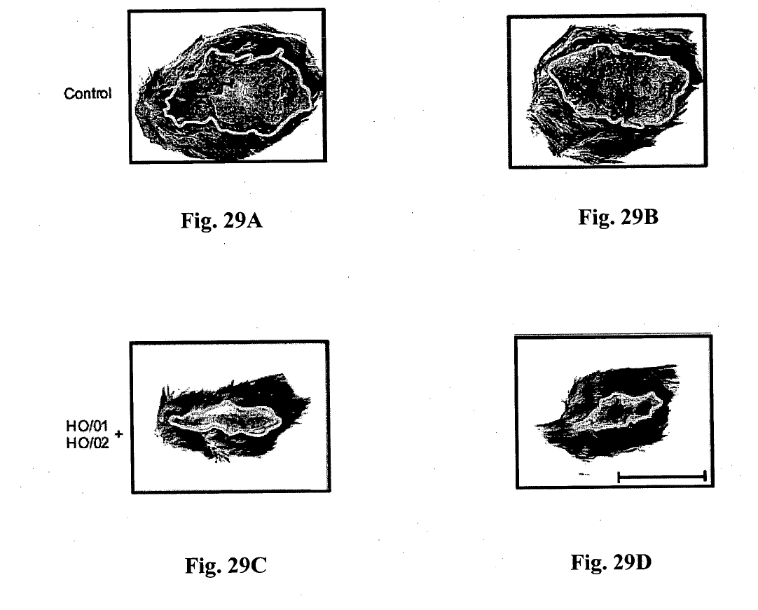

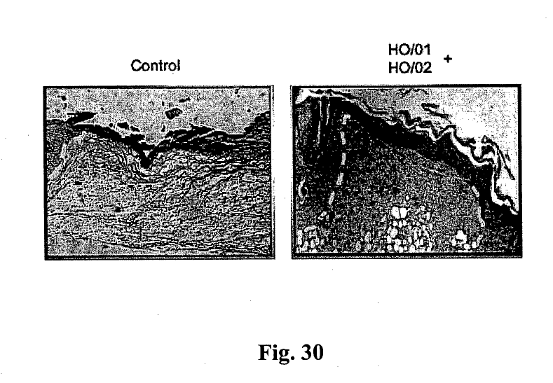

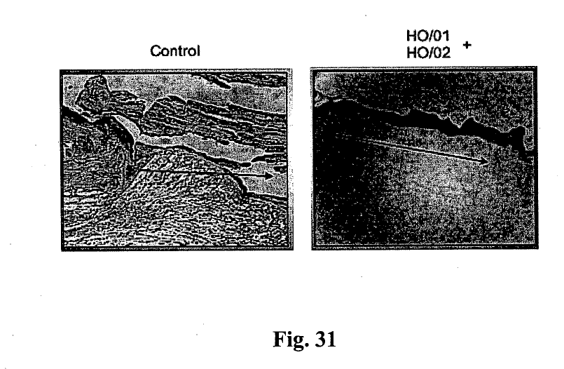

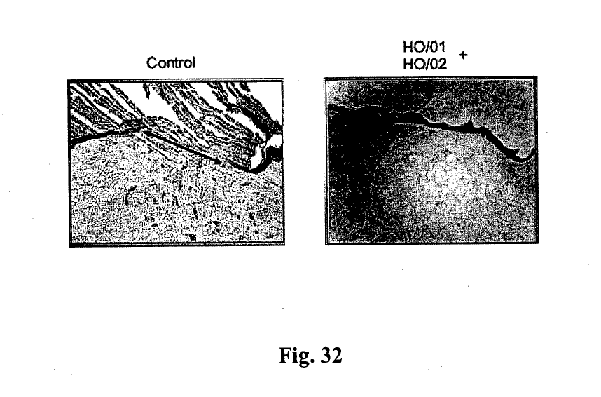

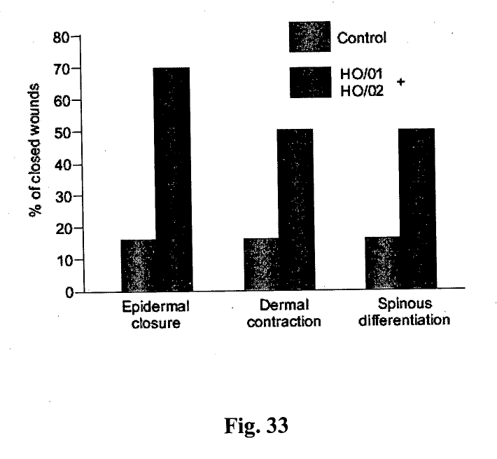

As illustrated in FIGS. 28-32, the combined application of insulin and (HO/01) and the PKCα inhibitor (HO/02) substantially promoted wound closure (FIGS. 29A-29B), dermal closure (FIG. 30), epidermal closure (FIG. 31), and spatial differentiation of epidermal cells (FIG. 32). As can be seen in FIG. 33, the treatment of insulin HO/01 combined with PKCα: inhibitor HO/02 increased wounds epidermal closure from ca. 15 to 70%, increased dermal closure from ca. 15 to 50% and increased spatial differentiation of epidermal cells from ca. 15 to 50%, as compared with the vehicle control, respectively.

Hence, the results show that a therapeutic treatment of wounds by insulin combined with a PKCα inhibitor effectively promotes epidermal closure, dermal closure, spatial differentiation of epidermal cells, and subsequently wound healing.

### Example 23

### Combining Insulin and PKCα Inhibitor Circumvent Adverse Side Effects Caused by Insulin Only Treatment

Wounds were performed on the back of 8-10 week old C57BL mice by incision and were treated daily for 7 days with either vehicle (PBS) control or with 1 μM insulin (Humulin, Eli Lilly, USA) or a mixture of 1 μM insulin combined with 1 μM of the PKCα inhibitor of SEQ ID NO: 1 (HO/02). Seven days after wounding all mice were sacrificed and treated wounds were histologically analyzed for proliferative capacity of the epidermis (proliferating cell nuclear antigen-PCNA), angiogenesis, inflammation, epidermal cells and the remodeling processes at the wound gap.

As shown in Table 1, the insulin-only treatment caused a substantial increase in the incidence of abnormal angiogenesis in the wound area, as compared with the PBS control (60% and 25%, respectively). Since the wound healing process involves rapidly proliferating epidermal cells, such increased angiogenesis may also increase the risk of impaired wound closure by delaying formation of normal granulation tissue. On the other hand, when insulin was combined with PKCA inhibitor HO/02 no abnormal angiogenesis was observed in the treated wound area.

TABLE 1
The effect of insulin only and insulin combined with PKCα inhibitor
on the severity of angiogenesis at the wound area

| | Proliferative capacity of | |
|---|---|---|
| | the epidermis (layers of | |
| Treatment | basal cells, PCNA) | Angiogenesis |
| PBS (control) | 5 | high abnormal |
| N = 4 | 7 | normal |
| | 8 | normal |
| | 6 | normal |
| | average 6.5 | |
| Insulin only | 8 | high abnormal |
| N = 5 | 8 | normal |
| | 6 | normal |
| | 5 | high abnormal |
| | 5 | high abnormal |
| | average 6.4 | |
| Insulin + PKCα | 6 | normal |
| inhibitor | 6 | normal |
| N = 5 | 4 | normal |
| | 2 | normal |
| | 3 | normal |
| | average 4.2 | |

In addition, the insulin-only treatment resulted in increased inflammation, hyperplasia of epidermal cells, delayed differentiation of the spinous layer of epidermal cells and increased scarring. None of the adverse side effects, which resulted from the insulin-only treatment, were observed when the PKCα inhibitor was combined with insulin.

### Example 24

### PKCα Inhibitor Reduces Wounds Inflammation

Late and severe inflammatory response in wounds may suppress the process of healing, thus preventing such inflammation from development may promote the wound healing process. Accordingly, the effect of PKCα inhibitor HO/02 and insulin on wound inflammation was tested in the following experiment.

Wounds were performed on the back of C57BL mice by incision and were treated daily for 7 days with: (i) PBS, control; (ii) 1 μM of the PKCα inhibitor of SEQ ID NO: 1; (iii) 1 μM insulin (Humulin, Eli Lilly, USA); or (iv) a mixture of 1 μM PKCα inhibitor and 1 μM insulin. Seven days after wounding all mice were sacrificed and the treated wounds were observed for inflammation under a microscope. The resulting incidences of severe inflammation observed in the wound area are summarized in Table 2.

As shown in Table 2, administering the PKCα inhibitor to wounds caused a substantial (33.3%) decrease of severe wound inflammation incidence, as compared to control. Insulin alone had no anti-inflammatory effect under the experimental conditions.

TABLE 2

| | Incidence of severe |
|---|---|
| Treatment | inflammation in wound (%) |
| PBS control | 60 |
| PKCα inhibitor | 40 |
| Insulin | 56 |
| PKCα inhibitor + insulin | 50 |

These results indicate that a PKCα inhibitor can be used in therapy to control severe inflammation of wounds. The demonstrated capacity of the PKCα inhibitor of SEQ ID NO:1 to reduce inflammation, coupled with its capacity to promote epidermal closure, dermal closure and spatial differentiation of epidermal cells (see Example 22 hereinabove), makes it a potentially most effective therapeutic agent for wound healing.

### Example 25

### Efficacy of Insulin in Combination with the PKCα Inhibitor HO/02 in Wound Healing in STZ-Induced Diabetic Mice

Full thickness skin incisions (20 mm) were performed on the upper back of anesthetized streptozocin (STZ)-injected (175 mg/kg body weight) diabetic, citrate buffer injected (6 mice/group) non-diabetic, and non-injected (6 mice/group), C57BL/6J mice. Periodical blood glucose monitoring was performed by measuring glucose levels from tail vein after STZ injection. Only mice exhibiting blood glucose levels higher than 450 mg/dl were taken into the study. Following incision, STZ-induced diabetic wounds were treated daily by application of PBS (7/6 mice/group), insulin (0.1 units/ml) (6 mice/group), the PKCα inhibitor of SEQ ID NO:1 (herein HO/02) (1 μg/ml) (7/5 mice/group) or a formulation (herein designated HO/03/03) containing insulin and the N-myristoylated PKCα inhibitor of SEQ ID NO:1 (7/6 mice/group) directly to the surgical dressing. Biopsies were collected 9 days post-wounding. Wounds were excised from euthanized animals for evaluation of wound-healing parameters by histology and immunohistochemistry.

Wounds were assessed for epidermal closure by Keratin 14 staining. Wounds were considered closed when complete epidermal staining was observed across the wound gap. Dermal contraction was considered when both dermal edges were visible in a fixed field ×100 magnification utilizing H&E staining. Epidermal differentiation was assessed by Keratin 1 staining. Wounds that displayed positive staining across the entire wound gap were considered as differentiated (K1 positive).

As shown in Table 3, STZ-induced diabetic animals were wound-healing impaired. Comparison of important wound healing parameters in diabetic treated, untreated and non-diabetic mice revealed that the group treated with the formulation HO/03/03 exhibited a synergistic healing effect. The synergy was evident in all critical healing stages including epidermal closure (71% vs. 17% in the diabetic control p<0.05), epidermal differentiation (28% vs. 0%) and dermal contraction (33% vs. 0%).

The organization of hypodermis was assessed by the presence (or absence) of hypodermis at both wound edges. Granulation tissue formation was assessed by the presence of fibroblasts and collagen fibers in the wound bed. The wound was considered positive for granulation tissue formation when a continuous layer of granulation tissue was present in the wound gap. In addition, we have defined 3 specific histological parameters characterizing severe inflammation of diabetic wounds: (i) abscess formation at the wounded area, (ii) excessive leukocytosis (>100 cells in a fixed field (×200)) and (iii) high white blood cells (WBC)/red blood cells (RBC) ratio within the blood vessels shown in a fixed field (×200). When at least 2 of these parameters are present at the wound gap, the wound is considered severely inflamed.

TABLE 3
Histological analysis of wound healing parameters 9-days post wounding

| | Epidermal | Dermal | |
|---|---|---|---|
| | closure | closure | Epidermal |
| Treatment group | (K14) | (H&E) | differentiation (K1) |
| Diabetic + PBS | 17% | 0% | 0% |
| Diabetic + Insulin 0.1 units | 17% | 0% | 0% |
| Diabetic + HO/02 1 ug/ml | 17% | 17% | 17% |
| Diabetic + HO/03/03 | 71% | 28% | 33% |
| Non-diabetic | 100% | 17% | 100% |
| Buffer Citrate Injected + PBS | | | |

* Quantitative analyses of healing parameters were made as described above. Results are presented as percent of wounds in each group.

TABLE 4
Histological analysis of wound healing parameters
9-days post wounding

| | | Severe | Granulation |
|---|---|---|---|
| Treatment group | Hypodermis | inflammation | tissue |
| Diabetic + PBS | 25% | 67% | 42% |
| Diabetic + Insulin 0.1 units | 10% | 100% | 40% |
| Diabetic + HO/02 1 ug/ml | 10% | 30% | 50% |
| Diabetic + HO/03/03 | 43% | 28% | 86% |
| Non-diabetic | 100% | 17% | 92% |
| Buffer Citrate Injected + PBS | | | |

* Quantitative analyses of healing parameters were made as described above. Results are presented as percent of wounds in each group.

As shown in Table 4, diabetic animals exhibited impairment in other wound healing parameters such as the organization of hypodermis at wound edges, inflammation and granulation tissue formation. Diabetic associated impaired healing parameters were corrected by treatment with the formulation HO/03/03 as demonstrated by organization of the hypodermis at the wound gap edges (43% vs. 25%) and granulation tissue formation (86% vs. 42%). Treatment with HO/03/03 reduced inflammatory response in the wound gap (28% vs. 67%). Healing efficacy of the formulation agents alone (insulin or HO/02) exhibited only partial healing effects.

The results summarized above demonstrate that the formulation HO/03/03 exhibits a synergistic effect in overcoming diabetes related healing impairments in multiple healing parameters.

### Example 26

### Efficacy of Insulin in Combination with the PKCα Inhibitor HO/02 In Wound Healing In Vivo in the Pig Skin Model

Several wound healing studies were conducted in the pig model system for further understanding of the wound healing process as well as the healing effects of the formulation HO/03/03.

Full thickness 35-40 mm skin incisions were performed on the back of an anesthetized female pig (5 months old, 60-70 kg). Ten symmetrical incisions were performed on both sides of the back area at the same distance from the back bone (20 wounds total). The wounds were treated daily, twice a day, by application of 1 ml PBS (10 per group) or the formulation (herein designated HO/03/03) containing insulin 0.1 units and the N-myristoylated PKCα inhibitor 1 μg/ml of SEQ ID NO:1 (10 per group) directly to the wound area. Wounds were excised from sacrificed animals on 7 and 22 days post-wounding and morphological, histological and immunohistochemical assessments were performed.

As shown in Table 5, treatment with the formulation HO/03/03 accelerates wound healing by affecting epidermal migration and granulation tissue formation at the early stages of healing. Moreover, these wounds are large and prone to infections from environmental pathogens (animals are not kept in sterile conditions), yet HO/03/03 exhibits attenuation of the inflammatory response at the wound gap thus promoting healing progression.

TABLE 5
Histological analysis of efficacy of the formulation
HO/03/03 in pig 7-days post wounding

| | | Severe | Granulation tissue |
|---|---|---|---|
| Treatment group | Epidermal migration | inflammation | firmation |
| PBS | 30% | 60% | 50% |
| HO/03/03 | 80% | 30% | 85% |

*Results are presented as percent of wounds in each group

TABLE 6
Histological analysis of efficacy of the formulation HO/03/03
in pig 22-days post wounding

| Treatment | Epidermal | Epidermal | Dermal |
|---|---|---|---|
| group | closure | Differentiation | Contraction |
| PBS | 88% | 67% | 25% |
| HO/03/03 | 100% | 90% | 70% |

*Results are presented as percent of wounds in each group

When testing advanced healing stages, HO/03/03-treated wounds further exhibit accelerated healing. As shown in Table 6, no difference was noted in the epidermal closure, though dermal contraction and epidermis differentiation was significantly higher in treated wounds. It is important to emphasize that these wounds were performed on the same animal thus contributing to the marked healing effects of the treatment.

From the results summarized above it is readily understood that the formulation HO/03/03 promotes accelerated healing in the pig skin model affecting healing parameters in early as well as late wound healing stages.

### Example 27

### Efficacy of the Combination of the PKCα Inhibitor with PKCδ RACK in Wound Healing in Mice

As demonstrated in Examples 25-26, specific inhibition of PKCα through a specific pseudosubstrate inhibitor together with activation of PKCδ through insulin, exhibited a synergistic effect on wound healing parameters such as epidermal closure. In light of these results, we tested another combination in which PKCδ is activated by a different specific PKCδ activator, designated PKCδ RACK.

In vivo studies were conducted on C57BL mice that were subjected to wounding (5-6 mice/group). A full thickness 20 mm incision was performed on the upper backs of the animals. The wounds were treated topically daily either with PBS (control), PKCα inhibitor of SEQ ID NO:1 (HO/02) (1 μM), PKCδ RACK (25 μM) or the combination of the PKCα inhibitor (HO/02) and PKCδ RACK (1:1 per volume). Morphological and histological assessments were performed during the experiment time course. Paraffin-embedded sections from the widest part of the wound were immunohistochemically stained for epidermal cell marker Keratin 14 (K 14) for the indication of the new epidermis over the wound gap. Only wounds that exhibited full distribution of the marker over the wound gap were scored as positive. The results in Table 7 represent the percent of positive wounds per group.

As shown in Table 7, PKCδ activation by PKCδ RACK together with PKCαinhibition resembles results obtained in experiments where PKCα inhibition was coupled with PKCδ activation by insulin. Wound healing parameters such as epidermal closure were shown to be synergistically effected by the mentioned combinations. Thus, it may be concluded that specific PKCδ activation by modifying agents other than insulin might also serve as potential formulation components in promoting wound healing.

TABLE 7
Wound healing efficacy of the combination of PKCα inhibitor
(HO/02) with PKCδ RACK

| Treatment | Epidermal Closure |
|---|---|
| PBS control | 0% |
| PKCα inhibitor (HO/02) | 40% |
| PKCδ RACK | 20% |
| PKCα inhibitor + PKCδ RACK | 80% |

*Results are presented as percent of wounds in each group

### Example 28

### The Combined Effects of Modulating Expression and/or Activity of Specific PKC Isoforms in Skin Cells and Administering Various Agents to the Cells on Accelerating Wound Closure In Vitro

Materials and Methods

(i) Reagents.

Factor D (Adipsin) Human, PKCα Pseudosubstrate Inhibitor, PKCζ Pseudosubstrate Inhibitor, PKCη Pseudosubstrate Inhibitor, and Rosiglitazone, were obtained from CalbioChem (San Diego, Calif., USA); Recombinant TNFα Mouse was from R&D Systems (Minneapolis USA); GW 9662 was purchased from Cayman Chemical (USA); PDGF-BB, IL-6, KGF/FGF-7, IGF-1, and TGFβ2 were obtained from Cytolab (Israel); Epidermal Growth Factor (EGF), Mouse, was from Chemicon International (CA, USA); PKCδ RACK was from AnaSpec (CA, USA); Adiponectin was from MBL (Massachusetts, USA) and Copolymer-1 (Copaxone®) was obtained from Teva Pharmaceutical Industries Ltd. (Petach Tikva, Israel).

(ii) In Vitro Wound Closure Assay.

Keratinocytes and fibroblasts (dermal cells) were cultured for five days in Petri dishes (5 cm i.d.), then an artificial cross-over scratch was formed in each dish with a 200 μl pipette tip. The cultured cells were infected with adenovirus constructs capable of modulating expression and/or activity of specific PKC isoforms. Accordingly, wild-type (WT) PKC adenovirus constructs were used to activate specific PKCs, while dominant-negative (DN) PKC adenoviral constructs were used to inhibit specific PKCs. The cultured cells were further provided with one of the following agents: insulin (6.7×10⁻⁷ M), adiponectin (1 μg per dish), adipsin (2 μg/ml), IL-6 (1 μg per dish), GW9662 (1 μg per dish), KGF (1 μg per dish), TNFα (12 μg/ml), TGFβ, rosiglitazone, SRC inhibitors, PKCδ RACK (10⁻⁷M) and a PKCα pseudosubstrate inhibiting peptide (10⁻⁷M). The resulting wound closure levels were determined 24-48 hours following treatment using index values ranging from 0 (no closure) to 10 (complete closure).

Results: In experiments conducted on fibroblasts, primary skin fibroblasts were isolated and plated on non-coated Petri dishes as described above until confluence in low Ca²⁺ medium. Cells were either infected with various PKC expressing adenoviral constructs or treated with selected PKC modulating agents. PKCα, PKCβ, and PKCζ inhibition includes PKCα, PKCβ, or PKCζ dominant negative construct over-expression, respectively, or treatment with PKCα, PKCβ, and PKCζ pseudosubstrate inhibiting peptide (corresponding to the specific pseudosubstrate region of PKCα, PKCβ, and PKCζ, respectively). The infection was performed as follows: cells were infected with a 1:100 titer of a PKC dominant negative adenoviral construct for 1 h at 37° C. (rotation every 15 min). Treatment with the specific PKC isoform inhibiting peptides was performed as follows: cells were treated with 1 μM PKCα pseudosubstrate peptide of SEQ ID NO:1, 1 μM PKCβ pseudosubstrate peptide, 1 μM PKCζ pseudosubstrate peptide, all treatments were performed for 15 min. Thereafter, cells were treated with selective PKC isoform modulating agents: adipsin (2 μg/ml), insulin (0.1 unit/ml), hIL-6 (0.2 μg/ml, TGFβ (100 nM), KGF (0.2 μg/ml), adiponectin (0.2 μg/ml), GW9662 (PPAR-γ antagonist, 0.2 μg/ml); rosiglitazone (PPAR-γ agonist, 100 nM) and Src inhibitors cocktail (100 nM). Photo documentation was performed as listed, on day 0 and 24 hr post treatment. Magnification ×100. The results are shown in Table 8.

Primary skin fibroblasts were isolated and plated on non-coated Petri dishes as described above until confluence in low Ca²⁺ medium. Cells were either infected with PKCα dominant negative adenoviral constructs or treated with the PKCα pseudosubstrate inhibiting peptide of SEQ ID NO:1. Then cells were either co-infected with adenoviral constructs or treated with PKC isoform inhibitors or activators as follows: PKCζ, PKCη or PKCε inhibition included PKCζ, PKCη or PKCε dominant negative construct over-expression 1:100 titer or treatment with 1 μM PKCζ, PKCη or PKCε pseudosubstrate inhibiting peptide; PKCζ and PKCη activation included wild type PKCζ or PKCη adenoviral construct over-expression 1:100 titer (10⁷-10⁹ pfu); PKCε activation included wild type PKCε adenoviral construct over-expression 1:100 titer (10⁷-10⁹ pfu) or with specific activating agent such as PKCε RACK 100 nM; and PKCδ activation included wild type PKCδ adenoviral construct over-expression 1:100 titer (10⁷-10⁹ pfu) or with specific PKCδ activating agent such as bryostatin 10 nM or PKCδ RACK 100 nM. For the infection, cells were infected with a 1:100 titer (10⁷-10⁹ pfu) of a PKC dominant negative adenoviral construct for 1 h at 37° C. (rotation every 15 min). The results are summarized in Table 9.

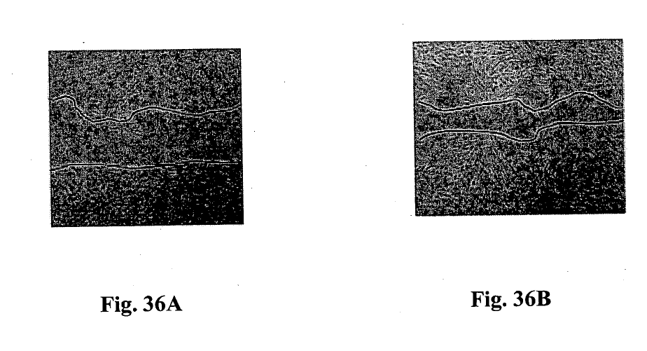

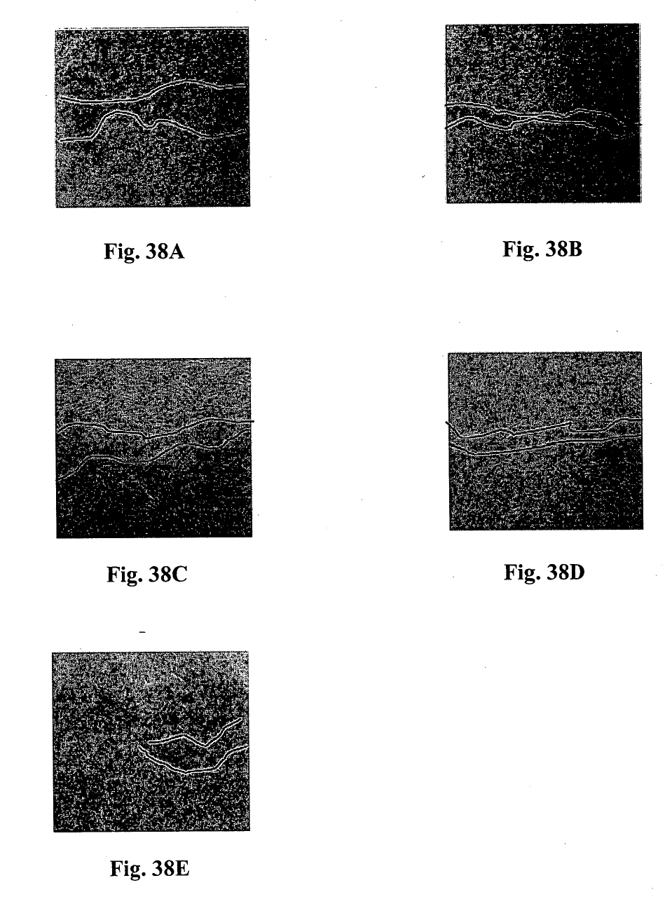

The results in Tables 8-9 of the treatment of fibroblasts in vitro model of wound closure by different combinations of modulation of two PKC isoforms show that inhibition of PKCα expression and/or activity in fibroblasts substantially promoted wound closure when combined with administration of adipsin or insulin to the cells (Table 8, wound closure index values of 10 and 8, respectively). Wound closure was also accelerated by the inhibition of PKCα combined with inhibition of PKCη, inhibition of PKCε, activation of PKCδ, or activation of PKCζ (Table 9, wound closure index values of 9, 9, 9 and 7, respectively; FIGS. 34A-34E). In addition, wound closure was promoted by inhibition of PKCζ combined with administration of KFG to the cells (Table 8, wound closure index value of 7; FIG. 36). Further in addition, wound closure was accelerated by the inhibition of PKCβ combined with administration of insulin, IL-6, KGF or GW9662 (Table 8, wound closure index values of 8, 7, 9 and 8, respectively; FIGS. 38A-38E).

TABLE 8
The effect of treatment combinations on the closure
of fibroblasts in vitro wounds¹

| | PKCα | PKCβ | PKCζ |
|---|---|---|---|
| | inhibition | inhibition | inhibition |
| Control | 4 | 4 | 2 |
| Adipsin | 10 | ND | ND |
| Insulin | 8 | 8 | 0 |
| IL-6 | 3 | 7 | 2 |
| TGFβ | ND | 1 | ND |
| KGF | 2 | 9 | 7 |
| Adiponectin | 3 | 3 | 2 |
| Rosiglitazone | 5 | 3 | 2 |
| GW9662 | 2 | 8 | 1 |
| SRC inhibitors | 3 | 2 | 1 |

¹Scale of closure was 0 (no closure) to 10 (complete closure).
ND = not determined.

TABLE 9
The effect of treatment combinations on the closure of fibroblasts in
vitro wounds¹

| | PKCα inhibition |
|---|---|
| Control | 4 |
| PKCη activation | 5 |
| PKCη inhibition | 9 |
| PKCδ activation | 9 |
| PKCε activation | 3 |
| PKCε inhibition | 9 |
| PKCζ activation | 7 |
| PKCζ inhibition | 1 |

¹Scale of closure was 0 (no closure) to 10 (complete closure)

In experiments conducted on keratinocytes, primary skin keratinocytes were isolated and plated on non-coated Petri dishes until confluence in low Ca²⁺ medium. Cells were either infected with various PKC expressing adenoviral constructs or treated with selected PKC modulating agents as follows: PKCα or PKCζ inhibition included PKCα or PKCζ dominant negative construct over-expression or treatment with the PKCα or PKCζ pseudosubstrate inhibiting peptide; and PKCδ activation included wild type PKCδ adenoviral construct over-expression 1:100 titer (10⁷-10⁹ pfu) or with PKCδ specific activating agent such as bryostatin 10 nM or PKCδ RACK 100 nM. Cells were infected with a 1:100 titer (10⁷-10⁹ pfu) of a PKC dominant negative adenoviral construct for 1 h at 37° C. (rotation every 15 min). Thereafter cells were treated with the following selective PKC isoform modulating agents: PKCα pseudosubstrate inhibiting peptide of SEQ ID NO:1, adipsin (2 μg/ml), insulin (0.1 unit/ml), hIL-6 (0.2 μg/ml, TNFα (12 μg/ml), KGF (0.2 μg/ml), adiponectin (0.2 μg/ml), rosiglitazone (PPAR-γ agonist 100 nM), PKCδ RACK (100 nM) and Src inhibitors cocktail (100 nM). Photo documentation was performed as listed, on day 0, 24 and 48 hr post treatment. Magnification ×100. The results are summarized in Table 10.

In other experiments, primary skin keratinocytes were isolated and plated on non-coated dishes until confluence in low Ca²⁺ medium as described above. Cells were either infected with PKCα dominant negative adenoviral construct or treated with the PKCα pseudosubstrate inhibiting peptide of SEQ ID NO:1, or with wild type PKCδ adenoviral construct over-expression 1:100 titer (10⁷-10⁹ pfu) or with the specific PKCδ activating agent PKCδ RACK 100 nM. Then cells were either co-infected with adenoviral constructs or treated with PKC isoform modulating agents as follows: PKCζ, PKCε or PKCη inhibition included PKCζ, PKCε or PKCη dominant negative construct over-expression 1:100 titer (10⁷-10⁹ pfu) or treatment with PKCζ, PKCε or PKCη pseudosubstrate inhibiting peptide 1 μM; PKCζ or PKCη activation included wild type PKCζ or PKCη adenoviral construct over-expression 1:100 titer (10⁷-10⁹ pfu); PKCε activation included wild type PKCε adenoviral construct over-expression 1:100 titer or with specific activating agent such as PKCε RACK 100 nM. Cells were infected with a 1:100 titer (10⁷-10⁹ pfu) of a PKC dominant negative adenoviral construct for 1 h at 37° C. (rotation every 15 min). The results are summarized in Table 11.

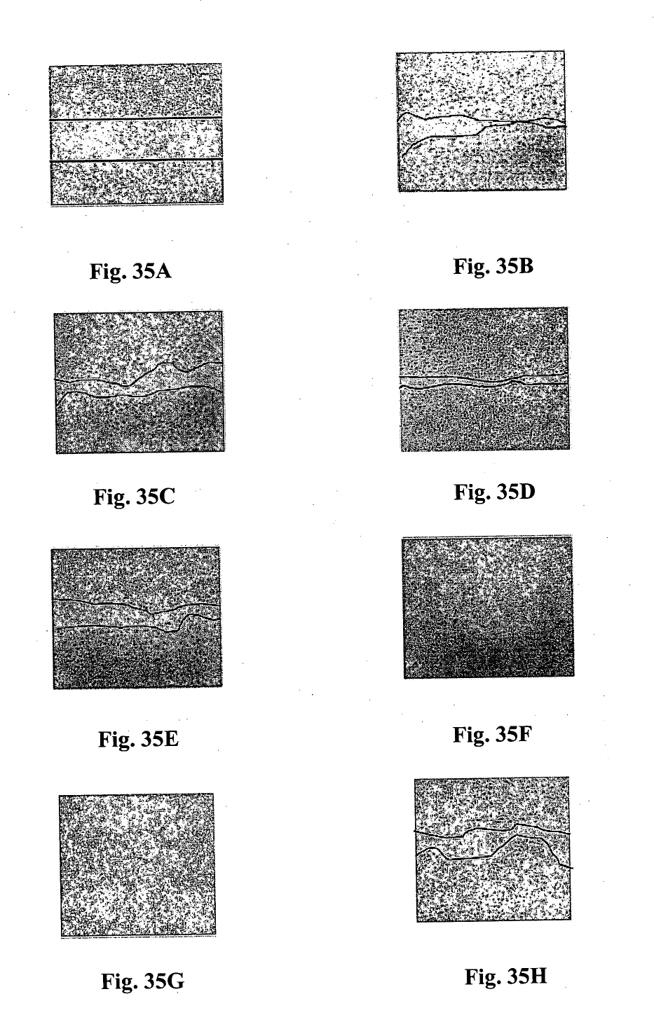

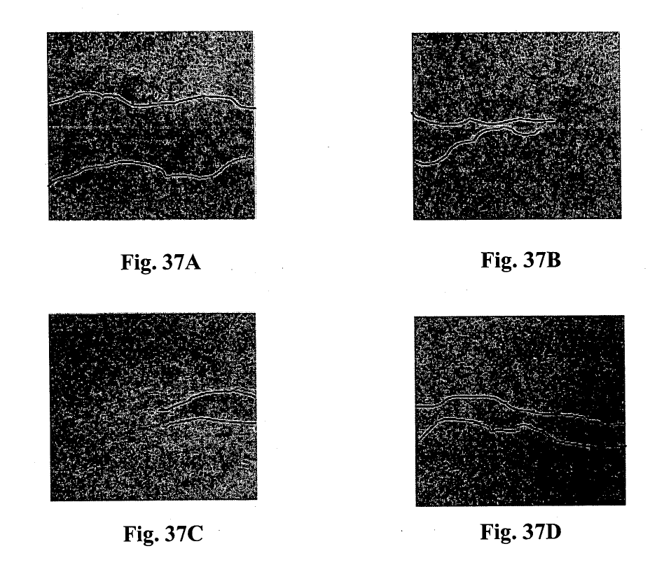

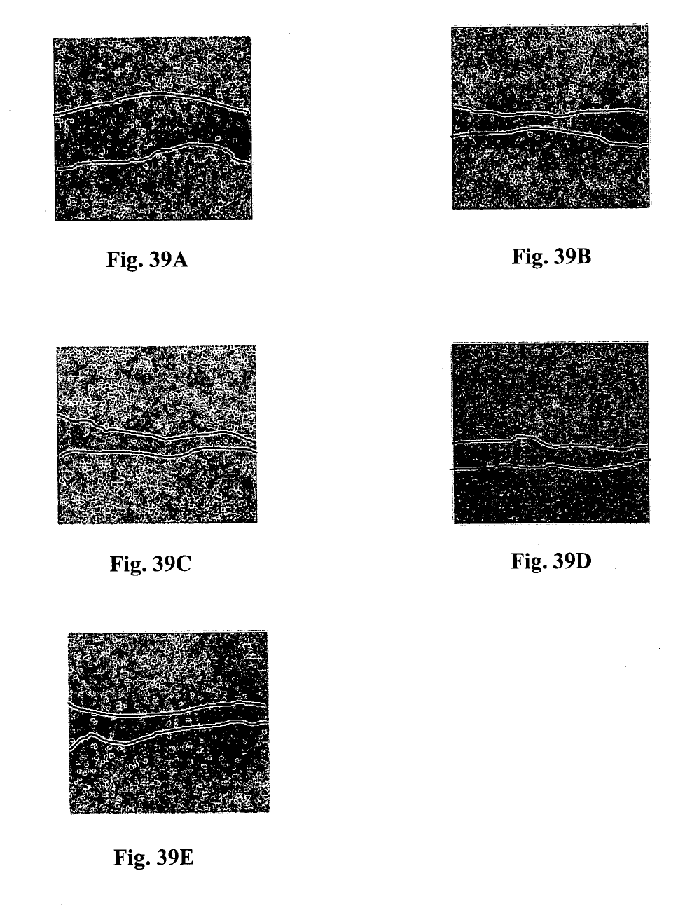

The effects of combination treatments on keratinocytes wound closure in vitro are summarized in Tables 10⁻¹¹. The results show that inhibition of expression and/or activity of PKCα in keratinocytes substantially promoted wound closure when combined with administering to the cells KGF, IL-6, TNFα or PKCδ RACK peptide (Table 10, wound closure index values of 6, 8, 10 and 8 respectively; FIGS. 35A-35C and 35G). Wound closure was also enhanced by the inhibition of PKCα combined with the stimulation of PKCη, PKCε or PKCζ in the cells (Table 11, wound closure index values of 10, 9 and 6, respectively; FIGS. 35A, 35D-35F and 35H). In addition, wound closure was promoted by the inhibition of PKCζ combined with administration of IL-6, TNFα or adiponectin to the cells (Table 10, wound closure index values of 9, 9 and 7, respectively; FIGS. 37A-37D). Further in addition, wound closure was accelerated by the promotion of PKCδ activity and/or expression combined with activating PKCε, activating PKCζ, or inhibiting PKCα in the cells, or by administration of adipsin to the cells (Table 11, wound closure index values of 7, 8 and 8; and Table 10, wound closure index value of 8, respectively; FIGS. 39A-39E).

TABLE 10
The effect of treatment combinations on the closure of keratinocytes
in vitro wounds¹

| | PKCα | PKCδ | PKCζ | |
|---|---|---|---|---|
| | inhibition | activation | inhibition | Insulin |
| Control | 3 | ND | 0 | ND |
| KGF | 6 | ND | 0 | ND |
| IL-6 | 8 | ND | 9 | ND |
| TNFα | 10 | ND | 9 | ND |
| Adipsin | 4 | 8 | ND | ND |
| Adiponectin | 2 | ND | 7 | ND |
| Rosiglitazone | 2 | ND | 2 | ND |
| SRC inhibitors | 0 | ND | 0 | ND |
| PKCδ RACK | 8 | ND | 0 | 9 |
| PKCα pseudosubstrate | ND | ND | ND | 9 |

¹Scale of closure was 0 (no closure) to 10 (complete closure).
ND = not determined.

TABLE 11
The effect of treatment combinations on the closure of keratinocytes
in vitro wounds¹

| | PKCα | PKCδ |
|---|---|---|
| | inhibition | activation |
| Control | 3 | 3 |
| PKCη activation | 10 | ND |
| PKCη inhibition | 5 | ND |
| PKCε activation | 9 | 7 |
| PKCε inhibition | 2 | 3 |
| PKCζ activation | 6 | 8 |
| PKCζ inhibition | 3 | 3 |
| PKCα inhibition | — | 8 |

¹Scale of closure was 0 (no closure) to 10 (complete closure).
ND = not determined.

Hence, the results indicate that wound closure can be substantially accelerated by modulating expression and/or activity of specific PKC isoforms in the dermal and epidermal cells colonizing the wound area, when combined with administering to the cells a growth factor such as KGF, a hormone such as insulin, an adipokine such as adipsin, adiponectin, IL-6 or TNFα, PKCδ RACK and/or GW9662.

### Example 29

### Copolymer-1 Inhibits PKCη Activity in Keratinocytes In Vitro

PKC Activity Assay was Performed as Described in Materials and Methods hereinabove. Plated primary keratinocytes were incubated with 2 concentrations of Copolymer-1 (Cop-1): 55 μg/kg and 5 μg/kg for either 10 minutes or 5 hours and specific PKCη activity was measured with the use of the SignaTECT Protein Kinase C Assay System (Promega, Madison, Wis., USA) according to the manufacturer's instructions.

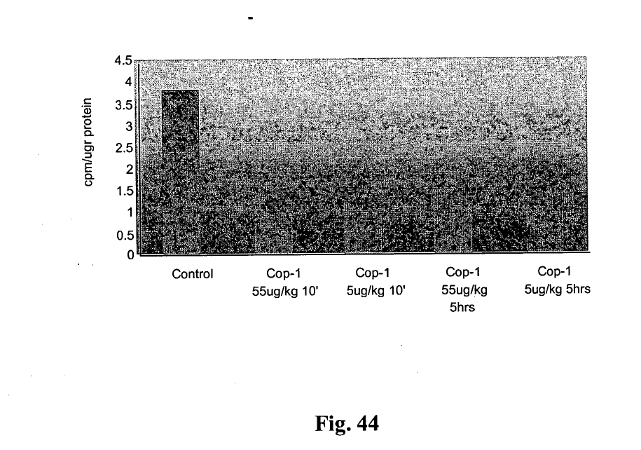

The results, presented in FIG. 44, indicate that Copolymer-1 inhibits PKCη activity with both concentrations tested and at both time points. Thus, it may be concluded that Copolymer-1 inflicts this inhibition as an immediate effect which can be sustained for a prolonged period of time. Since 55 μg/kg Cop-1 is the concentration used for the drug Copaxone® when treating human patients, the results further indicate that the inhibiting effect on PKCη in skin can be effective at 1/10 the dose for immediate as well as prolonged inhibition.

### Example 30

### Efficacy of Copolymer-1 in Wound Healing In Vitro and In Vivo

Materials and Methods

(i) Reagents.

Copolymer-1 was obtained from Teva Pharmaceutical Industries Ltd. (Petach Tikva, Israel). Insulin (Humulin) was purchased from Eli Lilly, USA; and PKCα pseudosubstrate inhibitor of SEQ ID NO:1 was obtained from CalbioChem (San Diego, Calif., USA).

(ii) In Vitro Assays.

Primary skin keratinocytes (dermal cells) were cultured for five days in Petri dishes (5 cm i.d.), and an artificial cross-over scratch was then formed in each dish with a 200 μl pipette tip. Thereafter, the cells were treated with either PBS (control), insulin (6.7×10⁻⁷M), Cop-1 (55 μg/dish) or a mixture of Cop-1 (55 μg/dish)+insulin (6.7×10⁻⁷M) The resulting wound closure levels were determined 48 hours following treatment using index values ranging from 0 (no closure) to 10 (complete closure).

(ii) In Vivo Assays.

Wounds were performed on the back of 8-10 week old C57BL mice by incision and were treated daily for 7 days with either PBS (control), insulin (6.7×⁻⁷ M), PKCα inhibitor of SEQ ID NO:1 (10⁻⁶M) (herein designated HO/02), Copaxone® (55 μg/ml), a mixture of Cop-1 and insulin or a mixture of Cop-1 and HO/02. At the indicated time points all mice were sacrificed and treated wounds were histologically analyzed for epidermal processes and the remodeling at the wound gap.

Histological assessment of the wounds was performed according to specific wound healing parameters such as epidermal closure. Wounds were considered epidermally closed when a complete distribution of Keratin 14 staining was evident across the wound gap. Keratin-14 represents basal layer formation of the newly formed epidermis.

Results

(i) In Vitro Assays.

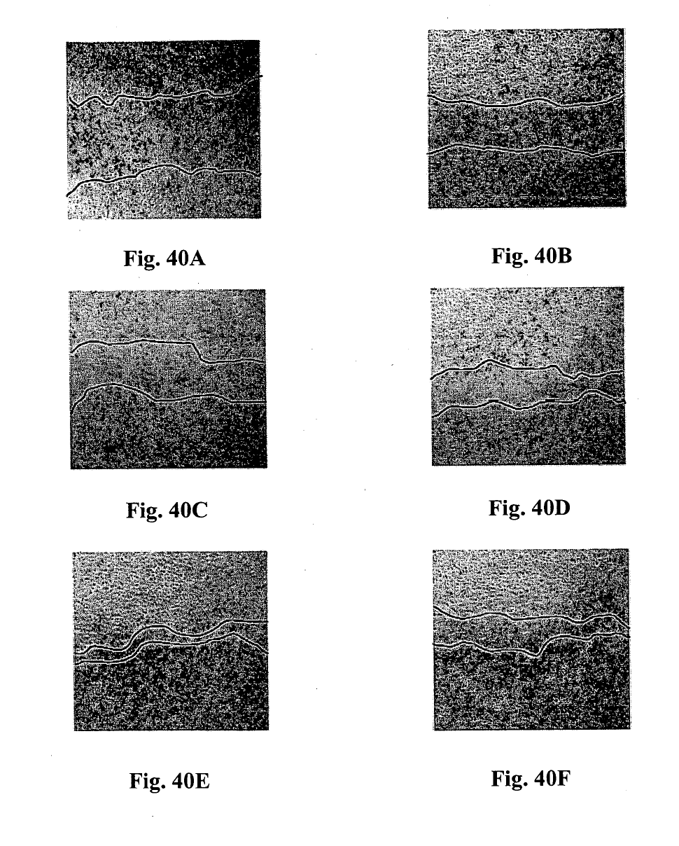

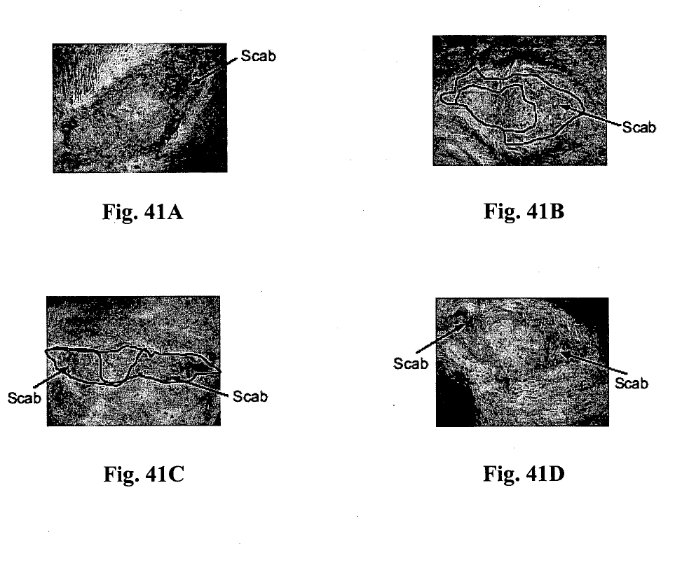

As illustrated in FIGS. 40A-40C and 40E, Cop-1 and insulin treatments had a similar effect on keratinocyte migration. However, the combination of Cop-1 and insulin promoted a synergistic effect on cell migration toward wound closure.

From the result it is clear that Cop-1 alone may be considered for as a single agent therapeutic for wound healing. In addition, it does serve as a prominent component for a combined formulation as seen in this experiment with insulin to strongly effect wound healing in keratinocytes.

(ii) In Vivo Assays.

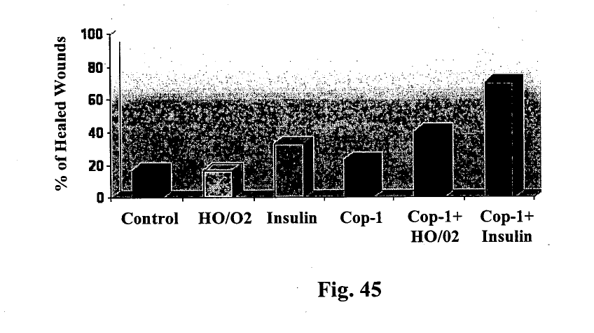

According the results, represented in FIG. 45, Cop-1 alone did not promote improved effect with respect to epidermal closure. The combination of Cop-1 with HO/02 produced an effect which was similar to the effect of insulin alone (only slightly better that controls). However, the combined formulation of insulin with Cop-1 exhibited a synergistic effect on the formation of a new epidermal layer to promote wound re-epithelialization.

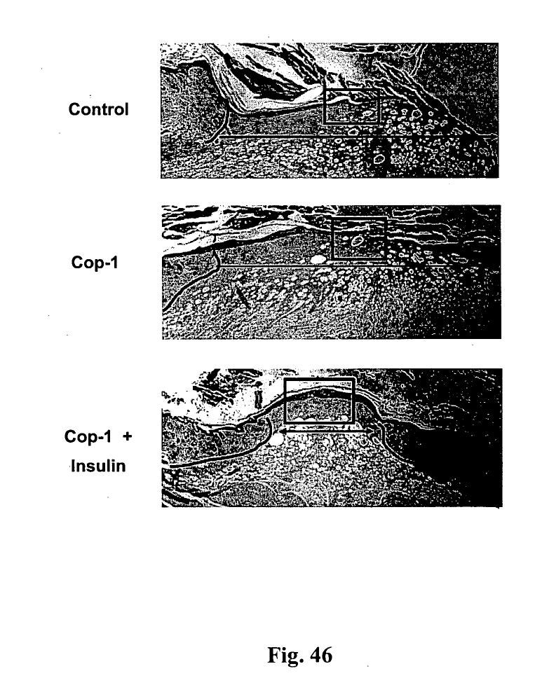

Histological presentation of wounds treated with Cop-1 and the combination of Cop-1 and insulin was performed utilizing H&E staining and presented in FIG. 46. These data clearly demonstrate the prominent effect of Cop-1 on wound healing in vivo. In all groups treated with Cop-1, the wound size was significantly smaller in comparison to control. Moreover, scab formation was augmented in all Cop-1 treated groups and oozing at the wounded area was reduced (not shown). Morphologically, the combination of Cop-1 with HO/02 further promoted reduction in wound size and scab detachment. In addition, histological analysis demonstrated that the combination of Cop-1 with insulin was superior to Cop-1 and HO/02 in relation to promotion of wound re-epithelialization. Therefore, Cop-1 combined with insulin may be even more promising for optimal closure and reconstruction of wounds.

Overall these data demonstrate the direct effect of Cop-1 on skin cells as well as on wound healing process, marking it a potential candidate for the treatment of acute wounds, especially in combination with insulin.

### Example 31

### Efficacy of Copolymer-1 in Combination with the Formulation of Insulin and the PKCα Inhibitor in Wound Healing In Vivo

Wounds were performed on the back of 8-10 week old C57BL mice by incision and were treated daily for 7 days with either PBS (control), insulin (Humulin, Eli Lilly, USA) (6.7×10⁻⁷M), PKCα inhibitor of SEQ ID NO:1 (10⁻⁶M) (Calbiochem, San Diego, Calif., USA) (herein designated HO/02), a combination of insulin and HO/02 (herein designated HO/03/03), Cop-1 (55 μg/ml), a mixture of Cop-1 and insulin, or a mixture of Cop-1 and HO/03/03. At the indicated time points all mice were sacrificed and treated wounds were histologically analyzed for epidermal processes and the remodeling at the wound gap.

Histological assessment of the wounds was performed by utilizing histological and immunohistochemical staining. Epidermal closure was assessed by Keratin 14 staining. When a complete distribution of staining was present over the wound gap, the wound was considered epidermally closed. Dermal contraction was assessed utilizing H&E staining. When both edges of the wound (normal dermis and or hair follicles) were confined to a fixed field in a microscope magnification of ×100, wounds were considered contracted. Granulation tissue formation was assessed utilizing H&E staining as well. Quantification of granulation tissue was calculated as percent of formed granulation tissue in the wound gap in a microscope magnification of ×100.

As shown in Table 12, treatment with Cop-1 alone provides a limited effect on wound healing parameters. However, combining Cop-1 either with insulin or with HO/03/03 formulation exhibit improved healing parameters promoting effective wound healing. Thus, it may be concluded that the combinations of Cop-1 with either insulin alone or the HO/03/03 formulation may serve as potential therapeutic formulations promoting wound healing in vivo.

TABLE 12
Wound healing efficacy of Copolymer-1 in combination with the
HO/O3/O3 formulation in wound healing in vivo

| | Epidermal | Dermal | |
|---|---|---|---|
| Treatment group | closure | contraction | Granulation tissue |
| Control | 0% | 0% | 55% |
| Cop-1 | 17% | 0% | 70% |
| Cop-1 + insulin | 40% | 40% | 82% |
| Cop-1 + HO/03/03 | 20% | 20% | 80% |
| HO/03/03 | 17% | 17% | 95% |

* Quantitative analyses of healing parameters were made as described above.
Results are presented as percent of wounds in each group.

### Example 32

### Influence of Thymus Secreted Substances on the Wound Healing Process

Full thickness skin incisions (20 mm) were performed on the upper back of anesthetized streptozotocin (STZ)-injected (175 mg/kg body weight) diabetic and non diabetic 8-10 week old C57BL mice. Wounds were treated daily for 7-9 days either with PBS or a formulation (herein designated HO/03/03) containing insulin (1 μM) and the N-myristoylated PKCα inhibitor (1 μM) of SEQ ID NO:1 The animals were sacrificed and the wounds were histologically analyzed for the presence of thymus in the proximity of the wound area as well as healing parameters including: epidermal and dermal closure utilizing staining procedure as described in Example 20 above.

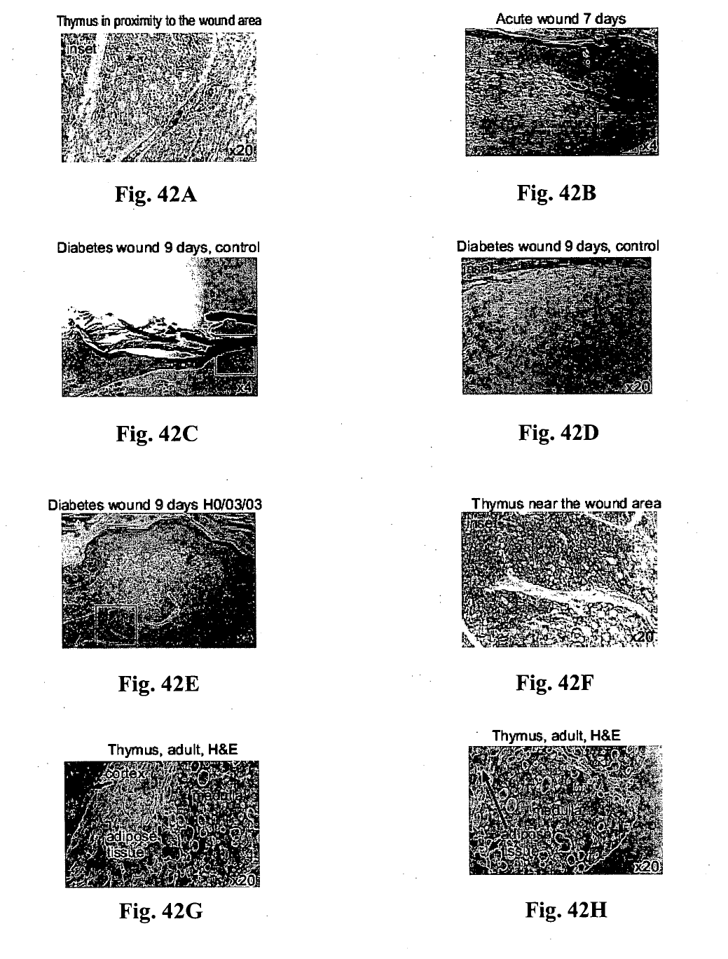

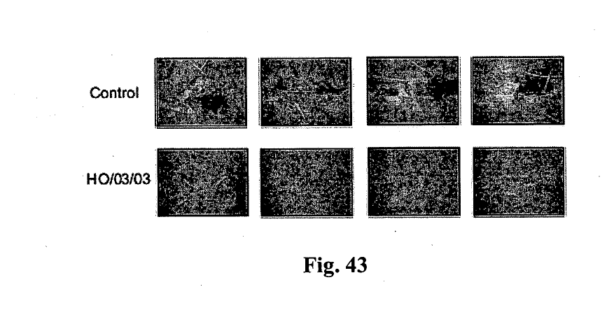

As can be seen in FIGS. 42A-42H, the presence of thymus in close proximity to the wound gap correlated with accelerated epithelization, granulation of tissue formation and dermal contraction in treated wounds. These observations indicate the possible role of thymus secreted substances in wound healing. Accordingly, thymus derived substances such as thymosin, beta thymosins (e.g., thymosin beta 4, thymosin beta 10, thymosin beta 9; thymosin beta 12, thymosin beta 14), alpha thimosins (e.g., thymosin alpha, 1/zadaxin, prothymosin alpha, parathymosin alpha), thymulin, IGFI, IGFII, NGF, somatostatin, thyroglobulin, parathyroid hormone and/or thymic hormonal peptides (THPs) may serve as potential healing agents in treatment for accelerating the wound healing process.

### Example 33

### Efficacy of the HO/03/03 Formulation on Acute and Chronic Wound Healing in Animals

Animals have a variety of common disease presentations that require wound management. Therefore, veterinary dermatology is one of the most rapidly growing disciplines in veterinary medicine. Wounds commonly found in animals, including horses, dogs, cats and livestock, generally heal by second intention (second intention means that the wounds are not surgically closed or pushed together so as the two sides of the wound come in contact with each other (this is first intention healing). Second intention is when wounds develop a granulation tissue to fill the gap and then promote closure). This process takes a long time, especially when the limbs are injured. In animals as well as in humans, a wound healing process may be further complicated by factors such as contamination, infection or skin degeneration.

Wound healing cases studied here included both acute and chronic wounds. Acute classification was addressed to post-surgical wounds in female dogs undergoing sterilization. Chronic classification was addressed to wounds in horses suffering from overabundance of granulation tissue in which proliferation of fibroblasts and angiogenesis are pathologically increased. This abnormal granulation tissue overgrows above a level of the epithelium thus disrupting adjacent skin to grow over the wound area. This specific wound type is designated ‘proud flesh’.

(i) Acute Wounds.

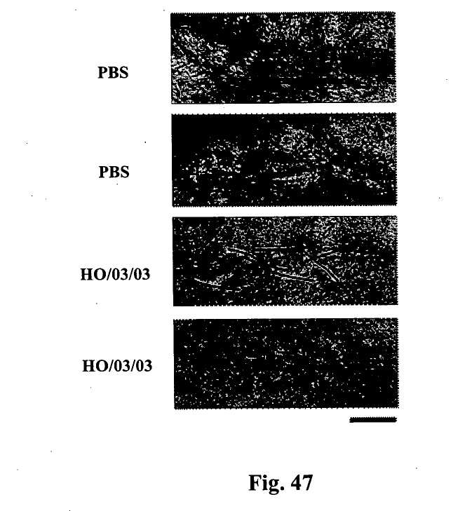

Four separate post surgical wounds of female dogs were treated daily with either PBS (control) or the HO/03/03 formulation (1 μg in PBS per 1 cm²). HO/03/03 formulation was applied on pad and padded topically to the wound. Each treatment was performed for 20 minutes. Photo-documentation representing wounds 5 days post surgery are shown in FIG. 47.

As can be observed from the results in FIG. 47, whereas PBS-treated wounds display edema and oozing from wound site, the wounds treated with HO/03/03 formulation exhibit a more progressive stage of healing.

(ii) Chronic Wounds.

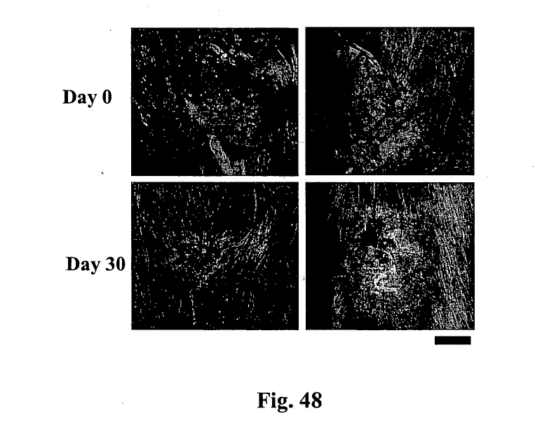

In order to test the efficacy of the HO/03/03 formulation for treating chronic wounds, 2 “proud flesh” chronic wounds that failed to heal for 4 months, located on separate limbs of a horse that had a history of recurring wounds for the past 5 years, were treated daily with HO/03/03 formulation (1 μg in PBS per 1 cm²). HO/03/03 was applied on pad gauze and bandaged to the wound using saran wrap. Each treatment was performed for 30 minutes. Following treatment, bandage was removed and wounds remained undressed (without covering with bandage). Results were photo-documented on day 30 of treatment and shown in FIG. 48.

As shown in FIG. 48, the PBS treated wounds are still oozing and display different degree of defects in granulation tissue (overabundant granulation tissue overgrows above the surrounding epithelium). However, following 30 days of treatment with the HO/03/03 formulation, the wounds display advanced healing parameters including reepithelialization with small regions covered with scabs and re-growth of fur around the wound area.

This case study suggests that HO/03/03 overcomes healing impaired wounds in “proud flesh” wounds by attenuating granulation tissue over-proliferation and out growth.

### Example 34

### Efficacy of HO/03/03 Formulation on Healing Burns in Animals

Veterinarians report many cases of severe burns in animals, usually dogs and cats. The classification of severe burns relates to burns in which at least 50% of the skin is damaged, and usually resulting in a prolonged healing time of over 6 months or even death, due to repeated infections and/or impaired homeostasis.

In this case study, a 30 kg female dog suffering from a severe burn of an unknown origin was treated. Prior to the treatment with the formulation HO/03/03, the dog was treated with antibiotics and irrigation of wound for a period of over 40 days. However, the wound failed to close and was subjected to recurring infections, to a stage that the dog developed immunity to the antibiotics, and veterinarian suggested putting the dog to sleep.

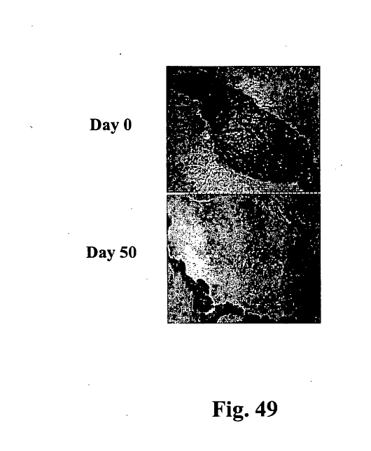

The wound was treated daily with the HO/03/03 formulation (1 μg in PBS per 1 cm²). HO/03/03 was applied on pad gauze and each treatment was performed for 20 minutes. Following treatment bandage was removed and wound was left unbandaged. Results were photo-documented and shown in FIG. 49.

The upper panel of FIG. 49 shows the severe burn wound, prior to the treatment with HO/03/03 formulation. However, as demonstrated in the lower panel of the figure, following 50 days of HO/03/03 treatment, complete healing was observed. Interestingly, during healing process, skin area did not contract but rather fully regenerated.

Based on the results of this study it may be concluded that HO/03/03 formulation induces quality healing of an extensive burn wound.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications mentioned in this specification are herein incorporated in their entirety by reference into the specification, to the same extent as if each individual publication, patent, patent application was specifically and individually indicated to be incorporated herein by reference. In addition, citation or identification of any reference in this application shall not be construed as an admission that such reference is available as prior art to the present invention.

### REFERENCES

Alessenko, A. Khan, W. A. Wetsel, W. C. Hannun, Y. A., Selective changes in protein kinase C isoenzymes in rat liver nuclei during liver regeneration, Biochem. Biophys. Res. Commun., 1992, 182, 1333-1339
Andresen, J. L. Ledet, T. Ehlers, N., Keratocyte migration and peptide growth factors: the effect of PDGF, bFGF, EGF, IGF-I, aFGF and TGF-beta on human keratocyte migration in a collagen gel, Curr. Eye Res., 1997, 16, 605-613
Bajou, K. Noel, A. Gerard, R. D. Masson, V. Brunner, N. Holst-Hansen, C. Skobe, M. Fusenig, N. E. Carmeliet, P. Collen, D. Foidart, J. M., Absence of host plasminogen activator inhibitor 1 prevents cancer invasion and vascularization, Nat. Med., 1998, 4, 923-928
Bandyopadhyay, G. Standaert, M. L. Kikkawa, U. On o, Y. Moscat, J. Farese, R. V., Effects of transiently expressed atypical (zeta, lambda), conventional (alpha, beta) and novel (delta, epsilon) protein kinase C isoforms on insulin-stimulated translocation of epitope-tagged GLUT4 glucose transporters in rat adipocytes: specific interchangeable effects of protein kinases C-zeta and C-lambda, Biochem. J., 1999, 337, 461-470
Benes, C. H., Ning Wu, Andrew E. H. Elia, Tejal Dharia, Lewis C. Cantley, Stephen P. Soltoff. The C2 domain of PKCδ is a phosphotyrosine binding domain, Cell, 2005, 121, 271-280.
Braiman, L. Alt, A. Kuroki, T. Ohba, M. Bak, A. Tennenbaum, T. Sampson, S. R., Protein kinase Cdelta mediates insulin-induced glucose transport in primary cultures of rat skeletal muscle, Mol. Endocrinol., 1999a, 13, 2002-2012
Braiman, L. Sheffi-Friedman, L. Bak, A. Tennenbaum, T. Sampson, S. R., Tyrosine phosphorylation of specific protein kinase C isoenzymes participates in insulin stimulation of glucose transport in primary cultures of rat skeletal muscle, Diabetes, 1999b, 48, 1922-1929
Chida, K. Sagara, H. Suzuki, Y. Murakami, A. Osada, S. Ohno, S. Hirosawa, K. Kuroki, T., The η isoform of protein kinase C is localized on rough endoplasmic reticulum, Mol. Cell Biol., 1994, 14, 3782-3790
Coghlan, M. P. Pillay, T. S. Tavare, J. M. Siddle, K., Site-specific anti-phosphopeptide antibodies: use in assessing insulin receptor serine/threonine phosphorylation state and identification of serine-1327 as a novel site of phorbol ester-induced phosphorylation, Biochem. J., 1994, 303, 893-899
Denning, M. F. Dlugosz, A. A. Williams, E. K. Szallasi, Z. Blumberg, P. M. Yuspa, S. H., Specific protein kinase C isozymes mediate the induction of keratinocyte differentiation markers by calcium, Cell Growth Differ., 1995, 6, 149-157
Dlugosz, A. A. Pettit, G. R. Yuspa, S. H., Involvement of Protein kinase C in Ca²⁺-mediated differentiation on cultured primary mouse keratinocytes, J. Invest. Dermatol., 1990, 94, 519-519
Dlugosz, A. A. Yuspa, S. H., Coordinate changes in gene expression which mark the spinous to granular cell transition in epidermis are regulated by protein kinase C, J. Cell Biol., 1993, 120, 217-225
Dlugosz, A. A. Glick, A. B. Tennenbaum, T. Weinberg, W. C. Yuspa, S. H., Isolation and utilization of epidermal keratinocytes for oncogene research. In: P. K. Vogt and I. M. Verma (eds.), Methods in Enzymology, 1995, 3-20, New York: Academic Press
Formisano, P. Oriente, F. Miele, C. Caruso, M. Auricchio, R. Vigliotta, G. Condorelli, G. Beguinot, F., In NIH-3T3 fibroblasts, insulin receptor interaction with specific protein kinase C isoforms controls receptor intracellular routing, J. Biol. Chem., 1998, 273, 13197-13202
Fuchs, E., Epidermal differentiation: the bare essentials, J. Cell Biol., 1990, 111, 2807-2814
Greenhalgh, D. A. Rothnagel, J. A. Roop, D. R., Epidermis: An attractive target tissue for gene therapy, J. Invest. Dermatol., 1994, 103, 63S-69S
Grunfeld, C., Diabetic foot ulcers: etiology, treatment, and prevention, Adv. Intern. Med., 1992, 37, 103-132
Gschwendt, M., Protein kinase C delta, Eur. J. Biochem., 1999, 259, 555-564
Hennings, H. Michael, D. Cheng, C. Steinert, P. Holbrook, K. Yuspa, S. H., Calcium regulation of growth and differentiation of mouse epidermal cells in culture, Cell, 1980, 19, 245-254
Hennings, H. Holbrook, K. A., Calcium regulation of cell-cell contact and differentiation of epidermal cells in culture. An ultrastructural study, Exp. Cell Res., 1983, 143, 127-142
Hofmann, J. The potential for isoenzyme-selective modulation of protein kinase C, The FASEB J, 1997, 11, 649-669.
Kiritsy, C. P. Lynch, B. Lynch, S. E., Role of growth factors in cutaneous wound healing: a review, Crit. Rev. Oral Biol. Med., 1993, 4, 729-760
Knighton, D. R. Fiegel, V. D., Growth factors and comprehensive surgical care of diabetic wounds, Curr. Opin. Gen. Surg., 1993, 32-39
Kuroki, T. Kashiwagi, M. Ishino, K. Huh, N. Ohba, M., Adenovirus-mediated gene transfer to keratinocytes—a review, J. Investig. Dermatol. Symp. Proc., 1999, 4, 153-157
Mischak, H. Pierce, J. H. Goodnight, J. Kazanietz, M. G. Blumberg, P. M. Mushinski, J. F., Phorbol ester-induced myeloid differentiation is mediated by protein kinase C-α and -δ and not by protein kinase C-βII, -ε, -zeta and eta, J. Biol. Chem., 1993a, 268, 20110-20115
Mischak, H. Goodnight, J. Kolch, W. Martiny-Baron, G. Schaechtle, C. Kazanietz, M. G., Blumberg, P. M. Pierce, J. H. Mushinski, J. F., Over-expression of protein kinase C-δ and -ε in NIH 3T3 cells induces opposite effects of growth, morphology, anchorage dependence, and tumorigenicity, J. Biol. Chem., 1993b, 268, 6090-6096
Miyake, S. Makimura, M. Kanegae, Y. Harada, S. Sato, Y. Takamori, K. Tokuda, C. Saito, I., Efficient generation of recombinant adenoviruses using adenovirus DNA-terminal protein complex and a cosmid bearing the full-length virus genome, Proc. Natl. Acad. Sci. U.S.A., 1996, 93, 1320-1324
Nishizuka, Y., The molecular heterogeneity of PKC and its implications for cellular regulation, Nature, 1988, 334, 661-665
Nishizuka, Y., The family of protein kinase C for signal transduction, JAMA, 1989, 262, 1826-1833
Ohba, M. Ishino, K. Kashiwagi, M. Kawabe, S. Chida, K. Huh, N. H. Kuroki, T., Induction of differentiation in normal human keratinocytes by adenovirus-mediated introduction of the eta and delta isoforms of protein kinase C, Mol. Cell Biol., 1998, 18, 5199-5207
Osada, S. Mizuno, K. Theo, T. C. Akita, Y. Suzuki, K. Kuroki, T. Ohno, S., A phorbol ester receptor/protein kinase, nPKCₙ, a new member of the protein kinase C family predominantly expressed in lung and skin, J. Biol. Chem., 1990, 265, 22434-22440
Reiber, G. E. Lipsky, B. A. Gibbons, G. W., The burden of diabetic foot ulcers, Am. J. Surg., 1998, 176, 5S-S10
Reynolds N J et al., Br J Dermatol. 1995 133(2), 157-67,
Rosenfeld, M. A. Siegfried, W. Yoshimura, K. Yoneyama, K. Fukayama, M. Stier, L. E. Paakko, P. K. Gi, P. Stratford-Perricaudet, M. Jallet, J. Pavirani, A. Lecocq, J. P. Crystal, R. G., Adenovirus-mediated transfer of a recombinant al-antitrypsin gene to the lung epithelium in vivo, Science, 1991, 252, 431-434
Setoguchi, Y. Jaffe, H. A. Danel, C. Crystal, R. G., Ex Vivo and in vivo gene transfer to the skin using replication-deficient recombinant adenovirus vectors, J. Invest. Dermatol., 1994, 102, 415-421
Shaw, J. E. Boulton, A. J., The pathogenesis of diabetic foot problems: an overview, Diabetes, 1997, 46 Suppl 2: S58-61
Singer, A. J. Clark, R. A., Cutaneous wound healing, N. Engl. J. Med., 1999, 341, 738-746
Soltoff, S. P. Toker, A., Carbachol, substance P and phorbol ester promote the tyrosine phosphorylation of protein kinase Cδ in salivary gland epithelial cells, J. Biol. Chem., 1995, 270, 13490-13495
Sun, Q. Tsutsumi, K. Kelleher, M. B. Pater, A. Pater, M. M., Squamous metaplasia of normal and carcinoma in situ of HPV 16-immortalized human endocervical cells, Cancer Res., 1992, 52, 4254-4260
Tennenbaum, T. Li, L. Belanger, A. J. De Luca, L. M. Yuspa, S. H., Selective changes in laminin adhesion and α6β4 integrin regulation are associated with the initial steps in keratinocyte maturation, Cell Growth Differ., 1996a, 7, 615-628
Tennenbaum, T. Belanger, A. J. Quaranta, V. Yuspa, S. H., Differential regulation of integrins and extracellular matrix binding in epidermal differentiation and squamous tumor progression, J. Invest. Dermatol., 1996b, 1, 157-161
Threadgill, D. W. Dlugosz, A. A. Hansen, L. A. Tennenbaum, T. Lichti, U. Yee, D. LaMantia, C. Mourton, T. Herrup, K., Harris, R. C., Barnard, J. A., Yuspa, S. H., Coffey, R. J. Magnuson, T., Targeted disruption of mouse EGF receptor: effect of genetic background on mutant phenotype, Science, 1995, 269, 230-234
Wang, Q. J. Bhattacharyya, D. Garfield, S. Nacro, K. Marquez, V. E. Blumberg, P. M., Differential localization of protein kinase C delta by phorbol esters and related compounds using a fusion protein with green fluorescent protein, J. Biol. Chem., 1999, 274, 37233-37239
Weinstein, M. L., Update on wound healing: a review of the literature, Mil. Med., 1998, 163, 620-624
Werner, S. Breeden, M. Hubner, G. Greenhalgh, D. G. Longaker, M. T., Induction of keratinocyte growth factor expression is reduced and delayed during wound healing in the genetically diabetic mouse, Invest. Dermatol., 1994, 103, 469-473
Wertheimer, E. Trebicz, M. Eldar, T. Gartsbein, M. Nofeh-Mozes, S. Tennenbaum, T., Differential Roles of Insulin Receptor and Insulin-Like Growth Factor-1 Receptor in Differentiation of Murine Skin Keratinocytes, J. Invest. Dermatol., 2000
Whitby, D. J. Ferguson, M. W., Immunohistochemical localization of growth factors in fetal wound healing, Dev. Biol., 1991, 147, 207-215
Yuspa, S. H. Kilkenny, A. E. Steinert, P. M. Roop, D. R., Expression of murine epidermal differentiation markers is tightly regulated by restricted extracellular calcium concentrations in vitro, J. Cell Biol., 1989, 109, 1207-1217
Yuspa, S. H., The pathogenesis of squamous cell cancer: lessons learned from studies of skin carcinogenesis—Thirty-third G.H.A. Clowes Memorial Award Lecture, Cancer Res., 1994, 54, 1178-1189

1. A method of inducing or accelerating a healing process of a damaged skin or skin wound, the method comprising modulating expression and/or activity of at least two PKC isoforms in skin cells colonizing the damaged skin or skin wound area, to thereby induce or accelerate the healing process of the damaged skin or skin wound.

2. The method of claim 1, wherein said PKC isoforms are selected from the group consisting of PKCα, PKCβ, PKCδ, PKCε, PKCη, PKCζ, PKCγ, PKCθ, PKCλ and PKCι.

3. The method of claim 2, wherein said PKC isoforms are selected from the group consisting of PKCα, PKCβ, PKCδ, PKCε, PKCη and PKCζ.

4. The method of claim 1, comprising the step of administering to the damaged skin or skin wound area therapeutically effective amounts of at least two PKC isoform modulating agents, each agent capable of modulating the production and/or activity of each of said at least two PKC isoforms.

5. The method of claim 4, wherein at least one of said at least two PKC isoform modulating agents is a PKC isoform inhibitor.

6. The method of claim 5, wherein said PKC isoform inhibitor is a PKC isoform pseudosubstrate inhibitor, a peptide binding to the PKC isoform substrate region or Copolymer-1.

7. The method of claim 6, wherein said PKC isoform is PKCα and the PKCα inhibitor is the N-myristoylated PKCα pseudosubstrate peptide of SEQ ID NO: 1.

8. The method of claim 6, wherein said PKC isoform is PKCα and the PKCα inhibitor is a PKCα pseudosubstrate inhibitor selected from the group of peptides consisting of SEQ ID NO: 2 to SEQ ID NO: 7, or a peptide binding to the PKCα substrate region selected from the group of peptides consisting of SEQ ID NO: 8 to SEQ ID NO: 24.

9. The method of claim 6, wherein said PKC isoform is PKCη and the PKCη inhibitor is Copolymer-1.

10. The method of claim 6, wherein said PKC isoform is PKCη and the PKCη inhibitor is the N-myristoylated PKCη pseudosubstrate peptide of SEQ ID NO: 25.

11. The method of claim 6, wherein said PKC isoform is PKCTη and the PKCη inhibitor is a peptide binding to the PKCη substrate region selected from the peptides of SEQ ID NO: 26 and SEQ ID NO: 27.

12. The method of claim 6, wherein said PKC isoform is PKCβ and the PKCβ inhibitor is a peptide binding to the PKCβ substrate region selected from the group of peptides consisting of SEQ ID NO: 28 to SEQ ID NO: 38.

13. The method of claim 6, wherein said PKC isoform is PKCζ and the PKCζ inhibitor is a peptide binding to the PKCζ substrate region selected from the group of peptides consisting of SEQ ID NO: 39 to SEQ ID NO: 43.

14. The method of claim 4, wherein at least one of said at least two PKC isoform modulating agents is a PKC isoform activator.

15. The method of claim 14, wherein said PKC isoform activator is a peptide binding to a PKC isoform substrate region, a peptide acting on a PKC isoform phosphorylation site, insulin, a growth factor, bryostatin, a PKC isoform RACK peptide or a MARCKS-derived peptide.

16. The method of claim 15, wherein said PKC isoform is PKCδ and the PKCδ activator is insulin.

17. The method of claim 15, wherein said PKC isoform is PKCδ and the PKCδ activator is a peptide binding to the PKCδ substrate region selected from the group of peptides consisting of SEQ ID NO: 44 to SEQ ID NO: 51; a peptide acting on the PKCδ phosphorylation site selected from the group of peptides consisting of SEQ ID NO: 52 to SEQ ID NO: 54; a PKCδ RACK peptide; or a peptide corresponding to the C2 domain of PKCδ.

18. The method of claim 15, wherein said PKC isoform is PKCε and the PKCε activator is a PKCε RACK peptide.

19. The method of claim 15, wherein said PKC isoform is PKCζ and the PKCζ activator is the MARCKS-derived peptide of SEQ ID NO: 55.

20. The method of claim 15, wherein said PKC isoform activator is a growth factor selected from the group consisting of PDGF, KGF, EGF, TGF-β, ECGF and IGF1.

21. The method of claim 4, wherein at least one of said at least two PKC isoform modulating agents is a growth factor, an adipokine, Copolymer-1 or a PPAR-γ antagonist.

22. The method of claim 21, wherein said growth factor is PDGF, KGF, EGF, TGF-β, ECGF or IGF1, and said PPAR-γ antagonist is GW9662.

23. The method of claim 21, wherein said adipokine is selected from the group consisting of adipsin, adiponectin, apelin, visfatin, resistin, leptin, lipoprotein lipase, plasminogen activator inhibitor-1 (PAI-1), TNF-α, IL-6, IL-4, IL-1β, angiotensin I to angiotensin IV and cycloanalogues thereof, angiotensinogen, 1-butyrylglycerol, matrix metalloproteinase 2, matrix metalloproteinase 9, and vascular endothelial growth factor.

24. The method of claim 1, wherein expression and/or activity of at least one of said at least two PKC isoforms is inhibited.

25. The method of claim 24, wherein said PKC isoform that is inhibited is PKCα, PKCβ, PKCη or PKCζ.

26. The method of claim 24, wherein expression and/or activity of two PKC isoforms is inhibited.

27. The method of claim 26, wherein said PKC isoforms that are inhibited are PKCα and PKCη.

28. The method of claim 1, wherein expression and/or activity of at least one of said at least two PKC isoforms is activated.

29. The method of claim 28, wherein said PKC isoform that is activated is PKCδ, PKCε or PKCζ.

30. The method of claim 1, wherein expression and/or activity of PKCα is inhibited and expression and/or activity of PKCδ is activated.

31. The method of claim 1, wherein expression and/or activity of PKCη is inhibited and expression and/or activity of PKCδ is activated.

32. The method of claim 1, wherein expression and/or activity of both PKCα and PKCη are inhibited and expression and/or activity of PKCδ is activated.

33. The method of claim 1, wherein inhibition of at least one of said at least two PKC isoforms is achieved by a small interfering RNA (siRNA) molecule or by infecting said skin cells with a dominant-negative PKC isoform adenoviral construct.

34. The method of claim 1, wherein activation of at least one of said at least two PKC isoforms is achieved by infecting said skin cells with a wild-type PKC isoform adenoviral construct.

35. A method of inducing or accelerating a healing process of a damaged skin or skin wound, the method comprising inhibiting expression and/or activity of PKCα and activating expression and/or activity of PKCδ in skin cells colonizing the damaged skin or skin wound area, to thereby induce or accelerate the healing process of the damaged skin or skin wound.

36. The method of claim 35, comprising the step of administering to the damaged skin or skin wound area therapeutically effective amounts of a PKCαinhibitor and of a PKCδ activator, to thereby induce or accelerate the healing process of the damaged skin or skin wound.

37. The method of claim 36, wherein said PKCα inhibitor is the N-myristoylated PKCα pseudosubstrate peptide of SEQ ID NO: 1 and said PKCδ activator is insulin.

38. The method of claim 37, wherein said N-myristoylated PKCα pseudosubstrate peptide of SEQ ID NO: 1 and insulin are contained in a pharmaceutical composition adapted for topical application.

39. The method of claim 37, wherein said insulin is recombinant.

40. The method of claim 37, wherein said insulin is of a natural source.

41. The method of claim 37, wherein said insulin is administered in a therapeutically effective concentration ranging from 0.1 μM to 10 μM.

42. A method of inducing or accelerating a healing process of a damaged skin or skin wound, the method comprising administering therapeutically effective amounts of the N-myristoylated PKCα pseudosubstrate peptide of SEQ ID NO: 1 and insulin to the damaged skin or skin wound area, to thereby induce or accelerate the healing process of the damaged skin or skin wound.

43. The method of claim 4, comprising administering therapeutically effective amounts of a PKCη inhibitor and of a PKCδ activator, to thereby induce or accelerate the healing process of the damaged skin or skin wound.

44. The method of claim 43, wherein said PKCη inhibitor is Copolymer-1 or the N-myristoylated PKCη pseudosubstrate peptide of SEQ ID. NO: 25, and said PKCδ activator is insulin.

45. The method of claim 4, comprising administering therapeutically effective amounts of a PKCα inhibitor and a PKCη inhibitor, to thereby induce or accelerate the healing process of the damaged skin or skin wound.

46. The method of claim 45, wherein said PKCη inhibitor is the N-myristoylated PKCα pseudosubstrate peptide of SEQ ID NO: 1, and said PKCη inhibitor is Copolymer-1 or the N-myristoylated PKCη pseudosubstrate peptide of SEQ ID NO: 25.

47. The method of claim 4, comprising administering therapeutically effective amounts of a PKCα inhibitor, a PKCη inhibitor, and a PKCδ activator to thereby induce or accelerate the healing process of the damaged skin or skin wound.

48. The method of claim 47, wherein said PKCα inhibitor is the N-myristoylated PKCα pseudosubstrate peptide of SEQ ID NO: 1, said PKCη inhibitor is Copolymer-1, and said PKCδ activator is insulin.

49. A method of inducing or accelerating a healing process of a damaged skin or skin wound, the method comprising administering therapeutically effective amounts of the N-myristoylated PKCα pseudosubstrate peptide of SEQ ID NO: 1, Copolymer-1 and insulin to the damaged skin or skin wound area, to thereby induce or accelerate the healing process of the damaged skin or skin wound.

50. The method of claim 1, wherein said skin wound is selected from the group consisting of an ulcer, a diabetes related wound, a burn, a sun burn, an aging skin wound, a corneal ulceration wound, an inflammatory gastrointestinal tract disease wound, a bowel inflammatory disease wound, a Crohn's disease wound, an ulcerative colitis, a hemorrhoid, an epidermolysis bulosa wound, a skin blistering wound, a psoriasis wound, an animal skin wound, a proud flesh wound, an animal diabetic wound, a retinopathy wound, an oral wound (mucositis), a vaginal mucositis wound, a gum disease wound, a laceration, a surgical incision wound and a post surgical adhesions wound.

51. The method of claim 50, wherein said ulcer is selected from the group consisting of a diabetic ulcer, a pressure ulcer, a venous ulcer, a gastric ulcer and an HIV-related ulcer.

52. A pharmaceutical composition for topical application for inducing or accelerating a healing process of a damaged skin or skin wound, comprising a pharmaceutically acceptable carrier and a therapeutically effective amount of at least two agents, each of them capable of modulating the production and/or activity of one PKC isoform in the damaged skin or skin wound area.

53. The pharmaceutical composition of claim 52, wherein said PKC isoform is selected from the group consisting of PKCα, PKCβ, PKCδ, PKCε, PKCη, PKCζ, PKCγ, PKCθ, PKCλ and PKCι.

54. The pharmaceutical composition of claim 53, wherein said PKC isoform is selected from the group consisting of PKCα, PKCβ, PKCδ, PKCε, PKCη, and PKCζ.

55. The pharmaceutical composition of claim 52, wherein at least one of said at least two agents is a PKC isoform inhibitor.

56. The pharmaceutical composition of claim 55, wherein said PKC isoform inhibitor is a PKC isoform pseudosubstrate inhibitor, a peptide binding to the PKC isoform substrate region or Copolymer-1.

57. The pharmaceutical composition of claim 56, wherein said PKC isoform is PKCα and the PKCα inhibitor is the N-myristoylated PKCα pseudosubstrate peptide of SEQ ID NO: 1.

58. The pharmaceutical composition of claim 56, wherein said PKC isoform is PKCα and the PKCα inhibitor is a PKCα pseudosubstrate inhibitior selected from the group of peptides consisting of SEQ ID NO: 2 to SEQ ID NO: 7, or a peptide binding to the PKCα substrate region selected from the group of peptides consisting of SEQ ID NO: 8 to SEQ ID NO: 24.

59. The pharmaceutical composition of claim 56, wherein said PKC isoform is PKCη and the PKCη inhibitor is copolymer-1.

60. The pharmaceutical composition of claim 56, wherein said PKC isoform is PKCη and the PKCη inhibitor is the N-myristoylated PKCη pseudosubstrate peptide of SEQ ID NO: 25.

61. The pharmaceutical composition of claim 56, wherein said PKC isoform is PKCη and the PKCη inhibitor is a peptide binding to the PKCη substrate region selected from the peptide of SEQ ID NO: 26 or the peptide of SEQ ID NO: 27.

62. The pharmaceutical composition of claim 56, wherein said PKC isoform is PKCβ and the PKCβ inhibitor is a peptide binding to the PKCβ substrate region selected from the group of peptides consisting of SEQ ID NO: 28 to SEQ ID NO: 38.

63. The pharmaceutical composition of claim 47, wherein said PKC isoform is PKCζ and the PKCζ inhibitor is a peptide binding to the PKCζ substrate region selected from the group of peptides consisting of SEQ ID NO: 39 to SEQ ID NO: 43.

64. The pharmaceutical composition of claim 52, wherein at least one of said at least two agents is a PKC isoform activator.

65. The pharmaceutical composition of claim 64, wherein said PKC isoform activator is a peptide binding to the PKC isoform substrate region, a peptide acting on the PKC isoform phosphorylation site, insulin, a growth factor, bryostatin, a PKC isoform RACK peptide or a MARCKS-derived peptide.

66. The pharmaceutical composition of claim 65, wherein said PKC isoform is PKCδ and the PKCδ activator is insulin.

67. The pharmaceutical composition of claim 65, wherein said PKC isoform is PKCδ and the PKCδ activator is a peptide binding to the PKCδ substrate region selected from the group of peptides consisting of SEQ ID NO: 44 to SEQ ID NO: 51; a peptide acting on the PKCδ phosphorylation site selected from the group of peptides consisting of SEQ ID NO: 52 to SEQ ID NO: 54; a PKCδ RACK peptide; or a peptide corresponding to the C2 domain of PKC6.

68. The pharmaceutical composition of claim 65, wherein said PKC isoform is PKCε and the PKCε activator is a PKCε RACK peptide.

69. The pharmaceutical composition of claim 65, wherein said PKC isoform is PKCζ and the PKCζ activator is the MARCKS-derived peptide of SEQ ID NO: 55.

70. The pharmaceutical composition of claim 56, wherein said PKC isoform activator is a growth factor selected from the group consisting of PDGF, KGF, EGF, TGF-β, ECGF and IGF1.

71. The pharmaceutical composition of claim 52, wherein at least one of said at least two agents is a growth factor, Copolymer-1, an adipokine or a PPAR-γ antagonist.

72. The pharmaceutical composition of claim 71, wherein said growth factor is PDGF, KGF, EGF, TGF-β, ECGF or IGF1, and said PPAR-γ antagonist is GW9662.

73. The pharmaceutical composition of claim 71, wherein said adipokine is selected from the group consisting of adipsin, adiponectin, apelin, visfatin, resistin, leptin, lipoprotein lipase, plasminogen activator inhibitor-1 (PAI-1), TNF-α, IL-6, IL-4, IL-1β, angiotensin I to angiotensin IV and cycloanalogues thereof, angiotensinogen, 1-butyrylglycerol, matrix metalloproteinase 2, matrix metalloproteinase 9, and vascular endothelial growth factor.

74. The pharmaceutical composition of claim 52, wherein at least one of the at least two agents inhibits the production and/or activity of one PKC isoform.

75. The pharmaceutical composition of claim 74, wherein said PKC isoform that is inhibited is PKCα, PKCβ, PKCη or PKCζ.

76. The pharmaceutical composition of claim 52, wherein the two agents inhibit the production and/or activity of two PKC isoforms.

77. The pharmaceutical composition of claim 74, wherein one agent inhibits PKCα and the other agent inhibits PKCη.

78. The pharmaceutical composition of claim 52, wherein at least one of the at least two agents activates the production and/or activity of one PKC isoform.

79. The pharmaceutical composition of claim 67, wherein said PKC isoform that is activated is PKCδ, PKCε or PKCζ.

80. The pharmaceutical composition of claim 52, wherein one agent is a PKCα inhibitor and another agent is a PKCδ activator.

81. The pharmaceutical composition of claim 80, wherein the PKCα inhibitor is the N-myristoylated PKCα pseudosubstrate peptide of SEQ ID NO: 1 and the PKCδ activator is insulin.

82. The pharmaceutical composition of claim 43, wherein one agent is a PKCη inhibited and another agent is a PKCδ activator.

83. The pharmaceutical composition of claim 82, wherein the PKCη inhibitor is Copolymer-1 and the PKCδ activator is insulin.

84. The pharmaceutical composition of claim 43, wherein one agent is a PKCα inhibitor and another agent is a PKCη inhibitor.

85. The pharmaceutical composition of claim 84, wherein the PKCα inhibitor is the N-myristoylated PKCα pseudosubstrate peptide of SEQ ID NO: 1 and the PKCη inhibitor is Copolymer 1.

86. The pharmaceutical composition of claim 43, wherein one agent is a PKCα inhibitor, another agent is a PKCη inhibitor and a third agent is a PKCδ activator.

87. The pharmaceutical composition of claim 86, wherein the PKCα inhibitor is the N-myristoylated PKCα pseudosubstrate peptide of SEQ ID NO: 1, the PKCη inhibitor is Copolymer 1 and the PKCδ activator is insulin.

88. A pharmaceutical composition for topical application for inducing or accelerating a healing process of a damaged skin or skin wound, comprising a pharmaceutically acceptable carrier and therapeutically effective amounts of the N-myristoylated PKCα pseudosubstrate peptide of SEQ ID NO: 1 and insulin.

89. A pharmaceutical composition for topical application for inducing or accelerating a healing process of a damaged skin or skin wound, comprising a pharmaceutically acceptable carrier and therapeutically effective amounts of the N-myristoylated PKCα pseudosubstrate peptide of SEQ ID NO: 1, insulin and Copolymer-1.

90. The pharmaceutical composition of claim 52, wherein said skin wound is selected from the group consisting of an ulcer, a diabetes related wound, a burn, a sun burn, an aging skin wound, a corneal ulceration wound, an inflammatory gastrointestinal tract disease wound, a bowel inflammatory disease wound, a Crohn's disease wound, an ulcerative colitis, a hemorrhoid, an epidermolysis bulosa wound, a skin blistering wound, a psoriasis wound, an animal skin wound, a proud flesh wound, an animal diabetic wound, a retinopathy wound, an oral wound (mucositis), a vaginal mucositis wound, a gum disease wound, a laceration, a surgical incision wound and a post surgical adhesions wound.

91. The pharmaceutical composition of claim 90, wherein said ulcer is selected from the group consisting of a diabetic ulcer, a pressure ulcer, a venous ulcer, a gastric ulcer and an HIV related ulcer.

92. A method of inducing or accelerating a healing process of a damaged skin or skin wound, the method comprising administering to the damaged skin or skin wound area a therapeutically effective amount of Copolymer-1.

93. The method of claim 80, wherein said administering is effected by a single application.

94. The method of claim 92, wherein said therapeutically effective amount of Copolymer-1 is a concentration of Copolymer-1 ranging between 1 to 500 μg/ml.

95. The method of claim 92, wherein said wound is selected from the group consisting of an ulcer, a diabetes related wound, a burn, a sun burn, an aging skin wound, a corneal ulceration wound, an inflammatory gastrointestinal tract disease wound, a bowel inflammatory disease wound, a Crohn's disease wound, an ulcerative colitis, a hemorrhoid, an epidermolysis bulosa wound, a skin blistering wound, a psoriasis wound, an animal skin wound, a proud flesh wound, an animal diabetic wound, a retinopathy wound, an oral wound (mucositis), a vaginal mucositis wound, a gum disease wound, a laceration, a surgical incision wound and a post surgical adhesions wound.

96. The method of claim 95, wherein said ulcer is selected from the group consisting of a diabetic ulcer, a pressure ulcer, a venous ulcer, a gastric ulcer and an HIV related ulcer.

97. The method of claim 92, wherein said Copolymer-1 is contained in a pharmaceutical composition adapted for topical application.

98. A pharmaceutical composition for topical application for inducing or accelerating a healing process of a damaged skin or skin wound, comprising a therapeutically effective amount of Copolymer-1 and a pharmaceutically acceptable carrier.
